SEQUENCE LISTING (22 sequences)

SEQ ID NO: 1 - protein, 221 aa, Artificial Sequence
  Myomaker - Human
MGTLVAKLLLPTLSSLAFLPTVSIAAKRRFHMEAMVYLFTLFFVALHHACNGPGLSVLCFMRHDILEYFSVYGTALSMWVSLMALADFDEPKRSTFVMFGVLTIAVRIYHDRWGYGVYSGPIGTAILIIAAKWLQKMKEKKGLYPDKSVYTQQIGPGLCFGALALMLRFFFEDWDYTYVHSFYHCALAMSFVLLLPKVNKKAGSPGTPAKLDCSTLCCACV

SEQ ID NO: 2 - protein, 221 aa, Artificial Sequence
  Myomaker - Dog
MGTLAAKLLLPTLSSLAFLPTVSIAAKRRFHMEAMVYLFTMFFVALHHACNGPGLSVLCFMRHDVLEYFSVYGTALSMWVSLMALADFDEPKRSTFVMFGVLTIAVRIYHDRWGYGVYSGPIGTAVLIIATKWLQQMKEKKSLYPDKSVYTQQIGPGLCFGALALMLRFFFEDWDYTYVHSFYHCALAMSFVLLLPKVNKKAGSAGPPAKLDCSTLCCACI

SEQ ID NO: 3 - protein, 221 aa, Artificial Sequence
  Myomaker - Pig
MGTVMAKLLLPTLSSLAFLPTVSIAAKRRFHMEAMVYLFTTFFVAFYHACHGPGLAMICFLRLDILEYFSVYGTALSMWVSLMALADFDEPKRSTFVMFGVLTIAVRIYHDRWGYGVYSGPIGTAALIIAAKWLQQMKDQRRLYPDKSVYTQQIGPGLCFGALALMLRFFFEEWDYTYVHSFYHCALAMSFVLLLPKANKKAGSAGPPAKLDCSTLCCACI

SEQ ID NO: 4 - protein, 221 aa, Artificial Sequence
  Myomaker - Mouse
MGTVVAKLLLPTLSSLAFLPTVSIATKRRFYMEAMVYLFTMFFVAFSHACDGPGLSVLCFMRRDILEYFSIYGTALSMWVSLMALADFDEPQRSTFTMLGVLTIAVRTFHDRWGYGVYSGPIGTATLIIAVKWLKKMKEKKGLYPDKSIYTQQIGPGLCFGALALMLRFFFEEWDYTYVHSFYHCALAMSFVLLLPKVNKKAGNAGAPAKLTFSTLCCTCV

SEQ ID NO: 5 - protein, 221 aa, Artificial Sequence
  Myomaker - Opossum
MGTLVTKLLLPTISSLAFLPTISIAAKRRFHMEAMVYLFTMFFIAIYHACDGPGLSVLCFMRYDILEYFSIYGTALSMWVSLMALAEFDEPKRSTFVMFGVLTIAVRIYQDRWGYGVYSGPIGTAVLIIATKWLQKMKEKKGLYPDKSVYTQQIGPGFCFGALALMLRFFFQEWDYTYVHSFYHCSLAMSFVLLLPKVNKKAGNAGTPAKLDCSTLCCACI

SEQ ID NO: 6 - protein, 220 aa, Artificial Sequence
  Myomaker - Zebrafish
MGAFIAKMLLPTISSLVFVPAASVAAKRGFHMEAMVYFFTMFFTAIYHACDGPGLSILCFMKYDILEYFSVYGTAISMWVTLLALGDFDEPKRSSLTMFGVLTAAVRIYQDRLGYGIYSGPIGTAVFMITVKWLQKMKEKKGLYPDKSVYTQQVGPGCCFGALALMLRFYFEEWDYAYVHSFYHVSLAMSFILLLPKKNRYAGTGRNAAKLNCYTLCCCV

SEQ ID NO: 7 - DNA, 666 bp, Artificial Sequence
  Myomaker - Human cDNA
atggggacgctggtggccaagctgctcctgcccaccctcagcagcctggccttcctccccactgtcagcatcgcggccaagaggcggttccacatggaggccatggtctacctcttcaccctgttcttcgtggcgctccaccatgcctgcaatggacccggcttgtctgtgctgtgcttcatgcgtcacgacatcctggagtatttcagtgtctacgggacagccctgagcatgtgggtctcgctgatggcactggccgacttcgacgaacccaagaggtcaacatttgtgatgttcggcgtcctgaccattgctgtgcggatctaccatgaccgatggggctacggggtgtactcgggccccatcggcacagccatcctcatcatcgcggcaaagtggctacagaagatgaaggagaagaagggcctgtacccagacaagagcgtctacacccagcagataggccccggcctctgcttcggggcgctggccctgatgctacgcttcttctttgaggactgggactacacttatgtccacagcttctaccactgtgccctggctatgtcctttgttctgctgctgcccaaggtcaacaagaaggctggatccccggggaccccggccaagctggactgctccaccctgtgctgtgcttgtgtctga

SEQ ID NO: 8 - DNA, 666 bp, Artificial Sequence
  Myomaker - Dog cDNA
atggggacgctcgcggcgaagctgctcctgcccaccctcagcagcctggccttcctccccaccgtcagcatcgccgccaagcggcggttccacatggaggccatggtctacctcttcaccatgttcttcgtggcactccaccacgcgtgcaacgggcccgggctatcggtgctctgcttcatgcgccacgacgtcctggagtacttcagcgtctatgggacggcactgagcatgtgggtctcgctgatggcactggctgacttcgacgaacccaagaggtcgacttttgtgatgtttggcgtcctgaccatcgccgtgcggatctaccatgaccgctggggctacggggtgtactcgggccccattggcacggctgtcctcatcatcgccacaaagtggctgcagcagatgaaggagaagaagagtctgtacccggacaagagtgtctacacccagcagataggccctggcctctgttttggggcactggcccttatgctgcgcttcttttttgaggactgggattacacctatgtccacagcttctaccactgtgccctggccatgtccttcgtcctcctgctccccaaggtcaacaagaaggctggaagcgcggggccccctgccaagctagactgctctaccctttgctgtgcttgcatctga

SEQ ID NO: 9 - DNA, 666 bp, Artificial Sequence
  Myomaker - Pig cDNA
atggggaccgtcatggccaaactgctgctacccacgctgagcagcctggccttcctccccacggtcagcatcgctgccaagcggcggttccacatggaggccatggtctatctcttcaccacgttcttcgtggcgttctaccacgcctgccacgggccgggcctggctatgatctgctttctgcgccttgacatcctggagtatttcagcgtctacggaaccgccctgagcatgtgggtctcgctgatggcgctggctgacttcgacgagcccaagaggtcgactttcgtgatgtttggcgtcctgaccatcgccgtgcggatctaccacgaccgctggggctacggcgtgtactcgggccccatcggcacggccgccctcatcatcgcggccaagtggctgcagcagatgaaggaccaacggcgcctgtatccagacaagagcgtgtacacacagcagataggccccggcctctgcttcggggcgctggccctcatgctgcgctttttcttcgaggagtgggattatacctacgtccacagcttctaccactgcgccctggccatgtccttcgtcctgctgctgcccaaggccaacaagaaggctggaagcgcagggccacccgccaagctggactgctccaccctctgctgtgcttgtatctga

SEQ ID NO: 10 - DNA, 666 bp, Artificial Sequence
  Myomaker - Mouse cDNA
atggggacagttgtagccaaactgctcctgcctaccctcagcagcctggccttcctcccgacagtgagcatcgctaccaagaggcgtttctacatggaggccatggtctacctcttcaccatgttctttgtggcgttctcccatgcctgtgatgggcctggtttgtctgtgctgtgcttcatgcgccgtgacattctggagtacttcagcatctatggaacagccctgagcatgtgggtctccctgatggcactggccgactttgatgaaccccagagatcgaccttcacaatgcttggcgtccttaccatcgctgtgcggacttttcatgaccgctggggttacggggtatactccggtcccataggcacggccaccctcatcattgctgtaaagtggctgaagaagatgaaagagaagaagggcctgtaccccgacaagagcatctacacccagcagataggccccggcctgtgctttggggccctggccctgatgcttcgattcttctttgaggaatgggattacacctacgtccacagcttctaccactgtgccctggccatgtcctttgtcctgctgctgcccaaggtcaacaagaaggctgggaacgcaggggcccccgccaagctgaccttctccaccctctgctgcacttgtgtctga

SEQ ID NO: 11 - DNA, 666 bp, Artificial Sequence
  Myomaker - Opossum cDNA
atggggactcttgttaccaagttgcttcttcccacaatcagcagcctcgcctttctccccaccatcagcatcgctgctaagaggagattccacatggaagccatggtctacctcttcaccatgttcttcatagcaatatatcatgcatgtgacgggccaggcttatcagtgctatgcttcatgcgctatgacatactggagtatttcagcatctatgggacagcactgagcatgtgggtgtcattaatggcactggcagagttcgatgaaccaaaaaggtcaacctttgtaatgtttggcgtgttgactattgccgtgaggatctaccaagaccggtggggatatggggtatactcggggcctattggcacagctgtccttatcattgcaacaaaatggctgcaaaagatgaaagagaagaagggtctgtaccctgacaagagtgtgtacacccaacagataggccctggtttctgttttggagcgttagcactgatgctgcgtttctttttccaggagtgggattacacctatgttcacagcttctaccactgttcactagccatgtcctttgtcttgctgctgcccaaggtaaacaagaaagctgggaatgctgggacacctgccaaattggactgttctacactctgctgtgcttgcatctga

SEQ ID NO: 12 - DNA, 663 bp, Artificial Sequence
  Myomaker - Zebrafish cDNA
atgggagcgtttatcgccaagatgttgctgcccactattagcagtttggtgtttgtgcctgcagccagcgtggctgcaaagaggggcttccacatggaggccatggtctatttcttcacaatgttcttcaccgcgatttaccacgcatgtgacggtccgggcttgtccattctctgtttcatgaagtatgacattctggagtacttcagcgtgtacgggacagccatctccatgtgggtcacgctactggcgcttggggatttcgatgagcccaaacgctcttcgctcaccatgtttggggtgttgaccgcagctgtgaggatttaccaggaccgactgggctacggcatttactccggccccatcgggacagctgtctttatgatcacagtcaaatggttacagaaaatgaaggaaaagaaaggcctttatccagacaaaagtgtttacactcaacaagtgggcccagggtgctgcttcggtgctcttgctttgatgcttcgcttctattttgaggagtgggactacgcttatgttcacagtttctaccacgtgtctctggccatgtcctttattctgctgctgcccaagaagaaccgttatgctggaacgggacgtaacgcagccaaactcaactgctacaccctctgctgctgtgtatga

SEQ ID NO: 13 - DNA, 10361 bp, Artificial Sequence
  Myomaker - Human (+strand)
caagtgtgagctggggagggcaggggctcagagccgggctgggcgcagcatcagacacaagcacagcacagggtggagcagtccagcttggccggggtccccggggatccagccttcttgttgaccttgggcagcagcagaacaaaggacatagccagggcacagtggtagaagctgtggacataagtgtagtcccagtcctgcggggggcaagcggtcagtctggggcctcagccccctccccgaggctcctccctctccaagacccagcagagccccttcaggcccccgcctctgccagggcactgggacacctgcaggaagcctcccccacggtcgcgctcacagtggtttttctctccacctaaacccagagcagtgagggcctgtgccatcctccaggctgcactccttccttcttccccatcccctctctctgctgtccttctcttcctccatccttctctccctcctaccctccctccctccatctccccctcttttctctccttatccctcttcccctgttcctccctccctcctccactttctccctccttccttccctgtctcctcccctccctccctccctcctccaggtgttgggcacctgccccaggcgtctcccaggctgtgctgccgtctgagatgccagctgtctgtaggcagccagctttggtctctgtgacctccaggtccacacaggccatggtgctggtggtgctggggacggcattgcccccgacatagccctgggaggggctagtgagcagggactaataccagactttggcctggggctgtcagagtccccccagcgtgggcacagccctggtatcccagctgagcagagccatgccgagtgggctctggggcacaggacacctccccgctgggcttggtacctcaaagaagaagcgtagcatcagggccagcgccccgaagcagaggccggggcctatctgctgggtgtagacgctcttgtctgggtacaggcccttcttctccttcatcttctgtagctgtgaggacaggaggccacagcaaagcttttaggtcacagcactggggaacgcccctccccaaaccagcccgagagctggccctgcacaggctcaccccagccctctcccggcaggagaggaggctcaggagcctcctgccgcacccagcctcagatggcttctgctggacagggcccttcacggtgcgacccagcagagaccccagcctggatggctgggaaggaagccactgggccatgtgccccacaaagaccccgctgccctcccgcctctttgagatgtaacaacgccaccctcgcatgtctcctcctccctggaggggagctctggggggactagactccatgattgcttaccaaggaaagtactggagtacttgggacctgccagcccagtgtggcccatggggatggcacttgtggtgatccctgagccatggacaagcatcgtttgctttcctagttaaaggacctatctcactcttcattagacaaacttggccagcactgcttctcaggtcccagtgcttaggaaggctcgcgtgggcgtttccacttacagaggggtttgcattccgaggaagatgcgggaagtgtggggccacatccctggagccggccttgtgttttctaggccacttcacatggagtctatttgggattttcaagggcagttgtttcctggaatgagggtggatttttctccctgagcctggtcccctcttgggaggggctggggaacgacagccttgttggggaggaaggagggagggttgggtgatggcggcctcggagtggggccagacccgtgggggtacactcaggaggctatagatttcagtggaatcaactgttagacacacagcgtgtggcacaagcccctgggggtgggggcagcaccccataactgcacccattgctgagtggcctatgcaaagagcacaaagagccttatgctgggtcaggtcaggttttgccacccagtgaattatgaattgatgcccggctttccattttctggaattccattgccaacaaggaattgagcacctgcagtcctgcagtggcctgaagacagctggaccgtgtgaccctgggtgcggtggtcaaggctgccagcccacctctggccagccctgcagtagtaacaccagggagaagagaggtgcctgccccaggtcacacagtgggcctggcactattgaaagggcgccatcacccaaccctccctccttcttcctcccgggctgccattgcccaacccctcccaagaggggacgaggctcagggagaaaaatccaccctcattccaggaaacaattgcccttgaaaatcctaaataaactccatactaaatggtctagaagacaacaatttgagccccagatgcggggaggcgggcagcccatcctcggctcctgtggctggatctgcagcctgagggccttggcagtctcgtggctcttggtgggaaacacagcagtgaattctcttctgggcaattacagttcagcccagttcagacctggccaagaccagcgggaggagcaaccttcaggggcagaaggaggcgagaggcgggtggccaggacccagggccccagcacgctccttcctgccacccaccttggtccagcccacttatgcccagcgctccctctctccccaccaggtgactcccaggggcctcctgggtcagcccaggattagtgctgcttcctcaggttgcagacagaaagcaggtcctctgtctcctgctcaaaaagtcaagtccagccaggcgtggtggctcatgcctgtaattccagcactttgggagactgaggcaggcagattacctgaagtcaggagctcaggaccagccgggccaacgtggtgaaaccccatcgctactaaaaatataaaaattacctgggcgtgatggcatgcgcctataatcccagctactcgggaggctgagacaggagaatcgcttcaacccgggaggcggaggttgcagtgagccaagatggcgccattgcactccagcctgggtgacaagagcaaaactccgtctcaaaaaaaaaaaaaaaaaagtcaggttctggccccgccactgccctgccatgacgtcctgttaagttgctgaggcctccatgctttggttccttcataggccaaatggcaaatcagtcccatgctccttggctgtggggaggattgggacgggctttgcaagctgcccaccagaactcgagcgctctccccacagccgtgggccctcctgcactgagagctgccctctgtcttgctgggtgtcctgcggctctggccggggctggcagtgtggctgggctggaccaggccaggtcctctcttggcacttgaaactgaccctgagacttcaggtccactccaaagaggtgaaatgcagcacagggatgttcaggcggtgcctgggctgctgcaggcctggagagcaggctcaggctgaagcctgctggctccccaggtctgggagacccttgcaagggtgagctccctcctgctctggggtcccaggagatgccccgggtctatttttccctaagatccctctttagcttgggcgagtttgagtggggtttggtccctgagccaggagggtcttggtaggacggagagagcagggagcactgaagaccacgtgagggccttgctgctctgcaaggggctgtctgtgctagaaggtctggcccaggctgcctcactgtcataccacactctccctcctggctagaaccaagctcgaggctcactccctccaggaagtcttcccagattaccccaggccattttccaagttgatgttgcatctctaaagcagctggtagtaagagcggtgatgagagtgataacaaatagctcttatgtgcggagcacattggaagccaggctccatgccaggacttcaggtgcctgatctcagtgagtctttgaaccaccccatgagacaggcagggggctgtaatgacaacacctgctttacaggtacgggcgtggaggtgagacattgggtaacttgggctcagtctggagctggtgagtacagacaagcgtcacacacagtctacacagccggagcacctcatggctattttctacgtggttttgctgaattcctgcatccacccatttgcctatgagggcaggaggtaaatgaagatccgaggcaggaggagtcagacaggggagaggtgacgggcctcctgggtccccgttcatcgaggctcgcgcagtacgcacccactttgccgcgatgatgaggatggctgtgccgatggggcccgagtacaccccgtagccccatcggtcatggtagatccgcacagcaatggtcaggacgccgaacatcacaaatgttgacctcttgggttcgtcgaagtcggccagtgctggaggggccagggagacacagggggaggtgagtggtctctcttgctcctcctggctacccccccaccccccagcccccaggaggcatcctgtagatgccctctctcggtgtcccctcagccagcgagaccctgaggcccagcctggtcatggaggggtctgaattccagccagtttgagaggacaggcagcctgctgcttccccatggacacagcagcttggattgtgctcccagcacctcattttaataaacagaccacagctggttgtggtggctcaggtctgcaatcccagtgctttgggaggcagaggcaggaggatcgtctgagaccaggagttcaagactagcctgggcaacatagcgggacccccatctccacaaaaaattcggtgggtgtcgtggtgcatgcctgtcatcccagctacttgggaggctgaggtgggaggatggcttgaggctgtgagttcgaggctgcagtgagccgtgtttgtgccactgtactctggcctgagtgacagagtgagaccctgtggctaaaaatcaataatcactatgcaaagtgaataggatcgaatctatcccataggatcacaggacaaagacactaagattcaagagaagaaatgaagcccctcacaggcccggttagatggcaaggagcctcaggtcatggggaccttgccacagacaacagttacgtggaaaaaaacatggtgggaaagggggcttatgaacagtcccgtcttccaggctggatatcacccgtgtgtgtggatgtttgtatgacagtctgggaagccaacccccctgagcagtgaacagcggtcctcccagggaaggagtgacgggagggagccctttcactttttcctttgtatgcctctgctgttgaaatgtgtcacaacaagcttttactaaatgagtcattttaaaaggatataaaaaatcggcatcagggcatttaagaggtgcatattctttttcatagattaagcacaaccctgaaacccagacaagggaagacattcctggggctgggagtgagtggggatagagggctgcagcgggactggtttgaggctgggtgtgcggacactggggagccggtccttgtccgcaaggcttgtctgcaggggttgaccactcacccatcagcgagacccacatgctcagggctgtcccgtagacactgaaatactccaggatgtcgtgacgcatgaagcacagcacagacaagccgggtccattgcaggcatggtggagctgccagaaaacccacaggtggtcacagcacaaagaggccagagctggtccccgagccacggcccccagagtgccaggtcacttgctggctgtgagaagtcactttggcgagtcacttaatgactgtgtgcctcagtctccccgtctgaaaaatgggggtactgccgagcactcccgcagagggtcctgtggggattaagtggcacatgccagcgaggtgtttaggggctggggtgtgccaagggttcactcaatgtcacctcagcagaattcgctcatctgcactggcaggactgggcggagactgagtggtcactcaggtgaagcccgcttaggtggggcggtctccgggagggaccctacacggctctccccggaccttcagcatctgtgcttccttgaagcacacagctgcgtgttcactcgccaatctttggatgtgaggtcagagcctctctgggggctcctttgctctttgggggctcctggggccttctcttgcacaaattacccctctgatgactggtctacactgcagcagcgttctcaggcttgagtgggcatcagaacgcctggggccttgtttagacacaggttactgagccctgcctagggttgctgattagggagggctgggttgggtagaaaatgtgcattttgaacacattccctgtggcactgctgaggctggcagggcccacactgagagccgggctgtagctcctggtttctgttgccttaacgtggacgaagatctctgagacccccttgcagaagctgaacacagccccctaggctcatccatctctgccctatactctcgtcgtcgcctccccaacacccactttcatggcaatttttaaggcaaaaggcttatagggagtgttttcaaagcagtcaactacttttctacggaaaacaactctctctccttttgcattcgcatttcatcattttaggtaatatttaattacatgacataattattttgacaggttcaactggcacaaacaagcttgggaaacagcacggtggactcttggtcagcccagctcagcgggaggagcaggcgtgctggaaagcagcccgtgtctggaggcgacagggacagcacagagggagcgggggccctgggtgatctggggggcaggcaattcggggtcaaagtggagtgcttctactgatggcaattgtacacggcctaaagtgacggtgcacctaggaggcattaatagggatccagcatctaaaatgagggaggcggcggtcctgcttctctctgttcttgtcaggctcatccagaagactatgccgagctctgtgtggtgcacctttcttgaagtgagactgggaaagacgcggctagaggagggtgaccagcggtggacatgactgtttaccttggggacagggaagcttcaggaggggcctgatcaaggtgcttacacctctgtgggaaagaggagcgaggaagactccggccctaggctgttctcctgttctcctggcttcttcccatcccccaccccagccccatcacctgctgtctgtgtgcctcaatgtagcacagatggtcatgtgtgattaaggcattcactgtgagattgtgataaggcctgtgcccttgccctgccaggagcaggaatggctctgtctggtcccagttgcatggacggctcccagcatagagtgcttgctgcatgtgttcagggagggggacgccaggctctgagaattctaaaggacagccagctcaccctggggacccagagcctctgccactaggcccttggctcctcccaatggtgggaacttagctccattcgacagatggggaaagtgaacttcagagcagcactgcctgcccaaagaggtgaaacagagcagtgcttggcacctggccacttcctcccatcctgcagtgcagggggcagacctggcccagccggggcactggtggggtgggtgcggctgagggcctggggggtcagagctcaggctcggggagtctgactttgcagatgttcccagtgggggctcaggtgagtggctgtcgggggggggcctcctctgttgtgtggggacaagcacactgtctccgtggggtttgcacccatagcaaggtgtccggcacagagatggagattgtcacgggaggggcctgattggaagggaagggacgccatgcgggtggcagaactttgggagggactgagtgtggctttgagttcagaagacgtttgtcacaagaggcagctgcccctgccactctgggtggggcagggtggggcctctgagaccagtgcagaggcagctgcggggccagcctaggcccaggcagggaggtgtggcctggtgggtgcttgtggtttgctgggctaggtctaacaggagccttgagaacaagacctcagcttttctccctgcgctaaggccatgggacctgcagagaaatcctggctctgctctgggcttcagtctctcatctgcccaagaggcttcctagccctagcccaggctggagtcccagaggagcgaatgcagtggcatttgggtgagtcaggagctctggagagcttgatggtcacagtgacacaagtgactctgtctctctgggatttggtttcttcatctgccaaatgggaatcaagatcctaggcttgtggggaaggtgaaaaggctgaatcagacactgtgcacagagcgcctagccgagtcctctgccctgggtactggcgctcgaggtggactcagaagctccagggcatctggttccacaaaggacccagcctgtcccaggccactgtcacccctgggagtggcacacactggagggaatgcctcgctcccagcccacacgtgcacactcagcttctgccattgcgggcaaaattggacttgaccaattcaggatacaagcataacatgtgaatatatgcttgcaaacacacgtgtgagctcacgggcctcacccgctcaggactccctctgtgcactcacatgcacttggcattcttgcccatagaggccctgctgctggagaaggaggctgtctggggagaggaggtggagttttcacaggttgggcccagcactgccccaagaaggaggctagtgggacgcttgcctccccagagcaggtgtcatgctggggattgggctgtcagtgaaggaggggtgtgatggaaggtgagcaaggaaggcttcgggagagcaagaggtggggcaccacttgtgggagtccaggagtgagggcatgttagtggagaaagtcggaaagacccagaggcaagaaggcagggggtaccgagacatataaatgatggctgaatggcgagatggtaatagacgaatagatcacaggtagatggatgcgtagatagagagatagatggagagagagagagagagagagagagagagagagagagacaagctggagaaggtggatagctaaagccagagagacacatggagagtcaggggactaaaaccagggagggtgggaccaagagctttagagagagtgaattccatggggatcgagttccagaaatcaaaagagaaccagacagagagagaaaggaaaaaaagagaaacagagaaaactagacacagaaaaccaatacgagaaacacagagtgaaagagacccagaaagagagagagagaagacaggggagacaggggtcccagaaacagcgacctcagaaacaaggacagatggggttctgggcgctccactgaaagccggataagatcacccaatgacaggtaccaggaaacagagagcaggagagagccagagagagagcaagcggagacagtcagccagccagacacataaatagaaagagaaagacggacccacagagagagaagtaggccccagaagagggagagaccagcaggccctcctgaaccagagcagctccaggattctggaatcagactcactcacccaggccttcacactccctgaaccctgcagaccccttcccaggcctggcttgccccactcatctctgctccatcgtggcctatgggtagagctcgaagagaggtggggaggggaggtggccccatgggcagccgtgggggctttgattagcagctgagaaaaggggcacgctggaagggtttatcctcaactcaatggccctgcttcaccccaggcttggtctcacacaggcagtgatcccagagcaacttcctggcacagatgggaaaactgaggtccagataggggaagggactcccctagtcctctctcttcagtctccagaccccacctgggcctgctgtttcattttcaaatcacttctgctcatcacccaatacaagaacgctgtggacagagagcctctcctctacctccaggatggggcctgtgtgggacttcctcccagcccccagactcaccgccacgaagaacagggtgaagaggtagaccatggcctccatgtggaaccgcctcttggccgcgatgctgacagtggggaggaaggccaggctgctgagggtgggcaggagcagcttggccaccagcgtccccatgggccaggaggaaagcactggctggggtggggagggtgctggtgtcccaggtccccagcacaggagcacgaagtgggaaggccagctccctttgggcagggc

SEQ ID NO: 14 - DNA, 10361 bp, Artificial Sequence
  Myomaker  - Human (-strand, reverse complement)
gccctgcccaaagggagctggccttcccacttcgtgctcctgtgctggggacctgggacaccagcaccctccccaccccagccagtgctttcctcctggcccatggggacgctggtggccaagctgctcctgcccaccctcagcagcctggccttcctccccactgtcagcatcgcggccaagaggcggttccacatggaggccatggtctacctcttcaccctgttcttcgtggcggtgagtctgggggctgggaggaagtcccacacaggccccatcctggaggtagaggagaggctctctgtccacagcgttcttgtattgggtgatgagcagaagtgatttgaaaatgaaacagcaggcccaggtggggtctggagactgaagagagaggactaggggagtcccttcccctatctggacctcagttttcccatctgtgccaggaagttgctctgggatcactgcctgtgtgagaccaagcctggggtgaagcagggccattgagttgaggataaacccttccagcgtgccccttttctcagctgctaatcaaagcccccacggctgcccatggggccacctcccctccccacctctcttcgagctctacccataggccacgatggagcagagatgagtggggcaagccaggcctgggaaggggtctgcagggttcagggagtgtgaaggcctgggtgagtgagtctgattccagaatcctggagctgctctggttcaggagggcctgctggtctctccctcttctggggcctacttctctctctgtgggtccgtctttctctttctatttatgtgtctggctggctgactgtctccgcttgctctctctctggctctctcctgctctctgtttcctggtacctgtcattgggtgatcttatccggctttcagtggagcgcccagaaccccatctgtccttgtttctgaggtcgctgtttctgggacccctgtctcccctgtcttctctctctctctttctgggtctctttcactctgtgtttctcgtattggttttctgtgtctagttttctctgtttctctttttttcctttctctctctgtctggttctcttttgatttctggaactcgatccccatggaattcactctctctaaagctcttggtcccaccctccctggttttagtcccctgactctccatgtgtctctctggctttagctatccaccttctccagcttgtctctctctctctctctctctctctctctctctctctccatctatctctctatctacgcatccatctacctgtgatctattcgtctattaccatctcgccattcagccatcatttatatgtctcggtaccccctgccttcttgcctctgggtctttccgactttctccactaacatgccctcactcctggactcccacaagtggtgccccacctcttgctctcccgaagccttccttgctcaccttccatcacacccctccttcactgacagcccaatccccagcatgacacctgctctggggaggcaagcgtcccactagcctccttcttggggcagtgctgggcccaacctgtgaaaactccacctcctctccccagacagcctccttctccagcagcagggcctctatgggcaagaatgccaagtgcatgtgagtgcacagagggagtcctgagcgggtgaggcccgtgagctcacacgtgtgtttgcaagcatatattcacatgttatgcttgtatcctgaattggtcaagtccaattttgcccgcaatggcagaagctgagtgtgcacgtgtgggctgggagcgaggcattccctccagtgtgtgccactcccaggggtgacagtggcctgggacaggctgggtcctttgtggaaccagatgccctggagcttctgagtccacctcgagcgccagtacccagggcagaggactcggctaggcgctctgtgcacagtgtctgattcagccttttcaccttccccacaagcctaggatcttgattcccatttggcagatgaagaaaccaaatcccagagagacagagtcacttgtgtcactgtgaccatcaagctctccagagctcctgactcacccaaatgccactgcattcgctcctctgggactccagcctgggctagggctaggaagcctcttgggcagatgagagactgaagcccagagcagagccaggatttctctgcaggtcccatggccttagcgcagggagaaaagctgaggtcttgttctcaaggctcctgttagacctagcccagcaaaccacaagcacccaccaggccacacctccctgcctgggcctaggctggccccgcagctgcctctgcactggtctcagaggccccaccctgccccacccagagtggcaggggcagctgcctcttgtgacaaacgtcttctgaactcaaagccacactcagtccctcccaaagttctgccacccgcatggcgtcccttcccttccaatcaggcccctcccgtgacaatctccatctctgtgccggacaccttgctatgggtgcaaaccccacggagacagtgtgcttgtccccacacaacagaggaggcccccccccgacagccactcacctgagcccccactgggaacatctgcaaagtcagactccccgagcctgagctctgaccccccaggccctcagccgcacccaccccaccagtgccccggctgggccaggtctgccccctgcactgcaggatgggaggaagtggccaggtgccaagcactgctctgtttcacctctttgggcaggcagtgctgctctgaagttcactttccccatctgtcgaatggagctaagttcccaccattgggaggagccaagggcctagtggcagaggctctgggtccccagggtgagctggctgtcctttagaattctcagagcctggcgtccccctccctgaacacatgcagcaagcactctatgctgggagccgtccatgcaactgggaccagacagagccattcctgctcctggcagggcaagggcacaggccttatcacaatctcacagtgaatgccttaatcacacatgaccatctgtgctacattgaggcacacagacagcaggtgatggggctggggtgggggatgggaagaagccaggagaacaggagaacagcctagggccggagtcttcctcgctcctctttcccacagaggtgtaagcaccttgatcaggcccctcctgaagcttccctgtccccaaggtaaacagtcatgtccaccgctggtcaccctcctctagccgcgtctttcccagtctcacttcaagaaaggtgcaccacacagagctcggcatagtcttctggatgagcctgacaagaacagagagaagcaggaccgccgcctccctcattttagatgctggatccctattaatgcctcctaggtgcaccgtcactttaggccgtgtacaattgccatcagtagaagcactccactttgaccccgaattgcctgccccccagatcacccagggcccccgctccctctgtgctgtccctgtcgcctccagacacgggctgctttccagcacgcctgctcctcccgctgagctgggctgaccaagagtccaccgtgctgtttcccaagcttgtttgtgccagttgaacctgtcaaaataattatgtcatgtaattaaatattacctaaaatgatgaaatgcgaatgcaaaaggagagagagttgttttccgtagaaaagtagttgactgctttgaaaacactccctataagccttttgccttaaaaattgccatgaaagtgggtgttggggaggcgacgacgagagtatagggcagagatggatgagcctagggggctgtgttcagcttctgcaagggggtctcagagatcttcgtccacgttaaggcaacagaaaccaggagctacagcccggctctcagtgtgggccctgccagcctcagcagtgccacagggaatgtgttcaaaatgcacattttctacccaacccagccctccctaatcagcaaccctaggcagggctcagtaacctgtgtctaaacaaggccccaggcgttctgatgcccactcaagcctgagaacgctgctgcagtgtagaccagtcatcagaggggtaatttgtgcaagagaaggccccaggagcccccaaagagcaaaggagcccccagagaggctctgacctcacatccaaagattggcgagtgaacacgcagctgtgtgcttcaaggaagcacagatgctgaaggtccggggagagccgtgtagggtccctcccggagaccgccccacctaagcgggcttcacctgagtgaccactcagtctccgcccagtcctgccagtgcagatgagcgaattctgctgaggtgacattgagtgaacccttggcacaccccagcccctaaacacctcgctggcatgtgccacttaatccccacaggaccctctgcgggagtgctcggcagtacccccatttttcagacggggagactgaggcacacagtcattaagtgactcgccaaagtgacttctcacagccagcaagtgacctggcactctgggggccgtggctcggggaccagctctggcctctttgtgctgtgaccacctgtgggttttctggcagctccaccatgcctgcaatggacccggcttgtctgtgctgtgcttcatgcgtcacgacatcctggagtatttcagtgtctacgggacagccctgagcatgtgggtctcgctgatgggtgagtggtcaacccctgcagacaagccttgcggacaaggaccggctccccagtgtccgcacacccagcctcaaaccagtcccgctgcagccctctatccccactcactcccagccccaggaatgtcttcccttgtctgggtttcagggttgtgcttaatctatgaaaaagaatatgcacctcttaaatgccctgatgccgattttttatatccttttaaaatgactcatttagtaaaagcttgttgtgacacatttcaacagcagaggcatacaaaggaaaaagtgaaagggctccctcccgtcactccttccctgggaggaccgctgttcactgctcaggggggttggcttcccagactgtcatacaaacatccacacacacgggtgatatccagcctggaagacgggactgttcataagccccctttcccaccatgtttttttccacgtaactgttgtctgtggcaaggtccccatgacctgaggctccttgccatctaaccgggcctgtgaggggcttcatttcttctcttgaatcttagtgtctttgtcctgtgatcctatgggatagattcgatcctattcactttgcatagtgattattgatttttagccacagggtctcactctgtcactcaggccagagtacagtggcacaaacacggctcactgcagcctcgaactcacagcctcaagccatcctcccacctcagcctcccaagtagctgggatgacaggcatgcaccacgacacccaccgaattttttgtggagatgggggtcccgctatgttgcccaggctagtcttgaactcctggtctcagacgatcctcctgcctctgcctcccaaagcactgggattgcagacctgagccaccacaaccagctgtggtctgtttattaaaatgaggtgctgggagcacaatccaagctgctgtgtccatggggaagcagcaggctgcctgtcctctcaaactggctggaattcagacccctccatgaccaggctgggcctcagggtctcgctggctgaggggacaccgagagagggcatctacaggatgcctcctgggggctggggggtgggggggtagccaggaggagcaagagagaccactcacctccccctgtgtctccctggcccctccagcactggccgacttcgacgaacccaagaggtcaacatttgtgatgttcggcgtcctgaccattgctgtgcggatctaccatgaccgatggggctacggggtgtactcgggccccatcggcacagccatcctcatcatcgcggcaaagtgggtgcgtactgcgcgagcctcgatgaacggggacccaggaggcccgtcacctctcccctgtctgactcctcctgcctcggatcttcatttacctcctgccctcataggcaaatgggtggatgcaggaattcagcaaaaccacgtagaaaatagccatgaggtgctccggctgtgtagactgtgtgtgacgcttgtctgtactcaccagctccagactgagcccaagttacccaatgtctcacctccacgcccgtacctgtaaagcaggtgttgtcattacagccccctgcctgtctcatggggtggttcaaagactcactgagatcaggcacctgaagtcctggcatggagcctggcttccaatgtgctccgcacataagagctatttgttatcactctcatcaccgctcttactaccagctgctttagagatgcaacatcaacttggaaaatggcctggggtaatctgggaagacttcctggagggagtgagcctcgagcttggttctagccaggagggagagtgtggtatgacagtgaggcagcctgggccagaccttctagcacagacagccccttgcagagcagcaaggccctcacgtggtcttcagtgctccctgctctctccgtcctaccaagaccctcctggctcagggaccaaaccccactcaaactcgcccaagctaaagagggatcttagggaaaaatagacccggggcatctcctgggaccccagagcaggagggagctcacccttgcaagggtctcccagacctggggagccagcaggcttcagcctgagcctgctctccaggcctgcagcagcccaggcaccgcctgaacatccctgtgctgcatttcacctctttggagtggacctgaagtctcagggtcagtttcaagtgccaagagaggacctggcctggtccagcccagccacactgccagccccggccagagccgcaggacacccagcaagacagagggcagctctcagtgcaggagggcccacggctgtggggagagcgctcgagttctggtgggcagcttgcaaagcccgtcccaatcctccccacagccaaggagcatgggactgatttgccatttggcctatgaaggaaccaaagcatggaggcctcagcaacttaacaggacgtcatggcagggcagtggcggggccagaacctgacttttttttttttttttttgagacggagttttgctcttgtcacccaggctggagtgcaatggcgccatcttggctcactgcaacctccgcctcccgggttgaagcgattctcctgtctcagcctcccgagtagctgggattataggcgcatgccatcacgcccaggtaatttttatatttttagtagcgatggggtttcaccacgttggcccggctggtcctgagctcctgacttcaggtaatctgcctgcctcagtctcccaaagtgctggaattacaggcatgagccaccacgcctggctggacttgactttttgagcaggagacagaggacctgctttctgtctgcaacctgaggaagcagcactaatcctgggctgacccaggaggcccctgggagtcacctggtggggagagagggagcgctgggcataagtgggctggaccaaggtgggtggcaggaaggagcgtgctggggccctgggtcctggccacccgcctctcgcctccttctgcccctgaaggttgctcctcccgctggtcttggccaggtctgaactgggctgaactgtaattgcccagaagagaattcactgctgtgtttcccaccaagagccacgagactgccaaggccctcaggctgcagatccagccacaggagccgaggatgggctgcccgcctccccgcatctggggctcaaattgttgtcttctagaccatttagtatggagtttatttaggattttcaagggcaattgtttcctggaatgagggtggatttttctccctgagcctcgtcccctcttgggaggggttgggcaatggcagcccgggaggaagaaggagggagggttgggtgatggcgccctttcaatagtgccaggcccactgtgtgacctggggcaggcacctctcttctccctggtgttactactgcagggctggccagaggtgggctggcagccttgaccaccgcacccagggtcacacggtccagctgtcttcaggccactgcaggactgcaggtgctcaattccttgttggcaatggaattccagaaaatggaaagccgggcatcaattcataattcactgggtggcaaaacctgacctgacccagcataaggctctttgtgctctttgcataggccactcagcaatgggtgcagttatggggtgctgcccccacccccaggggcttgtgccacacgctgtgtgtctaacagttgattccactgaaatctatagcctcctgagtgtacccccacgggtctggccccactccgaggccgccatcacccaaccctccctccttcctccccaacaaggctgtcgttccccagcccctcccaagaggggaccaggctcagggagaaaaatccaccctcattccaggaaacaactgcccttgaaaatcccaaatagactccatgtgaagtggcctagaaaacacaaggccggctccagggatgtggccccacacttcccgcatcttcctcggaatgcaaacccctctgtaagtggaaacgcccacgcgagccttcctaagcactgggacctgagaagcagtgctggccaagtttgtctaatgaagagtgagataggtcctttaactaggaaagcaaacgatgcttgtccatggctcagggatcaccacaagtgccatccccatgggccacactgggctggcaggtcccaagtactccagtactttccttggtaagcaatcatggagtctagtccccccagagctcccctccagggaggaggagacatgcgagggtggcgttgttacatctcaaagaggcgggagggcagcggggtctttgtggggcacatggcccagtggcttccttcccagccatccaggctggggtctctgctgggtcgcaccgtgaagggccctgtccagcagaagccatctgaggctgggtgcggcaggaggctcctgagcctcctctcctgccgggagagggctggggtgagcctgtgcagggccagctctcgggctggtttggggaggggcgttccccagtgctgtgacctaaaagctttgctgtggcctcctgtcctcacagctacagaagatgaaggagaagaagggcctgtacccagacaagagcgtctacacccagcagataggccccggcctctgcttcggggcgctggccctgatgctacgcttcttctttgaggtaccaagcccagcggggaggtgtcctgtgccccagagcccactcggcatggctctgctcagctgggataccagggctgtgcccacgctggggggactctgacagccccaggccaaagtctggtattagtccctgctcactagcccctcccagggctatgtcgggggcaatgccgtccccagcaccaccagcaccatggcctgtgtggacctggaggtcacagagaccaaagctggctgcctacagacagctggcatctcagacggcagcacagcctgggagacgcctggggcaggtgcccaacacctggaggagggagggagggaggggaggagacagggaaggaaggagggagaaagtggaggagggagggaggaacaggggaagagggataaggagagaaaagagggggagatggagggagggagggtaggagggagagaaggatggaggaagagaaggacagcagagagaggggatggggaagaaggaaggagtgcagcctggaggatggcacaggccctcactgctctgggtttaggtggagagaaaaaccactgtgagcgcgaccgtgggggaggcttcctgcaggtgtcccagtgccctggcagaggcgggggcctgaaggggctctgctgggtcttggagagggaggagcctcggggagggggctgaggccccagactgaccgcttgccccccgcaggactgggactacacttatgtccacagcttctaccactgtgccctggctatgtcctttgttctgctgctgcccaaggtcaacaagaaggctggatccccggggaccccggccaagctggactgctccaccctgtgctgtgcttgtgtctgatgctgcgcccagcccggctctgagcccctgccctccccagctcacacttg

SEQ ID NO: 15 - DNA, 10526 bp, Artificial Sequence
  Myomaker - Mouse (+strand)
ttccaggaactagaatgtatgttaggcgaagctaatgactagtggctgatcaagagtttactgtgaatggcttgatcgaaaacctgcagaagggatgggactcaggcaggggtatgcaaggttcgctggctccagcttcctaagtggagagctttcagagcctgggcaggggttaaaagggcaatcccagtttcctagggaaagcagacgattctgacaggcaggacctgggaaatagataaccctgcatgctgctgggtatttactggtctagggttctctgccaggcacacctatggttgtgaggccttgggggataaagttcttttttttcctgaacagagtgaagcaactggtgaacacagaaccagtgggtccctaagcagcactcagcagaatgcagcaggcctgctggtctcttggggtgtagagaagaccatttctcatgtacaggccgcataacaaagtataggaagtaccttgggagagacagcaggactgccaggcaggaaggcaggggcctggtgtgtgtgtgtgtgtgggggggtatagtcagacacaagtgcagcagagggtggagaaggtcagcttggcgggggcccctgcgttcccagccttcttgttgaccttgggcagcagcaggacaaaggacatggccagggcacagtggtagaagctgtggacgtaggtgtaatcccattcctgtggaggagaatgagtcagtctgggcctccatcccttccctaaaccaagtcctagccatttggtgcctctgtcagccagcccaccctgagaaggtggcagaaaggcttgctgccttcctctgttccatgcctcctgggtgctgggcaccagctcctggttccttccaggacatgcgtgcatcttgggtgcaggcttcctaaagtcagggcctgacttgtccactcaggcagtgaggctagtacactggggatggtgagtaccatcctcaagaggacagaatttacaacttggagcctccatatgtggctgttagttaactatttccagaggctcttgctccccttccccataggccaggtacctcaaagaagaatcgaagcatcagggccagggccccaaagcacaggccggggcctatctgctgggtgtagatgctcttgtcggggtacaggcccttcttctctttcatcttcttcagctgcaggcacaaggtggggacatcaaagttcttggggtgcagcacaggaagggacccctccatgaactgtagaagagccctacccccattcctctgtatgcctgactgatgggactctctgggccaatttcccctgggtcctctactgcccgcatctggtgggctttggcacttcagtggcagacgtgatcagttttcccagctaaggggttttcctctgttaaccttggtttcataggccctgtgtgttcaagcttggtaagatggagtgttacatggaatagatgggagtcccatggttcctcactggaatgcacatccttggggcccaaaggtattttaggtattcaagattgttcaggtttcagtggggaagatcattataaataccactgtcaggtgtgcacagagggcacaggacagcagccctgactgagtgatgtgcacagtgggcacaggacagcggccctaattgcacacctcactaaatacattatatgtacaaatgctgtcaatggcctcgtgcaaatcagggcaagctttgtcactctgagtgatgatatgttgctgtttccaagtgttctaaaacttgccattagtaacaggagtggaggtcccagtgagcagtgccagtgacatgggcaccgcctattagcctgagtgtaggccgtatgaccatcaatcacacagttctaacactggggccccagagaggagaagaatattgaagatcacccatgggccctgtcttgccccgggaacccctatttcccatttcactcagcttcttctccccaaatgttgtattcatgttcctttcctgaaagggtgagacatgggaaagaattgtactccgttctaagaagtaagtccaaaccacctgcctatctaagatctaggagatggggtctgtgccccaggcatgggtggctgcagcccctcactcccattctcaccagagacctggggaggctggcatttagtggaggggggcactggcacatgtatgctatcctggctaattaaaatcccatcaggatgggtgtgctgggcttggacaccagcattcaagaggcagaggcgggcagatctctatgagtttgaagccatccagagatacaaagtgagagtctatctttaaaaacaaacaaacaaacaaacaaacaaacaaacaatcaagtcagatccagaaccagtgaagagcagcaaggggccatgataggcaagacaaagaggcagttatcagagcaagccttcttgtttatgcattccagcttgttaactagccatgcagaagcccaacacctctgccttgggtcagagagggccagcttcggctcctcaaactggagtgggatggaagcttctcccctcgaaagtcaagcacagctgccattacctactagggctgcaggttaggctgctgagctctgtgcatttcaggttcatccttaacttaaaatcagaataagcccgggttcctcggagcccacaggagtaggatgtggcttggaagcttcctccctgactatacctgtccccactttgctgaagatggatcagagctctcccacccctggccctgccactcccctctgacacagacacagacacagacacagacacagacacagacacagacacagacacagacacagacacagacacagacacagacacagacagacacagacacagacagacacagacacagacacagacacagacacagacacagacacagacacagacacagacacagacacagacacagacacacaggcatagacacagacacagacagacacagacacagacacagacacagacacagacacagacacagacacagacacagacacagacacagacacagacacagacgacacagacacagacagacacagacacagacagacacagacacagacagacacagacacagacacagacacagacgacacagacacagacagacacagacacagacacagacacagacgacacagacacagacagacacagacacagacacagacacagacacagacacagacacagacacagacacagacacagatgacacagacacagacgacacagacacagtcacagacacagacacagacgacacagacacagacagacacagacacagacagacacagacacagacgacacagacacagacacagacacagacgacacagacacagacagacacagacacagacacagacagacacagacacagacagacacagacacagacacagacacagacacagacacagacgacacagacacagacagacacagacacagacacagacacagacatagacacagacacagagacacagacacagacaacacagacacagacacagacacagacacagacacagactcagacacagacacagacacagacacagacacagacacagactcagactcagactcagactcagactcagactcagactcagactcagactcagacacacagtcacacagacacacacagacacacacagacacacacacaaaggcacacacacacacaaaggcacacacacacacacacaccccaccgcctgccccaatctgcactgctgtagctctacttccaggaacctgcaagatcccaaatggtgcttcctgcatgaggtagcagacaggtgagaacttgaagcctgagtgctgtctgcttggtctgaagcctgctggcctggaaggtctgtgttttgggcctaactgctctgggtggagctcagaaaacatccctgggtctttcctgctattgggaaatttgttcacgatggctaacttaggtgggttttaggccatggaggtgagagggccttgagaccatagaggggttaggagcctatacagcagagtagatgccaagcgccaggccctcctctaggcctcccactcaataccctgcttcacccccacctcacaccttccttcctcatgagaaccatttccaaggcttgcttctttcgggaagactatccagattaacctatctgctctccaaactggtattatacctgtaagcagtgttgtctctcagaatgataatgatagtgatcttatgttgatgaaaagactgacagtgacagtcatgatgacaaaaggtctcctcagctctgggtatattaaaaatcacacctgtgcctgtgcctgtgcttagaagcattctttatgggtatttggatgcagagcagaggtcaagagaaaaggagttttggctttatccaggaccaataaaccagcagggcatgggacccgagcatgagccaccatttttaggaattttagggtttttggcccaattcttactaattcacctgcatataaggatatggggtataggaccctacataggagaaaccaagatcagggaagaaatgcaggttccgtggtctccgacagtggagatactggaagtactcacccactttacagcaatgatgagggtggccgtgcctatgggaccggagtataccccgtaaccccagcggtcatgaaaagtccgcacagcgatggtaaggacgccaagcattgtgaaggtcgatctctggggttcatcaaagtcggccagtgctggggagaggcatagctatggtgagcagcgtccctcacatggctgtgcctccatccttgggaacctattggtatgtcctctcaatctgtagggccagcctggtttccataaggtctgaattttggttatttggagggagtgggtgatgctgcttccctggagcagggtggctgaaataaactggtagactgagtgaccagcatttcctaggaatcctgagacaaaagtgttaagactaatgattggtgcgcagagctgagtctcaggagggaccccgggactgcatccctgggagacaagggtgagcttgcttggtttctcccttttctctttccttcccttccctttctttcccctttctttgctctctccctccttctctccctgtctcccttcctccctccctccctgtctcctttcctttcttcttccttcatcctttctgctttctacttttctctatctcttccttctttttctttcaaaattttgcagttgctggtaatggaatgaagggcctattgattaccaggcgagcgatctgccattgagctgtatataccccaggtccaaggtgaggattttgaatggtctgccttcctaatacacagagctgagctgacccatgagggcaaatgctcctctgagcctggaggacaagctgggaggctaggtccaggatgcctttggcctctcctttgtatgcttctgttttttaaatgtcacaagtgctaactactggagtcacttaaggatggtggaaatgagagtgcaggcatcagagaaatgtgcatgtctctttaagcagattaagctctgcaaagcagcaaggagggaggatctcagagaggggctgggtactggctggggttcaggactggctcccacccattggccaagatggccacttacccatcagggagacccacatgctcagggctgttccatagatgctgaagtactccagaatgtcacggcgcatgaagcacagcacagacaaaccaggcccatcacaggcatgggagaactgtagggaaatcacatgaggtcagcaggcagtgggcagcccaggagtgggtgagaactggtcccaaggctcaggttcactagctgtgagcccctaatggttttgtacctcagcctcctccctcacactatcagagcccttgtggagattaaacaggtgagtccatctagcctgggagtgcaaaagtctttgtaaatatccctttcagactcagcactggcccaaggctggtgagaagcatgctcagaagggcatccttaaagaccacttacacctttgcccatgactgactgaaagtgtacacattcctatgccagtctttgcataggagccttttatcctggacccctgtctctccataaaagaggaagcccttagattccccccaagcaagtgctgattctgacacactggtttctttcccccatatgccagcaggtgtgtccctgactcgtagttgaatagatttgcttctaagcaaaaggttctatatgcaggatttccaagcagacaacttatttcttgcagaaaacaacttgctctccctttgcttcacatttcatcattttaagtaatatttaattacatgacataattattttgacaagtgcaactggcacaaacaagcccagcagccagcacaatgagctcttggtaagcccaacttagcaggagggagcaggcaagctggaaaacagccttgtctggaggcagcaggggcaccaccgagggaggcaggcggagagctggggaccctggatgatggatgtatcagtcaagcacatagggcctacttagaagctcagagacctcctgctggtcacagttgcacatggactctgtcaatcaatagagagcatccaggggaagggaggaggtggtccagcctctgtgttgggtcagccccagccttgagctttgggttctgcaccttttaaaagggagattggtgaagaggaggttaaccaactaggtatgagctcaggaaaagacaagctttgggttgggccagaccaaggtacgcaagtggagaaggaaaggaactcagctctgggaggggactctgctcttctggctccttacagaaccacatgaccccaccccaccccacctgagcccatcatctgtagcatcttgcttccttctcttgtataggcccccatgaatgagtaaaacttacttactgtgagatcctgggaaacactcatgccttcccccacgaggagaagagcttccttaggcttgatctcaacatagaatacttggctacatgtgaaggccagaggagcaggctttctaacaagggatctaactgtcctcaggccctgaggattaattttttggggggtgggtgacctgtgtgacagtgaacttccctggggaacctcctgcccaaggaggcaggggcaaggctgtgatgtgtaccctttctccccagaggcagggagatctggtccagctggtgccaggctaggacacagctgggtgtgacaggagccctaaccctgctgtcagctcagagctggcagaggggcccaggttctctcaggtctctcaggcccaccttgtctaatggcatgagaacacctgttctgtggggcttacaaggggaccctaacgataactgcggagcatggcaccccacactgcaaaaatgaaatgctgtttaaagtttgctttcattaatcaaactttcccccaacctgaaaccaagttaatatgtgcgttatgggcatttaaacaatgtgcttgccctgggcagaattagctcacctctgggaaaaacaattcaatcgatcttattatgctttgcatttctggtggaggactctagtgagtctttgtgactctttcatgcccgactcagaacagtatatgtttgtgtgagatgtggtgaccaggtctaagaccacgtgtgttagaaacagcaaggtatggagaccatgttgaaagcaaaatgtgggtgtaggctgataatatctgattgtggatttgtgtgctactgagtcaaagggccagagagacagctgtctgctataaaagcctaagactcagatcccattctttttgtccctgtttgttgtgctgttcagcaagtagaaaggatgatattgtctaagattcttagattagaacctgattttagattagatgactatcaggttagaacaggagagggcagaattctttggaatacatcagatccacccgctgtgtaactgacaccaagagtcattcttctattcagcagcagcataccatacaactggtagttgtcatggagagtcctacagcagccacgtggaaggcagaactctgtgaggaacagattgtggctttgaggccagaggacatttgtcataagagacagctggccctgccactctgggtggggtgtggcagggtgggcctccaaggccagtgcagaggcagctgtaggccaattagacccaggcaggcaggggtgacctgattggggctgtgatttgctggactgtatctaacacaggccttgggaacaagaccctggcttatgtccttgaccgtggggtctcatcttggctctgaccttggccaggtctcaagaggaacaaatgacagtgtgggacaaagtactgtggggcagaccaggatctgagtgttcatggtgacactggtggcccagtttctctgagactcagtttcctcttctatcaaattgaaatcactatgttaggctcgtgggtgataatgagtccaaccccaccatggttgctttcttgtgacttatcattggcctaatgtcctcccctactgaagtgaactcaagagccatagagtttccagttccttgggttacctatgggaccaccacaaccaggaggtagacaggtgccaagccctcccccactgttctcagcccacatgcattgtggcttctcccaccactagaaagtcatgccagctgactcaggatatggaacacgcatgtgagcacagatgtgtgagtttgtgggctcactcattgagagccagctggataccttcacatactctatgcccttgccttactgagacctgctgcaggaagggcaggcctaaggagaggatgctagtctctaaaagtttggctctgctctaaggaggagactagcaggctgcttgccaaccctgagcatgtatcctaccagtgtgtgggcctcacaccagacaaactagtgaggcatagtgtgatgagagagaaacgaaggttacagagtggtaaaagagacagtgtgactcctggttagaggatagctgagagggccatcatgagaggtactcagaaggactaaagggcaaagtgagaggaggcctttaagacagagagtagatgggtagatgaatggacagggagagagatggttggttagcagataatagagaaatgatagacagatagacagacagacagatgatggatagacacatagaacaagacaaatgataaatgaatagatgatagacaaaggagatagagagacagaagcaagttgaatgggcaggaagataaagtcaggaagacacagagctctggtcaagaacccaggggagagcagaccagggagaagaggagagtgaactcctcgggggagtgtaactctagaaatcagaaaaaaacaaaaaaaaaaaaccccaaaaaacaaacaaacaaacaaacaaaaaaattggatacaggcaggaagaaggaagagatagagactgggagaaactagacacagaaccagtcaaagaagcagagggagagagacccatggcgggaataaagagaagcagaaacccagacacaaggcttcagcaaagctgggccagtgccagacatgccccgaacgaacgacagaggagtcacccagtactgttgcctgggaacagagtggagaaggaactaagaggcagccagccagctagacacataacaggaagagaaagaaggactcagggagaggctggctcctctcagtgggggtagttccaaattctggagctgcagtcacccaggccctctacctttcctgaacctagtagatccattcctaggcctgctcactcaccttgttcctcctcagctgagcaactcatggaacaacgttggtagaaaggagagagagtctgaggagcaccaggcttgaccttaactgacaccgggctctcatgggcctggcctcagtctcaggtgtcaatcacccccctcaaatgtctggcgcacatggagaaactgaggtccacagaggaagacagattccaggaaccttctcttcccagtcaccacccccactgctcccccagacccagactctttctcttccaaatcctgtttctgcatcacctggcacaggacaatggtggtaaccctcccgtgaggacttcctcctaatttcctccttccacacttaccgccacaaagaacatggtgaagaggtagaccatggcctccatgtagaaacgcctcttggtagcgatgctcactgtcgggaggaaggccaggctgctgagggtaggcaggagcagtttggctacaactgtccccatggaccaggaggaaggcactgactggggagaaggtggtaaaggcccccctggtctccagggcaggaagaaaaagagcccacttctttgcttctccagcagccctgaccgcagctgtggcagcacccacaaggagggcttaagtgctc

SEQ ID NO: 16 - DNA, 10526 bp, Artificial Sequence
  Myomaker - Mouse (- strand, reverse complement)
gagcacttaagccctccttgtgggtgctgccacagctgcggtcagggctgctggagaagcaaagaagtgggctctttttcttcctgccctggagaccaggggggcctttaccaccttctccccagtcagtgccttcctcctggtccatggggacagttgtagccaaactgctcctgcctaccctcagcagcctggccttcctcccgacagtgagcatcgctaccaagaggcgtttctacatggaggccatggtctacctcttcaccatgttctttgtggcggtaagtgtggaaggaggaaattaggaggaagtcctcacgggagggttaccaccattgtcctgtgccaggtgatgcagaaacaggatttggaagagaaagagtctgggtctgggggagcagtgggggtggtgactgggaagagaaggttcctggaatctgtcttcctctgtggacctcagtttctccatgtgcgccagacatttgaggggggtgattgacacctgagactgaggccaggcccatgagagcccggtgtcagttaaggtcaagcctggtgctcctcagactctctctcctttctaccaacgttgttccatgagttgctcagctgaggaggaacaaggtgagtgagcaggcctaggaatggatctactaggttcaggaaaggtagagggcctgggtgactgcagctccagaatttggaactacccccactgagaggagccagcctctccctgagtccttctttctcttcctgttatgtgtctagctggctggctgcctcttagttccttctccactctgttcccaggcaacagtactgggtgactcctctgtcgttcgttcggggcatgtctggcactggcccagctttgctgaagccttgtgtctgggtttctgcttctctttattcccgccatgggtctctctccctctgcttctttgactggttctgtgtctagtttctcccagtctctatctcttccttcttcctgcctgtatccaatttttttgtttgtttgtttgtttgttttttggggttttttttttttgtttttttctgatttctagagttacactcccccgaggagttcactctcctcttctccctggtctgctctcccctgggttcttgaccagagctctgtgtcttcctgactttatcttcctgcccattcaacttgcttctgtctctctatctcctttgtctatcatctattcatttatcatttgtcttgttctatgtgtctatccatcatctgtctgtctgtctatctgtctatcatttctctattatctgctaaccaaccatctctctccctgtccattcatctacccatctactctctgtcttaaaggcctcctctcactttgccctttagtccttctgagtacctctcatgatggccctctcagctatcctctaaccaggagtcacactgtctcttttaccactctgtaaccttcgtttctctctcatcacactatgcctcactagtttgtctggtgtgaggcccacacactggtaggatacatgctcagggttggcaagcagcctgctagtctcctccttagagcagagccaaacttttagagactagcatcctctccttaggcctgcccttcctgcagcaggtctcagtaaggcaagggcatagagtatgtgaaggtatccagctggctctcaatgagtgagcccacaaactcacacatctgtgctcacatgcgtgttccatatcctgagtcagctggcatgactttctagtggtgggagaagccacaatgcatgtgggctgagaacagtgggggagggcttggcacctgtctacctcctggttgtggtggtcccataggtaacccaaggaactggaaactctatggctcttgagttcacttcagtaggggaggacattaggccaatgataagtcacaagaaagcaaccatggtggggttggactcattatcacccacgagcctaacatagtgatttcaatttgatagaagaggaaactgagtctcagagaaactgggccaccagtgtcaccatgaacactcagatcctggtctgccccacagtactttgtcccacactgtcatttgttcctcttgagacctggccaaggtcagagccaagatgagaccccacggtcaaggacataagccagggtcttgttcccaaggcctgtgttagatacagtccagcaaatcacagccccaatcaggtcacccctgcctgcctgggtctaattggcctacagctgcctctgcactggccttggaggcccaccctgccacaccccacccagagtggcagggccagctgtctcttatgacaaatgtcctctggcctcaaagccacaatctgttcctcacagagttctgccttccacgtggctgctgtaggactctccatgacaactaccagttgtatggtatgctgctgctgaatagaagaatgactcttggtgtcagttacacagcgggtggatctgatgtattccaaagaattctgccctctcctgttctaacctgatagtcatctaatctaaaatcaggttctaatctaagaatcttagacaatatcatcctttctacttgctgaacagcacaacaaacagggacaaaaagaatgggatctgagtcttaggcttttatagcagacagctgtctctctggccctttgactcagtagcacacaaatccacaatcagatattatcagcctacacccacattttgctttcaacatggtctccataccttgctgtttctaacacacgtggtcttagacctggtcaccacatctcacacaaacatatactgttctgagtcgggcatgaaagagtcacaaagactcactagagtcctccaccagaaatgcaaagcataataagatcgattgaattgtttttcccagaggtgagctaattctgcccagggcaagcacattgtttaaatgcccataacgcacatattaacttggtttcaggttgggggaaagtttgattaatgaaagcaaactttaaacagcatttcatttttgcagtgtggggtgccatgctccgcagttatcgttagggtccccttgtaagccccacagaacaggtgttctcatgccattagacaaggtgggcctgagagacctgagagaacctgggcccctctgccagctctgagctgacagcagggttagggctcctgtcacacccagctgtgtcctagcctggcaccagctggaccagatctccctgcctctggggagaaagggtacacatcacagccttgcccctgcctccttgggcaggaggttccccagggaagttcactgtcacacaggtcacccaccccccaaaaaattaatcctcagggcctgaggacagttagatcccttgttagaaagcctgctcctctggccttcacatgtagccaagtattctatgttgagatcaagcctaaggaagctcttctcctcgtgggggaaggcatgagtgtttcccaggatctcacagtaagtaagttttactcattcatgggggcctatacaagagaaggaagcaagatgctacagatgatgggctcaggtggggtggggtggggtcatgtggttctgtaaggagccagaagagcagagtcccctcccagagctgagttcctttccttctccacttgcgtaccttggtctggcccaacccaaagcttgtcttttcctgagctcatacctagttggttaacctcctcttcaccaatctcccttttaaaaggtgcagaacccaaagctcaaggctggggctgacccaacacagaggctggaccacctcctcccttcccctggatgctctctattgattgacagagtccatgtgcaactgtgaccagcaggaggtctctgagcttctaagtaggccctatgtgcttgactgatacatccatcatccagggtccccagctctccgcctgcctccctcggtggtgcccctgctgcctccagacaaggctgttttccagcttgcctgctccctcctgctaagttgggcttaccaagagctcattgtgctggctgctgggcttgtttgtgccagttgcacttgtcaaaataattatgtcatgtaattaaatattacttaaaatgatgaaatgtgaagcaaagggagagcaagttgttttctgcaagaaataagttgtctgcttggaaatcctgcatatagaaccttttgcttagaagcaaatctattcaactacgagtcagggacacacctgctggcatatgggggaaagaaaccagtgtgtcagaatcagcacttgcttggggggaatctaagggcttcctcttttatggagagacaggggtccaggataaaaggctcctatgcaaagactggcataggaatgtgtacactttcagtcagtcatgggcaaaggtgtaagtggtctttaaggatgcccttctgagcatgcttctcaccagccttgggccagtgctgagtctgaaagggatatttacaaagacttttgcactcccaggctagatggactcacctgtttaatctccacaagggctctgatagtgtgagggaggaggctgaggtacaaaaccattaggggctcacagctagtgaacctgagccttgggaccagttctcacccactcctgggctgcccactgcctgctgacctcatgtgatttccctacagttctcccatgcctgtgatgggcctggtttgtctgtgctgtgcttcatgcgccgtgacattctggagtacttcagcatctatggaacagccctgagcatgtgggtctccctgatgggtaagtggccatcttggccaatgggtgggagccagtcctgaaccccagccagtacccagcccctctctgagatcctccctccttgctgctttgcagagcttaatctgcttaaagagacatgcacatttctctgatgcctgcactctcatttccaccatccttaagtgactccagtagttagcacttgtgacatttaaaaaacagaagcatacaaaggagaggccaaaggcatcctggacctagcctcccagcttgtcctccaggctcagaggagcatttgccctcatgggtcagctcagctctgtgtattaggaaggcagaccattcaaaatcctcaccttggacctggggtatatacagctcaatggcagatcgctcgcctggtaatcaataggcccttcattccattaccagcaactgcaaaattttgaaagaaaaagaaggaagagatagagaaaagtagaaagcagaaaggatgaaggaagaagaaaggaaaggagacagggagggagggaggaagggagacagggagagaaggagggagagagcaaagaaaggggaaagaaagggaagggaaggaaagagaaaagggagaaaccaagcaagctcacccttgtctcccagggatgcagtcccggggtccctcctgagactcagctctgcgcaccaatcattagtcttaacacttttgtctcaggattcctaggaaatgctggtcactcagtctaccagtttatttcagccaccctgctccagggaagcagcatcacccactccctccaaataaccaaaattcagaccttatggaaaccaggctggccctacagattgagaggacataccaataggttcccaaggatggaggcacagccatgtgagggacgctgctcaccatagctatgcctctccccagcactggccgactttgatgaaccccagagatcgaccttcacaatgcttggcgtccttaccatcgctgtgcggacttttcatgaccgctggggttacggggtatactccggtcccataggcacggccaccctcatcattgctgtaaagtgggtgagtacttccagtatctccactgtcggagaccacggaacctgcatttcttccctgatcttggtttctcctatgtagggtcctataccccatatccttatatgcaggtgaattagtaagaattgggccaaaaaccctaaaattcctaaaaatggtggctcatgctcgggtcccatgccctgctggtttattggtcctggataaagccaaaactccttttctcttgacctctgctctgcatccaaatacccataaagaatgcttctaagcacaggcacaggcacaggtgtgatttttaatatacccagagctgaggagaccttttgtcatcatgactgtcactgtcagtcttttcatcaacataagatcactatcattatcattctgagagacaacactgcttacaggtataataccagtttggagagcagataggttaatctggatagtcttcccgaaagaagcaagccttggaaatggttctcatgaggaaggaaggtgtgaggtgggggtgaagcagggtattgagtgggaggcctagaggagggcctggcgcttggcatctactctgctgtataggctcctaacccctctatggtctcaaggccctctcacctccatggcctaaaacccacctaagttagccatcgtgaacaaatttcccaatagcaggaaagacccagggatgttttctgagctccacccagagcagttaggcccaaaacacagaccttccaggccagcaggcttcagaccaagcagacagcactcaggcttcaagttctcacctgtctgctacctcatgcaggaagcaccatttgggatcttgcaggttcctggaagtagagctacagcagtgcagattggggcaggcggtggggtgtgtgtgtgtgtgtgtgcctttgtgtgtgtgtgtgcctttgtgtgtgtgtctgtgtgtgtctgtgtgtgtctgtgtgactgtgtgtctgagtctgagtctgagtctgagtctgagtctgagtctgagtctgagtctgagtctgtgtctgtgtctgtgtctgtgtctgtgtctgtgtctgagtctgtgtctgtgtctgtgtctgtgtctgtgtctgtgttgtctgtgtctgtgtctctgtgtctgtgtctatgtctgtgtctgtgtctgtgtctgtgtctgtctgtgtctgtgtcgtctgtgtctgtgtctgtgtctgtgtctgtgtctgtgtctgtctgtgtctgtgtctgtctgtgtctgtgtctgtgtctgtctgtgtctgtgtcgtctgtgtctgtgtctgtgtctgtgtcgtctgtgtctgtgtctgtctgtgtctgtgtctgtctgtgtctgtgtcgtctgtgtctgtgtctgtgactgtgtctgtgtcgtctgtgtctgtgtcatctgtgtctgtgtctgtgtctgtgtctgtgtctgtgtctgtgtctgtgtctgtgtctgtgtctgtctgtgtctgtgtcgtctgtgtctgtgtctgtgtctgtgtctgtctgtgtctgtgtcgtctgtgtctgtgtctgtgtctgtgtctgtctgtgtctgtgtctgtctgtgtctgtgtctgtctgtgtctgtgtcgtctgtgtctgtgtctgtgtctgtgtctgtgtctgtgtctgtgtctgtgtctgtgtctgtgtctgtgtctgtgtctgtgtctgtctgtgtctgtgtctatgcctgtgtgtctgtgtctgtgtctgtgtctgtgtctgtgtctgtgtctgtgtctgtgtctgtgtctgtgtctgtgtctgtgtctgtctgtgtctgtgtctgtctgtgtctgtgtctgtgtctgtgtctgtgtctgtgtctgtgtctgtgtctgtgtctgtgtctgtgtctgtgtctgtgtctgtgtcagaggggagtggcagggccaggggtgggagagctctgatccatcttcagcaaagtggggacaggtatagtcagggaggaagcttccaagccacatcctactcctgtgggctccgaggaacccgggcttattctgattttaagttaaggatgaacctgaaatgcacagagctcagcagcctaacctgcagccctagtaggtaatggcagctgtgcttgactttcgaggggagaagcttccatcccactccagtttgaggagccgaagctggccctctctgacccaaggcagaggtgttgggcttctgcatggctagttaacaagctggaatgcataaacaagaaggcttgctctgataactgcctctttgtcttgcctatcatggccccttgctgctcttcactggttctggatctgacttgattgtttgtttgtttgtttgtttgtttgtttgtttttaaagatagactctcactttgtatctctggatggcttcaaactcatagagatctgcccgcctctgcctcttgaatgctggtgtccaagcccagcacacccatcctgatgggattttaattagccaggatagcatacatgtgccagtgcccccctccactaaatgccagcctccccaggtctctggtgagaatgggagtgaggggctgcagccacccatgcctggggcacagaccccatctcctagatcttagataggcaggtggtttggacttacttcttagaacggagtacaattctttcccatgtctcaccctttcaggaaaggaacatgaatacaacatttggggagaagaagctgagtgaaatgggaaataggggttcccggggcaagacagggcccatgggtgatcttcaatattcttctcctctctggggccccagtgttagaactgtgtgattgatggtcatacggcctacactcaggctaataggcggtgcccatgtcactggcactgctcactgggacctccactcctgttactaatggcaagttttagaacacttggaaacagcaacatatcatcactcagagtgacaaagcttgccctgatttgcacgaggccattgacagcatttgtacatataatgtatttagtgaggtgtgcaattagggccgctgtcctgtgcccactgtgcacatcactcagtcagggctgctgtcctgtgccctctgtgcacacctgacagtggtatttataatgatcttccccactgaaacctgaacaatcttgaatacctaaaatacctttgggccccaaggatgtgcattccagtgaggaaccatgggactcccatctattccatgtaacactccatcttaccaagcttgaacacacagggcctatgaaaccaaggttaacagaggaaaaccccttagctgggaaaactgatcacgtctgccactgaagtgccaaagcccaccagatgcgggcagtagaggacccaggggaaattggcccagagagtcccatcagtcaggcatacagaggaatgggggtagggctcttctacagttcatggaggggtcccttcctgtgctgcaccccaagaactttgatgtccccaccttgtgcctgcagctgaagaagatgaaagagaagaagggcctgtaccccgacaagagcatctacacccagcagataggccccggcctgtgctttggggccctggccctgatgcttcgattcttctttgaggtacctggcctatggggaaggggagcaagagcctctggaaatagttaactaacagccacatatggaggctccaagttgtaaattctgtcctcttgaggatggtactcaccatccccagtgtactagcctcactgcctgagtggacaagtcaggccctgactttaggaagcctgcacccaagatgcacgcatgtcctggaaggaaccaggagctggtgcccagcacccaggaggcatggaacagaggaaggcagcaagcctttctgccaccttctcagggtgggctggctgacagaggcaccaaatggctaggacttggtttagggaagggatggaggcccagactgactcattctcctccacaggaatgggattacacctacgtccacagcttctaccactgtgccctggccatgtcctttgtcctgctgctgcccaaggtcaacaagaaggctgggaacgcaggggcccccgccaagctgaccttctccaccctctgctgcacttgtgtctgactatacccccccacacacacacacacaccaggcccctgccttcctgcctggcagtcctgctgtctctcccaaggtacttcctatactttgttatgcggcctgtacatgagaaatggtcttctctacaccccaagagaccagcaggcctgctgcattctgctgagtgctgcttagggacccactggttctgtgttcaccagttgcttcactctgttcaggaaaaaaaagaactttatcccccaaggcctcacaaccataggtgtgcctggcagagaaccctagaccagtaaatacccagcagcatgcagggttatctatttcccaggtcctgcctgtcagaatcgtctgctttccctaggaaactgggattgcccttttaacccctgcccaggctctgaaagctctccacttaggaagctggagccagcgaaccttgcatacccctgcctgagtcccatcccttctgcaggttttcgatcaagccattcacagtaaactcttgatcagccactagtcattagcttcgcctaacatacattctagttcctggaa

SEQ ID NO: 17 - DNA, 19 bp, Artificial Sequence
  Forward Primer
atcgctaccaagaggcgtt

SEQ ID NO: 18 - DNA, 20 bp, Artificial Sequence
  Reverse Primer
cacagcacagacaaaccagg

SEQ ID NO: 19 - DNA, 20 bp, Artificial Sequence
  Forward Primer
tgcgacttcaacagcaactc

SEQ ID NO: 20 - DNA, 20 bp, Artificial Sequence
  Reverse Primer
gcctctcttgctcagtgtcc

SEQ ID NO: 21 - DNA, 19 bp, Artificial Sequence
  Forward Primer
aggttcgttcactcatgga

SEQ ID NO: 22 - DNA, 19 bp, Artificial Sequence
  Reverse Primer
tcgaccagtttagttaccc

(19) United States
(12) Patent Application Publication
(10) Pub. No.: US 2022/0378841 A1
(43) Pub. Date: Dec. 1, 2022

(54) MODIFIED CELLS AND RELATED METHODS

(71) Applicant: CHILDREN'S HOSPITAL MEDICAL CENTER, Cincinnati, OH (US)

(72) Inventors: Douglas MILLAY, Park Hills, KY (US); Yasuyuki MITANI, Shinagawa-ku, Tokyo (JP)

(73) Assignee: CHILDREN'S HOSPITAL MEDICAL CENTER, Cincinnati, OH (US)

(21) Appl. No.: 17/597,359

(22) PCT Filed: Jul. 9, 2020
(86) PCT No.: PCT/US2020/041309

§ 371 Date: Jan. 4, 2022

Related U.S. Application Data
(60) Provisional application No. 62/872,610, filed on Jul. 10, 2019.

(51) Int. Cl. A61K 35/28; A61K 35/33; A61P 21/00

(52) U.S. Cl. CPC A61K 35/28; A61K 35/33; A61P 21/00

(57) ABSTRACT

Some embodiments of the invention include modified cells. Certain embodiments of the invention include methods of using modified cells. Other embodiments of the invention include methods of administering modified cells. Further embodiments of the invention include methods of administering modified cells to treat diseases. Additional embodiments of the invention are also discussed herein.

### CROSS-REFERENCE TO RELATED APPLICATIONS

This application claims the benefit of U.S. Provisional Application No. 62/872,610, filed Jul. 10, 2019 entitled “POLYPEPTIDES, NUCLEIC ACID MOLECULES, CELLS, AND RELATED METHODS” which is herein incorporated by reference in its entirety.

### GOVERNMENT RIGHTS

This invention was made with government support under NIH AR068286 awarded by the National Institutes of Health. The government has certain rights in the invention.

### REFERENCE TO A SEQUENCE LISTING

The instant application contains a Sequence Listing that has been submitted in ASCII format via EFS-Web and is hereby incorporated by reference in its entirety. Said ASCII copy, created on Jun. 29, 2020, is named 2020_07_seq_listing_36821_04049_ST25.txt and is 73 KB in size.

### BACKGROUND

The muscular dystrophies (MD) are a group of inherited muscle disorders caused by mutations in the dystrophin-glycoprotein complex (DGC), which provides stability for the muscle cell membrane. A form of MD is Duchenne muscular dystrophy (DMD) that affects 1 in 3500 boys and is caused by a mutation in dystrophin, which results in severe muscle wasting. Some treatments and treatment strategies exhibit potential, but alternative strategies may be beneficial.

Certain embodiments of the invention address one or more of the issues described above. Some embodiments of the invention include modified cells. Certain embodiments of the invention include methods of using modified cells. Other embodiments of the invention include methods of administering modified cells. Further embodiments of the invention include methods of administering modified cells to treat diseases. Additional embodiments of the invention are also discussed herein.

### SUMMARY

Some embodiments of the invention include a method for administering a modified cell to an animal comprising administering a modified cell to an animal, wherein the modified cell is a modified cell as disclosed herein. In some embodiments, the modified cell expresses a myomaker polypeptide, expresses a dystrophin polypeptide, or both. In other embodiments, the modified cell expresses a myomaker polypeptide, overexpresses a dystrophin polypeptide, or both. In still other embodiments, the dystrophin polypeptide is a microdystrophin or a minidystrophin. In certain embodiments, the modified cell is a modified animal cell, a modified vertebrate cell, a modified mammalian cell, a modified human cell, a modified rat cell, a modified mouse cell, a modified muscle cell, a modified non-muscle cell, a modified myoblast, a modified fibroblast, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell. In yet other embodiments, the modified cell is a modified myoblast, a modified fibroblast, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell. In some embodiments, the modified cell is an MSC cell which expresses a myomaker polypeptide and overexpresses a dystrophin polypeptide. In other embodiments, the administering is parenteral administration, mucosal administration, intravenous administration, depot injection, subcutaneous administration, topical administration, intradermal administration, oral administration, sublingual administration, intranasal administration, or intramuscular administration. In certain embodiments, the administering is an injection or an intramuscular injection. In yet other embodiments, the animal is selected from mammals, primates, monkeys, macaque, rhesus macaque, or pig tail macaque, humans, canine, feline, bovine, porcine, avian, chicken, mice, rabbits, and rats. In still other embodiments, the animal is a mouse, rat, or human. In some embodiments, the animal is in need of treatment of a disease. In certain embodiments, the disease is a disease where the animal's cells underexpress dystrophin, do not express dystrophin, or express a defective form of dystrophin. In still other embodiments, the disease is myopathy, muscular dystrophy, amyotrophic lateral sclerosis (ALS or also called Lou Gehrig's disease), glycogen storage disease type II (also called Pompe disease), rhabdomyosarcoma (RMS), or sarcopenia. In yet other embodiments, the disease is muscular dystrophy.

Some embodiments of the invention include a modified cell as disclosed herein. In other embodiments, the modified cell expresses a myomaker polypeptide, expresses a dystrophin polypeptide, or both. In certain embodiments, the modified cell expresses a myomaker polypeptide, overexpresses a dystrophin polypeptide, or both. In still other embodiments, the dystrophin polypeptide is a microdystrophin or a minidystrophin. In yet other embodiments, the modified cell is a modified animal cell, a modified vertebrate cell, a modified mammalian cell, a modified human cell, a modified rat cell, a modified mouse cell, a modified muscle cell, a modified non-muscle cell, a modified myoblast, a modified fibroblast, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell. In certain embodiments, the modified cell is a modified myoblast, a modified fibroblast, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell. In some embodiments, the modified cell is an MSC cell which expresses a myomaker polypeptide and overexpresses a dystrophin polypeptide.

Other embodiments of the invention are also discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the description of specific embodiments presented herein.

FIG. 1: In vitro and in vivo heterologous fusion of myomaker⁺ MSCs with muscle cells. (A) Schematic of the in vitro fusion study. MSCs were isolated from WT mouse bone marrow, transduced with myomaker and GFP retroviruses, and then co-cultured with WT primary myoblasts and differentiated. (B) Cells were fixed after five days of differentiation and immunostained with a myosin antibody (red). Myomaker⁺ MSCs fused with myoblasts and formed chimeric myotubes (myosin⁺ GFP⁺). (C) Representative muscle sections four weeks after transplantation of MSCs into TA muscles of WT mice, which were injured with cardiotoxin twenty-four hours before transplantation. Sections were stained with a dystrophin antibody (red) to identify myofibers. (D) Quantification of the number of GFP⁺ myofibers per section. Data is represented as mean±SEM, n=4 muscles. ** p<0.01, compared to Empty. Scale bar: 100 μm.

FIG. 2: Myomaker⁺ MSCs fuse with uninjured muscle. (A) Representative muscle sections two weeks after transplantation of MSCs into uninjured TA muscles of WT mice. GFP fluorescence was observed in both myofibers with and without central nuclei suggesting myomaker⁺ MSCs fused with both regenerating and non-regenerating fibers. DMD: dystrophin. (B) Myomaker-expressing MSCs do not readily fuse with cardiac muscle. GFP⁺ myomaker⁺ MSCs were delivered via direct intracardiac injection to Rosa^{mTmG} mice and engraftment was assessed by confocal microscopy three days or fourteen days following transplantation. Myomaker⁺ MSCs (GFP⁺) were detected along the injection sites within recipient hearts (membrane tdTomato⁺) three days post-delivery, and were retained out to fourteen days. Myomaker⁺ MSCs were localized within the needle puncture wound indicating areas of injection, but MSC fusion with cardiomyocytes were rarely detected. The arrowhead denotes a single fusion event, indicated by cytoplasmic GFP within a membrane tdTomato⁺ cardiomyocyte. Scale bar, 100 μm. (C) Quantification of GFP⁺ fibers per section with and without central nuclei after transplantation of myomaker⁺ MSCs into uninjured muscle. n=4 muscles. ** p<0.01, compared to Empty without central nuclei. (D) Tamoxifen regimen efficiently deletes myomaker in satellite cells resulting in lack of fusion. Myomaker^{LacZ/loxp}; Pax7^{CreERT2/+} mice were treated with tamoxifen) (myomaker^{scKO}) for five consecutive days. Controls were vehicle-treated Myomaker^{LacZ/loxp}; Pax7^{CreERT2/+} mice. Multiple muscles were injured with cardiotoxin (CTX) to activate myogenic progenitors and these cells were isolated three days after injury. (E) Tamoxifen regimen efficiently deletes myomaker in satellite cells resulting in lack of fusion. Myomaker expression by qPCR revealed efficient deletion in myoblasts from myomaker^{scKO} mice. (F) Tamoxifen regimen efficiently deletes myomaker in satellite cells resulting in lack of fusion. Control and myomaker^{scKO} myoblasts were differentiated for three days. Control myoblasts fused normally while myomaker null myoblasts failed to undergo fusion. (G) Schematic of transplantation study using myomaker^{scKO} mice. Satellite cell-derived myomaker was deleted through treatment with tamoxifen for five consecutive days. (H) Representative muscle sections two weeks after transplantation of myomaker⁺ MSCs into uninjured TA muscles of myomaker^{scKO} mice. Central nuclei were not observed in myomaker^{scKO} mice demonstrating an inhibition of regeneration. (I) Quantification of the number of GFP⁺ fibers per section with and without central nuclei after transplantation into myomaker^{scKO} muscle. N.S., non-significant. n=4-6 muscles. Data represented as mean±SEM. (J) WT myoblasts fuse more efficiently than myomaker KO myoblasts. Muscle sections two weeks after transplantation of myoblasts into WT TA muscles. Primary myoblasts were isolated from the muscles of WT and myomaker^{scKO} mice and transduced with GFP and dsRed, respectively. Each population of myoblasts was transplanted separately or mixed and transplanted into TA muscles of WT mice, which were injured with cardiotoxin twenty-four hours before transplantation. WT-GFP myoblasts exhibited increased engraftment compared to dsRED-myomaker KO myoblasts. dsRed⁺ unfused mononuclear cells (middle panel—arrowheads) were observed but this was absent after transplantation of WT-GFP myoblasts (left panel). After co-transplantation (right panel) minimal dsRed⁺ (arrowheads) and GFP⁺ dsRed⁺ myofibers (arrows) were detected. n=3-4. Scale bar: 100 μm.

FIG. 3: Evaluation of dystrophin restoration in mdx^{4cv} muscle after heterologous fusion. (A) Schematic of protocol for in vitro fusion study. GFP myomaker⁺ MSCs were co-cultured with primary myoblasts isolated from mdx^{4cv} mice and differentiated. (B) Cells were fixed after five days of differentiation and immunostained with a dystrophin antibody. Dystrophin expression was observed at the membrane of myotubes fused with myomaker⁺ MSCs but not in unfused mdx^{4cv} myotubes. (C) Muscle sections two or six weeks after transplantation of GFP-expressing myomaker⁺ MSCs, or WT-GFP myoblasts as a control, into uninjured TA muscles of mdx^{4cv} mice. One group of mdx^{4cv} mice was treated with trichostatin A (TSA) as a means to enhance reprogramming. Fusion (GFP⁺ myofibers) was observed at both time points but GFP⁺ dystrophin⁺ myofibers were detected only in the myoblast-transplanted muscle. Dystrophin⁺ myofibers in myomaker⁺ MSC transplanted muscle are not GFP⁺ and are likely revertants (arrowheads). (D) Quantification of the number of GFP⁺ fibers per section in the muscles two weeks after MSC transplantation. n=4 muscles. * p<0.05, compared to Empty. Scale bars: B, 25 μm; C, 100 μm. Data represented as mean±SEM.

FIG. 4: Myomaker-mediated heterologous fusion of CBSCs and TTFs with mdx^{4cv} muscle and dystrophin reprogramming. (A) Schematic of protocol for in vitro fusion study. CBSCs were isolated from tibias and femurs of Rosa26^{mTmG} mice and infected with myomaker retrovirus. TTFs were isolated from tail-tips of WT mice and retrovirally transduced with myomaker and GFP. Cells were co-cultured with mdx^{4cv} primary myoblasts and differentiated. (B) Co-cultured cells were fixed after five days of differentiation and immunostained with myosin and dystrophin (DMD) antibodies. Myomaker⁺ CBSCs and TTFs fused with myoblasts and formed chimeric myotubes, which displayed dystrophin expression at the membrane. (C) Quantitative RT-PCR for myomaker in MSCs, CBSCs, and TTFs demonstrates myomaker expression is similar after infection of non-muscle cells. Each cell type exhibits higher myomaker levels than differentiated myoblasts (DM). GM: growth medium. (D) Representative muscle sections two weeks and ten weeks after transplantation of CBSCs and TTFs, respectively, into uninjured TA muscles of mdx^{4cv} mice. mTomato⁺ or GFP⁺ myofibers were observed in the myomaker-cell-transplanted muscles indicating fusion, however dystrophin was not detected in them. Only revertant fibers (arrowheads) were observed in mdx^{4cv} mice. (E) Quantification of the number of mTomato⁺ or GFP⁺ fibers per section. n=3 or 4 muscles. * p<0.05, compared to Empty. Data represented as mean±SEM. Scale bar: B (first and third panels) and D, 100 μm; B (second and fourth panels), 25 μm.

FIG. 5: Non-dystrophin in vivo reprogramming induced by heterologous cell fusion. (A) Schematic of protocol for detection of in vivo reprogramming. CBSCs were isolated from Myl1^{Cre/+} mice, transduced with myomaker and GFP retroviruses, then transplanted into CTX-injured TAs of Rosa26^{tdTomato} mice. (B) PCR for Cre demonstrates that myomaker⁺ Myl1^{Cre/+} CBSCs do not express Cre. TTFs from β-actin-Cre mice were used as a positive control. (C) Whole mount fluorescence image of Rosa26^{tdTomato} muscles transplanted with either myomaker⁺ Myl1^{+/+} CBSCs or myomaker⁺ Myl1^{Cre/+} CBSCs four weeks after transplantation. (D) Representative sections of TAs from Rosa26^{tdTomato} mice after transplantation. Both myomaker⁺ Myl1^{+/+} CBSCs and myomaker⁺ Myl1^{Cre/+} CBSCs fused (GFP⁺ myofibers), however tdTomato expression (reprogramming) was only observed in muscles transplanted with myomaker⁺ Myl1^{Cre/+} CBSCs. Scale bars: C, 1 cm; D, 100 μm.

### DETAILED DESCRIPTION

While embodiments encompassing the general inventive concepts may take diverse forms, various embodiments will be described herein, with the understanding that the present disclosure is to be considered merely exemplary, and the general inventive concepts are not intended to be limited to the disclosed embodiments.

Some embodiments of the invention include modified cells. Certain embodiments of the invention include methods of using modified cells. Other embodiments of the invention include methods of administering modified cells. Further embodiments of the invention include methods of administering modified cells to treat diseases. Additional embodiments of the invention are also discussed herein.

Myomaker Polypeptides and Myomaker Nucleic Acid Molecules

Some embodiments of the invention include compositions comprising the myomaker polypeptide, the myomaker nucleic acid molecule, or both, cells comprising the myomaker polypeptide, the myomaker nucleic acid molecule, or both, or using the myomaker polypeptide, the myomaker nucleic acid molecule, or both. In some embodiments, the myomaker polypeptide is the myomaker protein disclosed in WO 2014/210448 A1, which is herein incorporated by reference in its entirety. In other embodiments, myomaker polypeptide is the myomaker protein disclosed in Table 10A of WO 2014/210448 A1. In some embodiments, the myomaker polypeptide is the myomaker protein disclosed in WO 2018/152103 A1, which is herein incorporated by reference in its entirety. In other embodiments, myomaker polypeptide is the myomaker protein disclosed in Table 2 of WO 2018/152103 A1. The term “myomaker polypeptide” encompasses “wt-myomaker polypeptides” (i.e., myomaker polypeptides found in nature without any purposely human-made modification) and “mutant myomaker polypeptides” (e.g., with one or more modifications made to a wt-myomaker polypeptide). Nonlimiting examples of wt-myomaker polypeptides are found in Table 10A of WO 2014/210448 A1, in Table 2 of WO 2018/152103 A1, or in Table 1A. In other embodiments, the myomaker polypeptide has at least one amino acid modification relative to a wt-myomaker polypeptide. A wt-myomaker polypeptide can, in some embodiments, be a myomaker polypeptide from any animal including but not limited to a mammal, a rat, a cat, a rabbit, a human, a cow, a chicken, a turkey, a monkey, a tree shrew, a dog, a pig, a shrew, an elephant, or an opossum. Table 1A provides nonlimiting examples of wt-myomaker polypeptides and Tables 1B and 1C provide nonlimiting examples of related nucleic acid sequences (including start and stop codons).

TABLE 1A

| Source | Polypeptide sequence |
|---|---|
| Human | MGTLVAKLLLPTLSSLAFLPTVSIAAKRRFHMEAMVYLFTLFFVALHH |
| | ACNGPGLSVLCFMRHDILEYFSVYGTALSMWVSLMALADFDEPKRST |
| | FVMFGVLTIAVRIYHDRWGYGVYSGPIGTAILIIAAKWLQKMKEKKG |
| | LYPDKSVYTQQIGPGLCFGALALMLRFFFEDWDYTYVHSFYHCALAM |
| | SFVLLLPKVNKKAGSPGTPAKLDCSTLCCACV (SEQ ID NO: 1) |
| Dog | MGTLAAKLLLPTLSSLAFLPTVSIAAKRRFHMEAMVYLFTMFFVALH |
| | HACNGPGLSVLCFMRHDVLEYFSVYGTALSMWVSLMALADFDEPKR |
| | STFVMFGVLTIAVRIYHDRWGYGVYSGPIGTAVLIIATKWLQQMKEK |
| | KSLYPDKSVYTQQIGPGLCFGALALMLRFFFEDWDYTYVHSFYHCAL |
| | AMSFVLLLPKVNKKAGSAGPPAKLDCSTLCCACI (SEQ ID NO: 2) |
| Pig | MGTVMAKLLLPTLSSLAFLPTVSIAAKRRFHMEAMVYLFTTFFVAFY |
| | HACHGPGLAMICFLRLDILEYFSVYGTALSMWVSLMALADFDEPKRS |
| | TFVMFGVLTIAVRIYHDRWGYGVYSGPIGTAALIIAAKWLQQMKDQR |
| | RLYPDKSVYTQQIGPGLCFGALALMLRFFFEEWDYTYVHSFYHCALA |
| | MSFVLLLPKANKKAGSAGPPAKLDCSTLCCACI (SEQ ID NO: 3) |
| Mouse | MGTVVAKLLLPTLSSLAFLPTVSIATKRRFYMEAMVYLFTMFFVAFSH |
| | ACDGPGLSVLCFMRRDILEYFSIYGTALSMWVSLMALADFDEPQRSTF |
| | TMLGVLTIAVRTFHDRWGYGVYSGPIGTATLIIAVKWLKKMKEKKGL |
| | YPDKSIYTQQIGPGLCFGALALMLRFFFEEWDYTYVHSFYHCALAMSF |
| | VLLLPKVNKKAGNAGAPAKLTFSTLCCTCV (SEQ ID NO: 4) |
| Opossum | MGTLVTKLLLPTISSLAFLPTISIAAKRRFHMEAMVYLFTMFFIAIYHA |
| | CDGPGLSVLCFMRYDILEYFSIYGTALSMWVSLMALAEFDEPKRSTFV |
| | MFGVLTIAVRIYQDRWGYGVYSGPIGTAVLIIATKWLQKMKEKKGLY |
| | PDKSVYTQQIGPGFCFGALALMLRFFFQEWDYTYVHSFYHCSLAMSF |
| | VLLLPKVNKKAGNAGTPAKLDCSTLCCACI (SEQ ID NO: 5) |
| Zebrafish | MGAFIAKMLLPTISSLVFVPAASVAAKRGFHMEAMVYFFTMFFTAIY |
| | HACDGPGLSILCFMKYDILEYFSVYGTAISMWVTLLALGDFDEPKRSS |
| | LTMFGVLTAAVRIYQDRLGYGIYSGPIGTAVFMITVKWLQKMKEKKG |
| | LYPDKSVYTQQVGPGCCFGALALMLRFYFEEWDYAYVHSFYHVSLA |
| | MSFILLLPKKNRYAGTGRNAAKLNCYTLCCCV (SEQ ID NO: 6) |

TABLE 1B

| Source | cDNA nucleic acid sequence |
|---|---|
| Human | atggggac gctggtggcc aagctgctcc tgcccaccct cagcagcctg gccttcctcc ccactgtcag |
| | catcgcggcc aagaggcggt tccacatgga ggccatggtc tacctcttca ccctgttctt |
| | cgtggcgctc caccatgcct gcaatggacc cggcttgtct gtgctgtgct tcatgcgtca cgacatcctg |
| | gagtatttca gtgtctacgg gacagccctg agcatgtggg tctcgctgat ggcactggcc |
| | gacttcgacg aacccaagag gtcaacattt gtgatgttcg gcgtcctgac cattgctgtg cggatctacc |
| | atgaccgatg gggctacggg gtgtactcgg gccccatcgg cacagccatc ctcatcatcg |
| | cggcaaagtg gctacagaag atgaaggaga agaagggcct gtacccagac aagagcgtct |
| | acacccagca gataggcccc ggcctctgct tcggggcgct ggccctgatg ctacgcttct |
| | tctttgagga ctgggactac acttatgtcc acagcttcta ccactgtgcc ctggctatgt cctttgttct |
| | gctgctgccc aaggtcaaca agaaggctgg atccccgggg accccggcca agctggactg |
| | ctccaccctg tgctgtgctt gtgtctga (SEQ ID NO: 7) |
| Dog | atgggga cgctcgcggc gaagctgctc ctgcccaccc tcagcagcct ggccttcctc cccaccgtca |
| | gcatcgccgc caagcggcgg ttccacatgg aggccatggt ctacctcttc accatgttct |
| | tcgtggcact ccaccacgcg tgcaacgggc ccgggctatc ggtgctctgc ttcatgcgcc |
| | acgacgtcct ggagtacttc agcgtctatg ggacggcact gagcatgtgg gtctcgctga |
| | tggcactggc tgacttcgac gaacccaaga ggtcgacttt tgtgatgttt ggcgtcctga ccatcgccgt |
| | gcggatctac catgaccgct ggggctacgg ggtgtactcg ggccccattg gcacggctgt |
| | cctcatcatc gccacaaagt ggctgcagca gatgaaggag aagaagagtc tgtacccgga |
| | caagagtgtc tacacccagc agataggccc tggcctctgt tttggggcac tggcccttat gctgcgcttc |
| | ttttttgagg actgggatta cacctatgtc cacagcttct accactgtgc cctggccatg tccttcgtcc |
| | tcctgctccc caaggtcaac aagaaggctg gaagcgcggg gccccctgcc aagctagact |
| | gctctaccct ttgctgtgct tgcatctga (SEQ ID NO: 8) |
| Pig | atgg ggaccgtcat ggccaaactg ctgctaccca cgctgagcag cctggccttc ctccccacgg |
| | tcagcatcgc tgccaagcgg cggttccaca tggaggccat ggtctatctc ttcaccacgt tcttcgtggc |
| | gttctaccac gcctgccacg ggccgggcct ggctatgatc tgctttctgc gccttgacat cctggagtat |
| | ttcagcgtct acggaaccgc cctgagcatg tgggtctcgc tgatggcgct ggctgacttc |
| | gacgagccca agaggtcgac tttcgtgatg tttggcgtcc tgaccatcgc cgtgcggatc |
| | taccacgacc gctggggcta cggcgtgtac tcgggcccca tcggcacggc cgccctcatc |
| | atcgcggcca agtggctgca gcagatgaag gaccaacggc gcctgtatcc agacaagagc |
| | gtgtacacac agcagatagg ccccggcctc tgcttcgggg cgctggccct catgctgcgc tttttcttcg |
| | aggagtggga ttatacctac gtccacagct tctaccactg cgccctggcc atgtccttcg tcctgctgct |
| | gcccaaggcc aacaagaagg ctggaagcgc agggccaccc gccaagctgg actgctccac |
| | cctctgctgt gcttgtatct ga (SEQ ID NO: 9) |
| Mouse | atgg ggacagttgt agccaaactg ctcctgccta ccctcagcag cctggccttc ctcccgacag |
| | tgagcatcgc taccaagagg cgtttctaca tggaggccat ggtctacctc ttcaccatgt tctttgtggc |
| | gttctcccat gcctgtgatg ggcctggttt gtctgtgctg tgcttcatgc gccgtgacat tctggagtac |
| | ttcagcatct atggaacagc cctgagcatg tgggtctccc tgatggcact ggccgacttt |
| | gatgaacccc agagatcgac cttcacaatg cttggcgtcc ttaccatcgc tgtgcggact tttcatgacc |
| | gctggggtta cggggtatac tccggtccca taggcacggc caccctcatc attgctgtaa |
| | agtggctgaa gaagatgaaa gagaagaagg gcctgtaccc cgacaagagc atctacaccc |
| | agcagatagg ccccggcctg tgctttgggg ccctggccct gatgcttcga ttcttctttg aggaatggga |
| | ttacacctac gtccacagct tctaccactg tgccctggcc atgtcctttg tcctgctgct gcccaaggtc |
| | aacaagaagg ctgggaacgc aggggccccc gccaagctga ccttctccac cctctgctgc |
| | acttgtgtct ga (SEQ ID NO: 10) |
| Opossum | atggg gactcttgtt accaagttgc ttcttcccac aatcagcagc ctcgcctttc tccccaccat |
| | cagcatcgct gctaagagga gattccacat ggaagccatg gtctacctct tcaccatgtt cttcatagca |
| | atatatcatg catgtgacgg gccaggctta tcagtgctat gcttcatgcg ctatgacata ctggagtatt |
| | tcagcatcta tgggacagca ctgagcatgt gggtgtcatt aatggcactg gcagagttcg |
| | atgaaccaaa aaggtcaacc tttgtaatgt ttggcgtgtt gactattgcc gtgaggatct accaagaccg |
| | gtggggatat ggggtatact cggggcctat tggcacagct gtccttatca ttgcaacaaa |
| | atggctgcaa aagatgaaag agaagaaggg tctgtaccct gacaagagtg tgtacaccca |
| | acagataggc cctggtttct gttttggagc gttagcactg atgctgcgtt tctttttcca ggagtgggat |
| | tacacctatg ttcacagctt ctaccactgt tcactagcca tgtcctttgt cttgctgctg cccaaggtaa |
| | acaagaaagc tgggaatgct gggacacctg ccaaattgga ctgttctaca ctctgctgtg cttgcatctg |
| | a (SEQ ID NO: 11) |
| Zebrafish | atgggag cgtttatcgc caagatgttg ctgcccacta ttagcagttt ggtgtttgtg cctgcagcca |
| | gcgtggctgc aaagaggggc ttccacatgg aggccatggt ctatttcttc acaatgttct tcaccgcgat |
| | ttaccacgca tgtgacggtc cgggcttgtc cattctctgt ttcatgaagt atgacattct ggagtacttc |
| | agcgtgtacg ggacagccat ctccatgtgg gtcacgctac tggcgcttgg ggatttcgat |
| | gagcccaaac gctcttcgct caccatgttt ggggtgttga ccgcagctgt gaggatttac |
| | caggaccgac tgggctacgg catttactcc ggccccatcg ggacagctgt ctttatgatc |
| | acagtcaaat ggttacagaa aatgaaggaa aagaaaggcc tttatccaga caaaagtgtt |
| | tacactcaac aagtgggccc agggtgctgc ttcggtgctc ttgctttgat gcttcgcttc tattttgagg |
| | agtgggacta cgcttatgtt cacagtttct accacgtgtc tctggccatg tcctttattc tgctgctgcc |
| | caagaagaac cgttatgctg gaacgggacg taacgcagcc aaactcaact gctacaccct |
| | ctgctgctgt gtatga (SEQ ID NO: 12) |

TABLE 1C
(exons in lowercase)

| Source | Genomic nucleic acid sequence |
|---|---|
| Human | caagtgtgagctggggagggcaggggctcagagccgggctgggcgcagcatcagacacaa |
| (+ strand) | gcacagcacagggtggagcagtccagcttggccggggtccccggggatccagccttcttg |
| | ttgaccttgggcagcagcagaacaaaggacatagccagggcacagtggtagaagctgtgg |
| | acataagtgtagtcccagtcCTGCGGGGGGCAAGCGGTCAGTCTGGGGCCTCAGCCCCCT |
| | CCCCGAGGCTCCTCCCTCTCCAAGACCCAGCAGAGCCCCTTCAGGCCCCCGCCTCTGCCA |
| | GGGCACTGGGACACCTGCAGGAAGCCTCCCCCACGGTCGCGCTCACAGTGGTTTTTCTCT |
| | CCACCTAAACCCAGAGCAGTGAGGGCCTGTGCCATCCTCCAGGCTGCACTCCTTCCTTCT |
| | TCCCCATCCCCTCTCTCTGCTGTCCTTCTCTTCCTCCATCCTTCTCTCCCTCCTACCCTC |
| | CCTCCCTCCATCTCCCCCTCTTTTCTCTCCTTATCCCTCTTCCCCTGTTCCTCCCTCCCT |
| | CCTCCACTTTCTCCCTCCTTCCTTCCCTGTCTCCTCCCCTCCCTCCCTCCCTCCTCCAGG |
| | TGTTGGGCACCTGCCCCAGGCGTCTCCCAGGCTGTGCTGCCGTCTGAGATGCCAGCTGTC |
| | TGTAGGCAGCCAGCTTTGGTCTCTGTGACCTCCAGGTCCACACAGGCCATGGTGCTGGTG |
| | GTGCTGGGGACGGCATTGCCCCCGACATAGCCCTGGGAGGGGCTAGTGAGCAGGGACTAA |
| | TACCAGACTTTGGCCTGGGGCTGTCAGAGTCCCCCCAGCGTGGGCACAGCCCTGGTATCC |
| | CAGCTGAGCAGAGCCATGCCGAGTGGGCTCTGGGGCACAGGACACCTCCCCGCTGGGCTT |
| | GGTACctcaaagaagaagcgtagcatcagggccagcgccccgaagcagaggccggggcct |
| | atctgctgggtgtagacgctcttgtctgggtacaggcccttcttctccttcatcttctgt |
| | agCTGTGAGGACAGGAGGCCACAGCAAAGCTTTTAGGTCACAGCACTGGGGAACGCCCCT |
| | CCCCAAACCAGCCCGAGAGCTGGCCCTGCACAGGCTCACCCCAGCCCTCTCCCGGCAGGA |
| | GAGGAGGCTCAGGAGCCTCCTGCCGCACCCAGCCTCAGATGGCTTCTGCTGGACAGGGCC |
| | CTTCACGGTGCGACCCAGCAGAGACCCCAGCCTGGATGGCTGGGAAGGAAGCCACTGGGC |
| | CATGTGCCCCACAAAGACCCCGCTGCCCTCCCGCCTCTTTGAGATGTAACAACGCCACCC |
| | TCGCATGTCTCCTCCTCCCTGGAGGGGAGCTCTGGGGGGACTAGACTCCATGATTGCTTA |
| | CCAAGGAAAGTACTGGAGTACTTGGGACCTGCCAGCCCAGTGTGGCCCATGGGGATGGCA |
| | CTTGTGGTGATCCCTGAGCCATGGACAAGCATCGTTTGCTTTCCTAGTTAAAGGACCTAT |
| | CTCACTCTTCATTAGACAAACTTGGCCAGCACTGCTTCTCAGGTCCCAGTGCTTAGGAAG |
| | GCTCGCGTGGGCGTTTCCACTTACAGAGGGGTTTGCATTCCGAGGAAGATGCGGGAAGTG |
| | TGGGGCCACATCCCTGGAGCCGGCCTTGTGTTTTCTAGGCCACTTCACATGGAGTCTATT |
| | TGGGATTTTCAAGGGCAGTTGTTTCCTGGAATGAGGGTGGATTTTTCTCCCTGAGCCTGG |
| | TCCCCTCTTGGGAGGGGCTGGGGAACGACAGCCTTGTTGGGGAGGAAGGAGGGAGGGTTG |
| | GGTGATGGCGGCCTCGGAGTGGGGCCAGACCCGTGGGGGTACACTCAGGAGGCTATAGAT |
| | TTCAGTGGAATCAACTGTTAGACACACAGCGTGTGGCACAAGCCCCTGGGGGTGGGGGCA |
| | GCACCCCATAACTGCACCCATTGCTGAGTGGCCTATGCAAAGAGCACAAAGAGCCTTATG |
| | CTGGGTCAGGTCAGGTTTTGCCACCCAGTGAATTATGAATTGATGCCCGGCTTTCCATTT |
| | TCTGGAATTCCATTGCCAACAAGGAATTGAGCACCTGCAGTCCTGCAGTGGCCTGAAGAC |
| | AGCTGGACCGTGTGACCCTGGGTGCGGTGGTCAAGGCTGCCAGCCCACCTCTGGCCAGCC |
| | CTGCAGTAGTAACACCAGGGAGAAGAGAGGTGCCTGCCCCAGGTCACACAGTGGGCCTGG |
| | CACTATTGAAAGGGCGCCATCACCCAACCCTCCCTCCTTCTTCCTCCCGGGCTGCCATTG |
| | CCCAACCCCTCCCAAGAGGGGACGAGGCTCAGGGAGAAAAATCCACCCTCATTCCAGGAA |
| | ACAATTGCCCTTGAAAATCCTAAATAAACTCCATACTAAATGGTCTAGAAGACAACAATT |
| | TGAGCCCCAGATGCGGGGAGGCGGGCAGCCCATCCTCGGCTCCTGTGGCTGGATCTGCAG |
| | CCTGAGGGCCTTGGCAGTCTCGTGGCTCTTGGTGGGAAACACAGCAGTGAATTCTCTTCT |
| | GGGCAATTACAGTTCAGCCCAGTTCAGACCTGGCCAAGACCAGCGGGAGGAGCAACCTTC |
| | AGGGGCAGAAGGAGGCGAGAGGCGGGTGGCCAGGACCCAGGGCCCCAGCACGCTCCTTCC |
| | TGCCACCCACCTTGGTCCAGCCCACTTATGCCCAGCGCTCCCTCTCTCCCCACCAGGTGA |
| | CTCCCAGGGGCCTCCTGGGTCAGCCCAGGATTAGTGCTGCTTCCTCAGGTTGCAGACAGA |
| | AAGCAGGTCCTCTGTCTCCTGCTCAAAAAGTCAAGTCCAGCCAGGCGTGGTGGCTCATGC |
| | CTGTAATTCCAGCACTTTGGGAGACTGAGGCAGGCAGATTACCTGAAGTCAGGAGCTCAG |
| | GACCAGCCGGGCCAACGTGGTGAAACCCCATCGCTACTAAAAATATAAAAATTACCTGGG |
| | CGTGATGGCATGCGCCTATAATCCCAGCTACTCGGGAGGCTGAGACAGGAGAATCGCTTC |
| | AACCCGGGAGGCGGAGGTTGCAGTGAGCCAAGATGGCGCCATTGCACTCCAGCCTGGGTG |
| | ACAAGAGCAAAACTCCGTCTCAAAAAAAAAAAAAAAAAAGTCAGGTTCTGGCCCCGCCAC |
| | TGCCCTGCCATGACGTCCTGTTAAGTTGCTGAGGCCTCCATGCTTTGGTTCCTTCATAGG |
| | CCAAATGGCAAATCAGTCCCATGCTCCTTGGCTGTGGGGAGGATTGGGACGGGCTTTGCA |
| | AGCTGCCCACCAGAACTCGAGCGCTCTCCCCACAGCCGTGGGCCCTCCTGCACTGAGAGC |
| | TGCCCTCTGTCTTGCTGGGTGTCCTGCGGCTCTGGCCGGGGCTGGCAGTGTGGCTGGGCT |
| | GGACCAGGCCAGGTCCTCTCTTGGCACTTGAAACTGACCCTGAGACTTCAGGTCCACTCC |
| | AAAGAGGTGAAATGCAGCACAGGGATGTTCAGGCGGTGCCTGGGCTGCTGCAGGCCTGGA |
| | GAGCAGGCTCAGGCTGAAGCCTGCTGGCTCCCCAGGTCTGGGAGACCCTTGCAAGGGTGA |
| | GCTCCCTCCTGCTCTGGGGTCCCAGGAGATGCCCCGGGTCTATTTTTCCCTAAGATCCCT |
| | CTTTAGCTTGGGCGAGTTTGAGTGGGGTTTGGTCCCTGAGCCAGGAGGGTCTTGGTAGGA |
| | CGGAGAGAGCAGGGAGCACTGAAGACCACGTGAGGGCCTTGCTGCTCTGCAAGGGGCTGT |
| | CTGTGCTAGAAGGTCTGGCCCAGGCTGCCTCACTGTCATACCACACTCTCCCTCCTGGCT |
| | AGAACCAAGCTCGAGGCTCACTCCCTCCAGGAAGTCTTCCCAGATTACCCCAGGCCATTT |
| | TCCAAGTTGATGTTGCATCTCTAAAGCAGCTGGTAGTAAGAGCGGTGATGAGAGTGATAA |
| | CAAATAGCTCTTATGTGCGGAGCACATTGGAAGCCAGGCTCCATGCCAGGACTTCAGGTG |
| | CCTGATCTCAGTGAGTCTTTGAACCACCCCATGAGACAGGCAGGGGGCTGTAATGACAAC |
| | ACCTGCTTTACAGGTACGGGCGTGGAGGTGAGACATTGGGTAACTTGGGCTCAGTCTGGA |
| | GCTGGTGAGTACAGACAAGCGTCACACACAGTCTACACAGCCGGAGCACCTCATGGCTAT |
| | TTTCTACGTGGTTTTGCTGAATTCCTGCATCCACCCATTTGCCTATGAGGGCAGGAGGTA |
| | AATGAAGATCCGAGGCAGGAGGAGTCAGACAGGGGAGAGGTGACGGGCCTCCTGGGTCCC |
| | CGTTCATCGAGGCTCGCGCAGTACGCACccactttgccgcgatgatgaggatggctgtgc |
| | cgatggggcccgagtacaccccgtagccccatcggtcatggtagatccgcacagcaatgg |
| | tcaggacgccgaacatcacaaatgttgacctcttgggttcgtcgaagtcggccagtgCTG |
| | GAGGGGCCAGGGAGACACAGGGGGAGGTGAGTGGTCTCTCTTGCTCCTCCTGGCTACCCC |
| | CCCACCCCCCAGCCCCCAGGAGGCATCCTGTAGATGCCCTCTCTCGGTGTCCCCTCAGCC |
| | AGCGAGACCCTGAGGCCCAGCCTGGTCATGGAGGGGTCTGAATTCCAGCCAGTTTGAGAG |
| | GACAGGCAGCCTGCTGCTTCCCCATGGACACAGCAGCTTGGATTGTGCTCCCAGCACCTC |
| | ATTTTAATAAACAGACCACAGCTGGTTGTGGTGGCTCAGGTCTGCAATCCCAGTGCTTTG |
| | GGAGGCAGAGGCAGGAGGATCGTCTGAGACCAGGAGTTCAAGACTAGCCTGGGCAACATA |
| | GCGGGACCCCCATCTCCACAAAAAATTCGGTGGGTGTCGTGGTGCATGCCTGTCATCCCA |
| | GCTACTTGGGAGGCTGAGGTGGGAGGATGGCTTGAGGCTGTGAGTTCGAGGCTGCAGTGA |
| | GCCGTGTTTGTGCCACTGTACTCTGGCCTGAGTGACAGAGTGAGACCCTGTGGCTAAAAA |
| | TCAATAATCACTATGCAAAGTGAATAGGATCGAATCTATCCCATAGGATCACAGGACAAA |
| | GACACTAAGATTCAAGAGAAGAAATGAAGCCCCTCACAGGCCCGGTTAGATGGCAAGGAG |
| | CCTCAGGTCATGGGGACCTTGCCACAGACAACAGTTACGTGGAAAAAAACATGGTGGGAA |
| | AGGGGGCTTATGAACAGTCCCGTCTTCCAGGCTGGATATCACCCGTGTGTGTGGATGTTT |
| | GTATGACAGTCTGGGAAGCCAACCCCCCTGAGCAGTGAACAGCGGTCCTCCCAGGGAAGG |
| | AGTGACGGGAGGGAGCCCTTTCACTTTTTCCTTTGTATGCCTCTGCTGTTGAAATGTGTC |
| | ACAACAAGCTTTTACTAAATGAGTCATTTTAAAAGGATATAAAAAATCGGCATCAGGGCA |
| | TTTAAGAGGTGCATATTCTTTTTCATAGATTAAGCACAACCCTGAAACCCAGACAAGGGA |
| | AGACATTCCTGGGGCTGGGAGTGAGTGGGGATAGAGGGCTGCAGCGGGACTGGTTTGAGG |
| | CTGGGTGTGCGGACACTGGGGAGCCGGTCCTTGTCCGCAAGGCTTGTCTGCAGGGGTTGA |
| | CCACTCACccatcagcgagacccacatgctcagggctgtcccgtagacactgaaatactc |
| | caggatgtcgtgacgcatgaagcacagcacagacaagccgggtccattgcaggcatggtg |
| | gagCTGCCAGAAAACCCACAGGTGGTCACAGCACAAAGAGGCCAGAGCTGGTCCCCGAGC |
| | CACGGCCCCCAGAGTGCCAGGTCACTTGCTGGCTGTGAGAAGTCACTTTGGCGAGTCACT |
| | TAATGACTGTGTGCCTCAGTCTCCCCGTCTGAAAAATGGGGGTACTGCCGAGCACTCCCG |
| | CAGAGGGTCCTGTGGGGATTAAGTGGCACATGCCAGCGAGGTGTTTAGGGGCTGGGGTGT |
| | GCCAAGGGTTCACTCAATGTCACCTCAGCAGAATTCGCTCATCTGCACTGGCAGGACTGG |
| | GCGGAGACTGAGTGGTCACTCAGGTGAAGCCCGCTTAGGTGGGGCGGTCTCCGGGAGGGA |
| | CCCTACACGGCTCTCCCCGGACCTTCAGCATCTGTGCTTCCTTGAAGCACACAGCTGCGT |
| | GTTCACTCGCCAATCTTTGGATGTGAGGTCAGAGCCTCTCTGGGGGCTCCTTTGCTCTTT |
| | GGGGGCTCCTGGGGCCTTCTCTTGCACAAATTACCCCTCTGATGACTGGTCTACACTGCA |
| | GCAGCGTTCTCAGGCTTGAGTGGGCATCAGAACGCCTGGGGCCTTGTTTAGACACAGGTT |
| | ACTGAGCCCTGCCTAGGGTTGCTGATTAGGGAGGGCTGGGTTGGGTAGAAAATGTGCATT |
| | TTGAACACATTCCCTGTGGCACTGCTGAGGCTGGCAGGGCCCACACTGAGAGCCGGGCTG |
| | TAGCTCCTGGTTTCTGTTGCCTTAACGTGGACGAAGATCTCTGAGACCCCCTTGCAGAAG |
| | CTGAACACAGCCCCCTAGGCTCATCCATCTCTGCCCTATACTCTCGTCGTCGCCTCCCCA |
| | ACACCCACTTTCATGGCAATTTTTAAGGCAAAAGGCTTATAGGGAGTGTTTTCAAAGCAG |
| | TCAACTACTTTTCTACGGAAAACAACTCTCTCTCCTTTTGCATTCGCATTTCATCATTTT |
| | AGGTAATATTTAATTACATGACATAATTATTTTGACAGGTTCAACTGGCACAAACAAGCT |
| | TGGGAAACAGCACGGTGGACTCTTGGTCAGCCCAGCTCAGCGGGAGGAGCAGGCGTGCTG |
| | GAAAGCAGCCCGTGTCTGGAGGCGACAGGGACAGCACAGAGGGAGCGGGGGCCCTGGGTG |
| | ATCTGGGGGGCAGGCAATTCGGGGTCAAAGTGGAGTGCTTCTACTGATGGCAATTGTACA |
| | CGGCCTAAAGTGACGGTGCACCTAGGAGGCATTAATAGGGATCCAGCATCTAAAATGAGG |
| | GAGGCGGCGGTCCTGCTTCTCTCTGTTCTTGTCAGGCTCATCCAGAAGACTATGCCGAGC |
| | TCTGTGTGGTGCACCTTTCTTGAAGTGAGACTGGGAAAGACGCGGCTAGAGGAGGGTGAC |
| | CAGCGGTGGACATGACTGTTTACCTTGGGGACAGGGAAGCTTCAGGAGGGGCCTGATCAA |
| | GGTGCTTACACCTCTGTGGGAAAGAGGAGCGAGGAAGACTCCGGCCCTAGGCTGTTCTCC |
| | TGTTCTCCTGGCTTCTTCCCATCCCCCACCCCAGCCCCATCACCTGCTGTCTGTGTGCCT |
| | CAATGTAGCACAGATGGTCATGTGTGATTAAGGCATTCACTGTGAGATTGTGATAAGGCC |
| | TGTGCCCTTGCCCTGCCAGGAGCAGGAATGGCTCTGTCTGGTCCCAGTTGCATGGACGGC |
| | TCCCAGCATAGAGTGCTTGCTGCATGTGTTCAGGGAGGGGGACGCCAGGCTCTGAGAATT |
| | CTAAAGGACAGCCAGCTCACCCTGGGGACCCAGAGCCTCTGCCACTAGGCCCTTGGCTCC |
| | TCCCAATGGTGGGAACTTAGCTCCATTCGACAGATGGGGAAAGTGAACTTCAGAGCAGCA |
| | CTGCCTGCCCAAAGAGGTGAAACAGAGCAGTGCTTGGCACCTGGCCACTTCCTCCCATCC |
| | TGCAGTGCAGGGGGCAGACCTGGCCCAGCCGGGGCACTGGTGGGGTGGGTGCGGCTGAGG |
| | GCCTGGGGGGTCAGAGCTCAGGCTCGGGGAGTCTGACTTTGCAGATGTTCCCAGTGGGGG |
| | CTCAGGTGAGTGGCTGTCGGGGGGGGGCCTCCTCTGTTGTGTGGGGACAAGCACACTGTC |
| | TCCGTGGGGTTTGCACCCATAGCAAGGTGTCCGGCACAGAGATGGAGATTGTCACGGGAG |
| | GGGCCTGATTGGAAGGGAAGGGACGCCATGCGGGTGGCAGAACTTTGGGAGGGACTGAGT |
| | GTGGCTTTGAGTTCAGAAGACGTTTGTCACAAGAGGCAGCTGCCCCTGCCACTCTGGGTG |
| | GGGCAGGGTGGGGCCTCTGAGACCAGTGCAGAGGCAGCTGCGGGGCCAGCCTAGGCCCAG |
| | GCAGGGAGGTGTGGCCTGGTGGGTGCTTGTGGTTTGCTGGGCTAGGTCTAACAGGAGCCT |
| | TGAGAACAAGACCTCAGCTTTTCTCCCTGCGCTAAGGCCATGGGACCTGCAGAGAAATCC |
| | TGGCTCTGCTCTGGGCTTCAGTCTCTCATCTGCCCAAGAGGCTTCCTAGCCCTAGCCCAG |
| | GCTGGAGTCCCAGAGGAGCGAATGCAGTGGCATTTGGGTGAGTCAGGAGCTCTGGAGAGC |
| | TTGATGGTCACAGTGACACAAGTGACTCTGTCTCTCTGGGATTTGGTTTCTTCATCTGCC |
| | AAATGGGAATCAAGATCCTAGGCTTGTGGGGAAGGTGAAAAGGCTGAATCAGACACTGTG |
| | CACAGAGCGCCTAGCCGAGTCCTCTGCCCTGGGTACTGGCGCTCGAGGTGGACTCAGAAG |
| | CTCCAGGGCATCTGGTTCCACAAAGGACCCAGCCTGTCCCAGGCCACTGTCACCCCTGGG |
| | AGTGGCACACACTGGAGGGAATGCCTCGCTCCCAGCCCACACGTGCACACTCAGCTTCTG |
| | CCATTGCGGGCAAAATTGGACTTGACCAATTCAGGATACAAGCATAACATGTGAATATAT |
| | GCTTGCAAACACACGTGTGAGCTCACGGGCCTCACCCGCTCAGGACTCCCTCTGTGCACT |
| | CACATGCACTTGGCATTCTTGCCCATAGAGGCCCTGCTGCTGGAGAAGGAGGCTGTCTGG |
| | GGAGAGGAGGTGGAGTTTTCACAGGTTGGGCCCAGCACTGCCCCAAGAAGGAGGCTAGTG |
| | GGACGCTTGCCTCCCCAGAGCAGGTGTCATGCTGGGGATTGGGCTGTCAGTGAAGGAGGG |
| | GTGTGATGGAAGGTGAGCAAGGAAGGCTTCGGGAGAGCAAGAGGTGGGGCACCACTTGTG |
| | GGAGTCCAGGAGTGAGGGCATGTTAGTGGAGAAAGTCGGAAAGACCCAGAGGCAAGAAGG |
| | CAGGGGGTACCGAGACATATAAATGATGGCTGAATGGCGAGATGGTAATAGACGAATAGA |
| | TCACAGGTAGATGGATGCGTAGATAGAGAGATAGATGGAGAGAGAGAGAGAGAGAGAGAG |
| | AGAGAGAGAGAGAGACAAGCTGGAGAAGGTGGATAGCTAAAGCCAGAGAGACACATGGAG |
| | AGTCAGGGGACTAAAACCAGGGAGGGTGGGACCAAGAGCTTTAGAGAGAGTGAATTCCAT |
| | GGGGATCGAGTTCCAGAAATCAAAAGAGAACCAGACAGAGAGAGAAAGGAAAAAAAGAGA |
| | AACAGAGAAAACTAGACACAGAAAACCAATACGAGAAACACAGAGTGAAAGAGACCCAGA |
| | AAGAGAGAGAGAGAAGACAGGGGAGACAGGGGTCCCAGAAACAGCGACCTCAGAAACAAG |
| | GACAGATGGGGTTCTGGGCGCTCCACTGAAAGCCGGATAAGATCACCCAATGACAGGTAC |
| | CAGGAAACAGAGAGCAGGAGAGAGCCAGAGAGAGAGCAAGCGGAGACAGTCAGCCAGCCA |
| | GACACATAAATAGAAAGAGAAAGACGGACCCACAGAGAGAGAAGTAGGCCCCAGAAGAGG |
| | GAGAGACCAGCAGGCCCTCCTGAACCAGAGCAGCTCCAGGATTCTGGAATCAGACTCACT |
| | CACCCAGGCCTTCACACTCCCTGAACCCTGCAGACCCCTTCCCAGGCCTGGCTTGCCCCA |
| | CTCATCTCTGCTCCATCGTGGCCTATGGGTAGAGCTCGAAGAGAGGTGGGGAGGGGAGGT |
| | GGCCCCATGGGCAGCCGTGGGGGCTTTGATTAGCAGCTGAGAAAAGGGGCACGCTGGAAG |
| | GGTTTATCCTCAACTCAATGGCCCTGCTTCACCCCAGGCTTGGTCTCACACAGGCAGTGA |
| | TCCCAGAGCAACTTCCTGGCACAGATGGGAAAACTGAGGTCCAGATAGGGGAAGGGACTC |
| | CCCTAGTCCTCTCTCTTCAGTCTCCAGACCCCACCTGGGCCTGCTGTTTCATTTTCAAAT |
| | CACTTCTGCTCATCACCCAATACAAGAACGCTGTGGACAGAGAGCCTCTCCTCTACCTCC |
| | AGGATGGGGCCTGTGTGGGACTTCCTCCCAGCCCCCAGACTCACcgccacgaagaacagg |
| | gtgaagaggtagaccatggcctccatgtggaaccgcctcttggccgcgatgctgacagtg |
| | gggaggaaggccaggctgctgagggtgggcaggagcagcttggccaccagcgtccccatg |
| | ggccaggaggaaagcactggctggggtggggagggtgctggtgtcccaggtccccagcac |
| | aggagcacgaagtgggaaggccagctccctttgggcagggc |
| | (SEQ ID NO: 13) |
| Human | gccctgcccaaagggagctggccttcccacttcgtgctcctgtgctggggacctgggaca |
| (− strand, | ccagcaccctccccaccccagccagtgctttcctcctggcccatggggacgctggtggcc |
| reverse | aagctgctcctgcccaccctcagcagcctggccttcctccccactgtcagcatcgcggcc |
| complement)- | aagaggcggttccacatggaggccatggtctacctcttcaccctgttcttcgtggcgGTG |
| start codon | AGTCTGGGGGCTGGGAGGAAGTCCCACACAGGCCCCATCCTGGAGGTAGAGGAGAGGCTC |
| is bold & | TCTGTCCACAGCGTTCTTGTATTGGGTGATGAGCAGAAGTGATTTGAAAATGAAACAGCA |
| underlined; | GGCCCAGGTGGGGTCTGGAGACTGAAGAGAGAGGACTAGGGGAGTCCCTTCCCCTATCTG |
| stop codon | GACCTCAGTTTTCCCATCTGTGCCAGGAAGTTGCTCTGGGATCACTGCCTGTGTGAGACC |
| is bold and | AAGCCTGGGGTGAAGCAGGGCCATTGAGTTGAGGATAAACCCTTCCAGCGTGCCCCTTTT |
| italicized | CTCAGCTGCTAATCAAAGCCCCCACGGCTGCCCATGGGGCCACCTCCCCTCCCCACCTCT |
| | CTTCGAGCTCTACCCATAGGCCACGATGGAGCAGAGATGAGTGGGGCAAGCCAGGCCTGG |
| | GAAGGGGTCTGCAGGGTTCAGGGAGTGTGAAGGCCTGGGTGAGTGAGTCTGATTCCAGAA |
| | TCCTGGAGCTGCTCTGGTTCAGGAGGGCCTGCTGGTCTCTCCCTCTTCTGGGGCCTACTT |
| | CTCTCTCTGTGGGTCCGTCTTTCTCTTTCTATTTATGTGTCTGGCTGGCTGACTGTCTCC |
| | GCTTGCTCTCTCTCTGGCTCTCTCCTGCTCTCTGTTTCCTGGTACCTGTCATTGGGTGAT |
| | CTTATCCGGCTTTCAGTGGAGCGCCCAGAACCCCATCTGTCCTTGTTTCTGAGGTCGCTG |
| | TTTCTGGGACCCCTGTCTCCCCTGTCTTCTCTCTCTCTCTTTCTGGGTCTCTTTCACTCT |
| | GTGTTTCTCGTATTGGTTTTCTGTGTCTAGTTTTCTCTGTTTCTCTTTTTTTCCTTTCTC |
| | TCTCTGTCTGGTTCTCTTTTGATTTCTGGAACTCGATCCCCATGGAATTCACTCTCTCTA |
| | AAGCTCTTGGTCCCACCCTCCCTGGTTTTAGTCCCCTGACTCTCCATGTGTCTCTCTGGC |
| | TTTAGCTATCCACCTTCTCCAGCTTGTCTCTCTCTCTCTCTCTCTCTCTCTCTCTCTCTC |
| | TCTCCATCTATCTCTCTATCTACGCATCCATCTACCTGTGATCTATTCGTCTATTACCAT |
| | CTCGCCATTCAGCCATCATTTATATGTCTCGGTACCCCCTGCCTTCTTGCCTCTGGGTCT |
| | TTCCGACTTTCTCCACTAACATGCCCTCACTCCTGGACTCCCACAAGTGGTGCCCCACCT |
| | CTTGCTCTCCCGAAGCCTTCCTTGCTCACCTTCCATCACACCCCTCCTTCACTGACAGCC |
| | CAATCCCCAGCATGACACCTGCTCTGGGGAGGCAAGCGTCCCACTAGCCTCCTTCTTGGG |
| | GCAGTGCTGGGCCCAACCTGTGAAAACTCCACCTCCTCTCCCCAGACAGCCTCCTTCTCC |
| | AGCAGCAGGGCCTCTATGGGCAAGAATGCCAAGTGCATGTGAGTGCACAGAGGGAGTCCT |
| | GAGCGGGTGAGGCCCGTGAGCTCACACGTGTGTTTGCAAGCATATATTCACATGTTATGC |
| | TTGTATCCTGAATTGGTCAAGTCCAATTTTGCCCGCAATGGCAGAAGCTGAGTGTGCACG |
| | TGTGGGCTGGGAGCGAGGCATTCCCTCCAGTGTGTGCCACTCCCAGGGGTGACAGTGGCC |
| | TGGGACAGGCTGGGTCCTTTGTGGAACCAGATGCCCTGGAGCTTCTGAGTCCACCTCGAG |
| | CGCCAGTACCCAGGGCAGAGGACTCGGCTAGGCGCTCTGTGCACAGTGTCTGATTCAGCC |
| | TTTTCACCTTCCCCACAAGCCTAGGATCTTGATTCCCATTTGGCAGATGAAGAAACCAAA |
| | TCCCAGAGAGACAGAGTCACTTGTGTCACTGTGACCATCAAGCTCTCCAGAGCTCCTGAC |
| | TCACCCAAATGCCACTGCATTCGCTCCTCTGGGACTCCAGCCTGGGCTAGGGCTAGGAAG |
| | CCTCTTGGGCAGATGAGAGACTGAAGCCCAGAGCAGAGCCAGGATTTCTCTGCAGGTCCC |
| | ATGGCCTTAGCGCAGGGAGAAAAGCTGAGGTCTTGTTCTCAAGGCTCCTGTTAGACCTAG |
| | CCCAGCAAACCACAAGCACCCACCAGGCCACACCTCCCTGCCTGGGCCTAGGCTGGCCCC |
| | GCAGCTGCCTCTGCACTGGTCTCAGAGGCCCCACCCTGCCCCACCCAGAGTGGCAGGGGC |
| | AGCTGCCTCTTGTGACAAACGTCTTCTGAACTCAAAGCCACACTCAGTCCCTCCCAAAGT |
| | TCTGCCACCCGCATGGCGTCCCTTCCCTTCCAATCAGGCCCCTCCCGTGACAATCTCCAT |
| | CTCTGTGCCGGACACCTTGCTATGGGTGCAAACCCCACGGAGACAGTGTGCTTGTCCCCA |
| | CACAACAGAGGAGGCCCCCCCCCGACAGCCACTCACCTGAGCCCCCACTGGGAACATCTG |
| | CAAAGTCAGACTCCCCGAGCCTGAGCTCTGACCCCCCAGGCCCTCAGCCGCACCCACCCC |
| | ACCAGTGCCCCGGCTGGGCCAGGTCTGCCCCCTGCACTGCAGGATGGGAGGAAGTGGCCA |
| | GGTGCCAAGCACTGCTCTGTTTCACCTCTTTGGGCAGGCAGTGCTGCTCTGAAGTTCACT |
| | TTCCCCATCTGTCGAATGGAGCTAAGTTCCCACCATTGGGAGGAGCCAAGGGCCTAGTGG |
| | CAGAGGCTCTGGGTCCCCAGGGTGAGCTGGCTGTCCTTTAGAATTCTCAGAGCCTGGCGT |
| | CCCCCTCCCTGAACACATGCAGCAAGCACTCTATGCTGGGAGCCGTCCATGCAACTGGGA |
| | CCAGACAGAGCCATTCCTGCTCCTGGCAGGGCAAGGGCACAGGCCTTATCACAATCTCAC |
| | AGTGAATGCCTTAATCACACATGACCATCTGTGCTACATTGAGGCACACAGACAGCAGGT |
| | GATGGGGCTGGGGTGGGGGATGGGAAGAAGCCAGGAGAACAGGAGAACAGCCTAGGGCCG |
| | GAGTCTTCCTCGCTCCTCTTTCCCACAGAGGTGTAAGCACCTTGATCAGGCCCCTCCTGA |
| | AGCTTCCCTGTCCCCAAGGTAAACAGTCATGTCCACCGCTGGTCACCCTCCTCTAGCCGC |
| | GTCTTTCCCAGTCTCACTTCAAGAAAGGTGCACCACACAGAGCTCGGCATAGTCTTCTGG |
| | ATGAGCCTGACAAGAACAGAGAGAAGCAGGACCGCCGCCTCCCTCATTTTAGATGCTGGA |
| | TCCCTATTAATGCCTCCTAGGTGCACCGTCACTTTAGGCCGTGTACAATTGCCATCAGTA |
| | GAAGCACTCCACTTTGACCCCGAATTGCCTGCCCCCCAGATCACCCAGGGCCCCCGCTCC |
| | CTCTGTGCTGTCCCTGTCGCCTCCAGACACGGGCTGCTTTCCAGCACGCCTGCTCCTCCC |
| | GCTGAGCTGGGCTGACCAAGAGTCCACCGTGCTGTTTCCCAAGCTTGTTTGTGCCAGTTG |
| | AACCTGTCAAAATAATTATGTCATGTAATTAAATATTACCTAAAATGATGAAATGCGAAT |
| | GCAAAAGGAGAGAGAGTTGTTTTCCGTAGAAAAGTAGTTGACTGCTTTGAAAACACTCCC |
| | TATAAGCCTTTTGCCTTAAAAATTGCCATGAAAGTGGGTGTTGGGGAGGCGACGACGAGA |
| | GTATAGGGCAGAGATGGATGAGCCTAGGGGGCTGTGTTCAGCTTCTGCAAGGGGGTCTCA |
| | GAGATCTTCGTCCACGTTAAGGCAACAGAAACCAGGAGCTACAGCCCGGCTCTCAGTGTG |
| | GGCCCTGCCAGCCTCAGCAGTGCCACAGGGAATGTGTTCAAAATGCACATTTTCTACCCA |
| | ACCCAGCCCTCCCTAATCAGCAACCCTAGGCAGGGCTCAGTAACCTGTGTCTAAACAAGG |
| | CCCCAGGCGTTCTGATGCCCACTCAAGCCTGAGAACGCTGCTGCAGTGTAGACCAGTCAT |
| | CAGAGGGGTAATTTGTGCAAGAGAAGGCCCCAGGAGCCCCCAAAGAGCAAAGGAGCCCCC |
| | AGAGAGGCTCTGACCTCACATCCAAAGATTGGCGAGTGAACACGCAGCTGTGTGCTTCAA |
| | GGAAGCACAGATGCTGAAGGTCCGGGGAGAGCCGTGTAGGGTCCCTCCCGGAGACCGCCC |
| | CACCTAAGCGGGCTTCACCTGAGTGACCACTCAGTCTCCGCCCAGTCCTGCCAGTGCAGA |
| | TGAGCGAATTCTGCTGAGGTGACATTGAGTGAACCCTTGGCACACCCCAGCCCCTAAACA |
| | CCTCGCTGGCATGTGCCACTTAATCCCCACAGGACCCTCTGCGGGAGTGCTCGGCAGTAC |
| | CCCCATTTTTCAGACGGGGAGACTGAGGCACACAGTCATTAAGTGACTCGCCAAAGTGAC |
| | TTCTCACAGCCAGCAAGTGACCTGGCACTCTGGGGGCCGTGGCTCGGGGACCAGCTCTGG |
| | CCTCTTTGTGCTGTGACCACCTGTGGGTTTTCTGGCAGctccaccatgcctgcaatggac |
| | ccggcttgtctgtgctgtgcttcatgcgtcacgacatcctggagtatttcagtgtctacg |
| | ggacagccctgagcatgtgggtctcgctgatggGTGAGTGGTCAACCCCTGCAGACAAGC |
| | CTTGCGGACAAGGACCGGCTCCCCAGTGTCCGCACACCCAGCCTCAAACCAGTCCCGCTG |
| | CAGCCCTCTATCCCCACTCACTCCCAGCCCCAGGAATGTCTTCCCTTGTCTGGGTTTCAG |
| | GGTTGTGCTTAATCTATGAAAAAGAATATGCACCTCTTAAATGCCCTGATGCCGATTTTT |
| | TATATCCTTTTAAAATGACTCATTTAGTAAAAGCTTGTTGTGACACATTTCAACAGCAGA |
| | GGCATACAAAGGAAAAAGTGAAAGGGCTCCCTCCCGTCACTCCTTCCCTGGGAGGACCGC |
| | TGTTCACTGCTCAGGGGGGTTGGCTTCCCAGACTGTCATACAAACATCCACACACACGGG |
| | TGATATCCAGCCTGGAAGACGGGACTGTTCATAAGCCCCCTTTCCCACCATGTTTTTTTC |
| | CACGTAACTGTTGTCTGTGGCAAGGTCCCCATGACCTGAGGCTCCTTGCCATCTAACCGG |
| | GCCTGTGAGGGGCTTCATTTCTTCTCTTGAATCTTAGTGTCTTTGTCCTGTGATCCTATG |
| | GGATAGATTCGATCCTATTCACTTTGCATAGTGATTATTGATTTTTAGCCACAGGGTCTC |
| | ACTCTGTCACTCAGGCCAGAGTACAGTGGCACAAACACGGCTCACTGCAGCCTCGAACTC |
| | ACAGCCTCAAGCCATCCTCCCACCTCAGCCTCCCAAGTAGCTGGGATGACAGGCATGCAC |
| | CACGACACCCACCGAATTTTTTGTGGAGATGGGGGTCCCGCTATGTTGCCCAGGCTAGTC |
| | TTGAACTCCTGGTCTCAGACGATCCTCCTGCCTCTGCCTCCCAAAGCACTGGGATTGCAG |
| | ACCTGAGCCACCACAACCAGCTGTGGTCTGTTTATTAAAATGAGGTGCTGGGAGCACAAT |
| | CCAAGCTGCTGTGTCCATGGGGAAGCAGCAGGCTGCCTGTCCTCTCAAACTGGCTGGAAT |
| | TCAGACCCCTCCATGACCAGGCTGGGCCTCAGGGTCTCGCTGGCTGAGGGGACACCGAGA |
| | GAGGGCATCTACAGGATGCCTCCTGGGGGCTGGGGGGTGGGGGGGTAGCCAGGAGGAGCA |
| | AGAGAGACCACTCACCTCCCCCTGTGTCTCCCTGGCCCCTCCAGcactggccgacttcga |
| | cgaacccaagaggtcaacatttgtgatgttcggcgtcctgaccattgctgtgcggatcta |
| | ccatgaccgatggggctacggggtgtactcgggccccatcggcacagccatcctcatcat |
| | cgcggcaaagtggGTGCGTACTGCGCGAGCCTCGATGAACGGGGACCCAGGAGGCCCGTC |
| | ACCTCTCCCCTGTCTGACTCCTCCTGCCTCGGATCTTCATTTACCTCCTGCCCTCATAGG |
| | CAAATGGGTGGATGCAGGAATTCAGCAAAACCACGTAGAAAATAGCCATGAGGTGCTCCG |
| | GCTGTGTAGACTGTGTGTGACGCTTGTCTGTACTCACCAGCTCCAGACTGAGCCCAAGTT |
| | ACCCAATGTCTCACCTCCACGCCCGTACCTGTAAAGCAGGTGTTGTCATTACAGCCCCCT |
| | GCCTGTCTCATGGGGTGGTTCAAAGACTCACTGAGATCAGGCACCTGAAGTCCTGGCATG |
| | GAGCCTGGCTTCCAATGTGCTCCGCACATAAGAGCTATTTGTTATCACTCTCATCACCGC |
| | TCTTACTACCAGCTGCTTTAGAGATGCAACATCAACTTGGAAAATGGCCTGGGGTAATCT |
| | GGGAAGACTTCCTGGAGGGAGTGAGCCTCGAGCTTGGTTCTAGCCAGGAGGGAGAGTGTG |
| | GTATGACAGTGAGGCAGCCTGGGCCAGACCTTCTAGCACAGACAGCCCCTTGCAGAGCAG |
| | CAAGGCCCTCACGTGGTCTTCAGTGCTCCCTGCTCTCTCCGTCCTACCAAGACCCTCCTG |
| | GCTCAGGGACCAAACCCCACTCAAACTCGCCCAAGCTAAAGAGGGATCTTAGGGAAAAAT |
| | AGACCCGGGGCATCTCCTGGGACCCCAGAGCAGGAGGGAGCTCACCCTTGCAAGGGTCTC |
| | CCAGACCTGGGGAGCCAGCAGGCTTCAGCCTGAGCCTGCTCTCCAGGCCTGCAGCAGCCC |
| | AGGCACCGCCTGAACATCCCTGTGCTGCATTTCACCTCTTTGGAGTGGACCTGAAGTCTC |
| | AGGGTCAGTTTCAAGTGCCAAGAGAGGACCTGGCCTGGTCCAGCCCAGCCACACTGCCAG |
| | CCCCGGCCAGAGCCGCAGGACACCCAGCAAGACAGAGGGCAGCTCTCAGTGCAGGAGGGC |
| | CCACGGCTGTGGGGAGAGCGCTCGAGTTCTGGTGGGCAGCTTGCAAAGCCCGTCCCAATC |
| | CTCCCCACAGCCAAGGAGCATGGGACTGATTTGCCATTTGGCCTATGAAGGAACCAAAGC |
| | ATGGAGGCCTCAGCAACTTAACAGGACGTCATGGCAGGGCAGTGGCGGGGCCAGAACCTG |
| | ACTTTTTTTTTTTTTTTTTTGAGACGGAGTTTTGCTCTTGTCACCCAGGCTGGAGTGCAA |
| | TGGCGCCATCTTGGCTCACTGCAACCTCCGCCTCCCGGGTTGAAGCGATTCTCCTGTCTC |
| | AGCCTCCCGAGTAGCTGGGATTATAGGCGCATGCCATCACGCCCAGGTAATTTTTATATT |
| | TTTAGTAGCGATGGGGTTTCACCACGTTGGCCCGGCTGGTCCTGAGCTCCTGACTTCAGG |
| | TAATCTGCCTGCCTCAGTCTCCCAAAGTGCTGGAATTACAGGCATGAGCCACCACGCCTG |
| | GCTGGACTTGACTTTTTGAGCAGGAGACAGAGGACCTGCTTTCTGTCTGCAACCTGAGGA |
| | AGCAGCACTAATCCTGGGCTGACCCAGGAGGCCCCTGGGAGTCACCTGGTGGGGAGAGAG |
| | GGAGCGCTGGGCATAAGTGGGCTGGACCAAGGTGGGTGGCAGGAAGGAGCGTGCTGGGGC |
| | CCTGGGTCCTGGCCACCCGCCTCTCGCCTCCTTCTGCCCCTGAAGGTTGCTCCTCCCGCT |
| | GGTCTTGGCCAGGTCTGAACTGGGCTGAACTGTAATTGCCCAGAAGAGAATTCACTGCTG |
| | TGTTTCCCACCAAGAGCCACGAGACTGCCAAGGCCCTCAGGCTGCAGATCCAGCCACAGG |
| | AGCCGAGGATGGGCTGCCCGCCTCCCCGCATCTGGGGCTCAAATTGTTGTCTTCTAGACC |
| | ATTTAGTATGGAGTTTATTTAGGATTTTCAAGGGCAATTGTTTCCTGGAATGAGGGTGGA |
| | TTTTTCTCCCTGAGCCTCGTCCCCTCTTGGGAGGGGTTGGGCAATGGCAGCCCGGGAGGA |
| | AGAAGGAGGGAGGGTTGGGTGATGGCGCCCTTTCAATAGTGCCAGGCCCACTGTGTGACC |
| | TGGGGCAGGCACCTCTCTTCTCCCTGGTGTTACTACTGCAGGGCTGGCCAGAGGTGGGCT |
| | GGCAGCCTTGACCACCGCACCCAGGGTCACACGGTCCAGCTGTCTTCAGGCCACTGCAGG |
| | ACTGCAGGTGCTCAATTCCTTGTTGGCAATGGAATTCCAGAAAATGGAAAGCCGGGCATC |
| | AATTCATAATTCACTGGGTGGCAAAACCTGACCTGACCCAGCATAAGGCTCTTTGTGCTC |
| | TTTGCATAGGCCACTCAGCAATGGGTGCAGTTATGGGGTGCTGCCCCCACCCCCAGGGGC |
| | TTGTGCCACACGCTGTGTGTCTAACAGTTGATTCCACTGAAATCTATAGCCTCCTGAGTG |
| | TACCCCCACGGGTCTGGCCCCACTCCGAGGCCGCCATCACCCAACCCTCCCTCCTTCCTC |
| | CCCAACAAGGCTGTCGTTCCCCAGCCCCTCCCAAGAGGGGACCAGGCTCAGGGAGAAAAA |
| | TCCACCCTCATTCCAGGAAACAACTGCCCTTGAAAATCCCAAATAGACTCCATGTGAAGT |
| | GGCCTAGAAAACACAAGGCCGGCTCCAGGGATGTGGCCCCACACTTCCCGCATCTTCCTC |
| | GGAATGCAAACCCCTCTGTAAGTGGAAACGCCCACGCGAGCCTTCCTAAGCACTGGGACC |
| | TGAGAAGCAGTGCTGGCCAAGTTTGTCTAATGAAGAGTGAGATAGGTCCTTTAACTAGGA |
| | AAGCAAACGATGCTTGTCCATGGCTCAGGGATCACCACAAGTGCCATCCCCATGGGCCAC |
| | ACTGGGCTGGCAGGTCCCAAGTACTCCAGTACTTTCCTTGGTAAGCAATCATGGAGTCTA |
| | GTCCCCCCAGAGCTCCCCTCCAGGGAGGAGGAGACATGCGAGGGTGGCGTTGTTACATCT |
| | CAAAGAGGCGGGAGGGCAGCGGGGTCTTTGTGGGGCACATGGCCCAGTGGCTTCCTTCCC |
| | AGCCATCCAGGCTGGGGTCTCTGCTGGGTCGCACCGTGAAGGGCCCTGTCCAGCAGAAGC |
| | CATCTGAGGCTGGGTGCGGCAGGAGGCTCCTGAGCCTCCTCTCCTGCCGGGAGAGGGCTG |
| | GGGTGAGCCTGTGCAGGGCCAGCTCTCGGGCTGGTTTGGGGAGGGGCGTTCCCCAGTGCT |
| | GTGACCTAAAAGCTTTGCTGTGGCCTCCTGTCCTCACAGctacagaagatgaaggagaag |
| | aagggcctgtacccagacaagagcgtctacacccagcagataggccccggcctctgcttc |
| | ggggcgctggccctgatgctacgcttcttctttgagGTACCAAGCCCAGCGGGGAGGTGT |
| | CCTGTGCCCCAGAGCCCACTCGGCATGGCTCTGCTCAGCTGGGATACCAGGGCTGTGCCC |
| | ACGCTGGGGGGACTCTGACAGCCCCAGGCCAAAGTCTGGTATTAGTCCCTGCTCACTAGC |
| | CCCTCCCAGGGCTATGTCGGGGGCAATGCCGTCCCCAGCACCACCAGCACCATGGCCTGT |
| | GTGGACCTGGAGGTCACAGAGACCAAAGCTGGCTGCCTACAGACAGCTGGCATCTCAGAC |
| | GGCAGCACAGCCTGGGAGACGCCTGGGGCAGGTGCCCAACACCTGGAGGAGGGAGGGAGG |
| | GAGGGGAGGAGACAGGGAAGGAAGGAGGGAGAAAGTGGAGGAGGGAGGGAGGAACAGGGG |
| | AAGAGGGATAAGGAGAGAAAAGAGGGGGAGATGGAGGGAGGGAGGGTAGGAGGGAGAGAA |
| | GGATGGAGGAAGAGAAGGACAGCAGAGAGAGGGGATGGGGAAGAAGGAAGGAGTGCAGCC |
| | TGGAGGATGGCACAGGCCCTCACTGCTCTGGGTTTAGGTGGAGAGAAAAACCACTGTGAG |
| | CGCGACCGTGGGGGAGGCTTCCTGCAGGTGTCCCAGTGCCCTGGCAGAGGCGGGGGCCTG |
| | AAGGGGCTCTGCTGGGTCTTGGAGAGGGAGGAGCCTCGGGGAGGGGGCTGAGGCCCCAGA |
| | CTGACCGCTTGCCCCCCGCAGgactgggactacacttatgtccacagcttctaccactgt |
| | gccctggctatgtcctttgttctgctgctgcccaaggtcaacaagaaggctggatccccg |
| | gggaccccggccaagctggactgctccaccctgtgctgtgcttgtgtc [symbol] tgctgcgcc |
| | cagcccggctctgagcccctgccctccccagctcacacttg |
| | (SEQ ID NO: 14) |
| Mouse | ttccaggaactagaatgtatgttaggcgaagctaatgactagtggctgatcaagagttta |
| (+ strand) | ctgtgaatggcttgatcgaaaacctgcagaagggatgggactcaggcaggggtatgcaag |
| | gttcgctggctccagcttcctaagtggagagctttcagagcctgggcaggggttaaaagg |
| | gcaatcccagtttcctagggaaagcagacgattctgacaggcaggacctgggaaatagat |
| | aaccctgcatgctgctgggtatttactggtctagggttctctgccaggcacacctatggt |
| | tgtgaggccttgggggataaagttcttttttttcctgaacagagtgaagcaactggtgaa |
| | cacagaaccagtgggtccctaagcagcactcagcagaatgcagcaggcctgctggtctct |
| | tggggtgtagagaagaccatttctcatgtacaggccgcataacaaagtataggaagtacc |
| | ttgggagagacagcaggactgccaggcaggaaggcaggggcctggtgtgtgtgtgtgtgt |
| | gggggggtatagtcagacacaagtgcagcagagggtggagaaggtcagcttggcgggggc |
| | ccctgcgttcccagccttcttgttgaccttgggcagcagcaggacaaaggacatggccag |
| | ggcacagtggtagaagctgtggacgtaggtgtaatcccattcCTGTGGAGGAGAATGAGT |
| | CAGTCTGGGCCTCCATCCCTTCCCTAAACCAAGTCCTAGCCATTTGGTGCCTCTGTCAGC |
| | CAGCCCACCCTGAGAAGGTGGCAGAAAGGCTTGCTGCCTTCCTCTGTTCCATGCCTCCTG |
| | GGTGCTGGGCACCAGCTCCTGGTTCCTTCCAGGACATGCGTGCATCTTGGGTGCAGGCTT |
| | CCTAAAGTCAGGGCCTGACTTGTCCACTCAGGCAGTGAGGCTAGTACACTGGGGATGGTG |
| | AGTACCATCCTCAAGAGGACAGAATTTACAACTTGGAGCCTCCATATGTGGCTGTTAGTT |
| | AACTATTTCCAGAGGCTCTTGCTCCCCTTCCCCATAGGCCAGGTACctcaaagaagaatc |
| | gaagcatcagggccagggccccaaagcacaggccggggcctatctgctgggtgtagatgc |
| | tcttgtcggggtacaggcccttcttctctttcatcttcttcagCTGCAGGCACAAGGTGG |
| | GGACATCAAAGTTCTTGGGGTGCAGCACAGGAAGGGACCCCTCCATGAACTGTAGAAGAG |
| | CCCTACCCCCATTCCTCTGTATGCCTGACTGATGGGACTCTCTGGGCCAATTTCCCCTGG |
| | GTCCTCTACTGCCCGCATCTGGTGGGCTTTGGCACTTCAGTGGCAGACGTGATCAGTTTT |
| | CCCAGCTAAGGGGTTTTCCTCTGTTAACCTTGGTTTCATAGGCCCTGTGTGTTCAAGCTT |
| | GGTAAGATGGAGTGTTACATGGAATAGATGGGAGTCCCATGGTTCCTCACTGGAATGCAC |
| | ATCCTTGGGGCCCAAAGGTATTTTAGGTATTCAAGATTGTTCAGGTTTCAGTGGGGAAGA |
| | TCATTATAAATACCACTGTCAGGTGTGCACAGAGGGCACAGGACAGCAGCCCTGACTGAG |
| | TGATGTGCACAGTGGGCACAGGACAGCGGCCCTAATTGCACACCTCACTAAATACATTAT |
| | ATGTACAAATGCTGTCAATGGCCTCGTGCAAATCAGGGCAAGCTTTGTCACTCTGAGTGA |
| | TGATATGTTGCTGTTTCCAAGTGTTCTAAAACTTGCCATTAGTAACAGGAGTGGAGGTCC |
| | CAGTGAGCAGTGCCAGTGACATGGGCACCGCCTATTAGCCTGAGTGTAGGCCGTATGACC |
| | ATCAATCACACAGTTCTAACACTGGGGCCCCAGAGAGGAGAAGAATATTGAAGATCACCC |
| | ATGGGCCCTGTCTTGCCCCGGGAACCCCTATTTCCCATTTCACTCAGCTTCTTCTCCCCA |
| | AATGTTGTATTCATGTTCCTTTCCTGAAAGGGTGAGACATGGGAAAGAATTGTACTCCGT |
| | TCTAAGAAGTAAGTCCAAACCACCTGCCTATCTAAGATCTAGGAGATGGGGTCTGTGCCC |
| | CAGGCATGGGTGGCTGCAGCCCCTCACTCCCATTCTCACCAGAGACCTGGGGAGGCTGGC |
| | ATTTAGTGGAGGGGGGCACTGGCACATGTATGCTATCCTGGCTAATTAAAATCCCATCAG |
| | GATGGGTGTGCTGGGCTTGGACACCAGCATTCAAGAGGCAGAGGCGGGCAGATCTCTATG |
| | AGTTTGAAGCCATCCAGAGATACAAAGTGAGAGTCTATCTTTAAAAACAAACAAACAAAC |
| | AAACAAACAAACAAACAATCAAGTCAGATCCAGAACCAGTGAAGAGCAGCAAGGGGCCAT |
| | GATAGGCAAGACAAAGAGGCAGTTATCAGAGCAAGCCTTCTTGTTTATGCATTCCAGCTT |
| | GTTAACTAGCCATGCAGAAGCCCAACACCTCTGCCTTGGGTCAGAGAGGGCCAGCTTCGG |
| | CTCCTCAAACTGGAGTGGGATGGAAGCTTCTCCCCTCGAAAGTCAAGCACAGCTGCCATT |
| | ACCTACTAGGGCTGCAGGTTAGGCTGCTGAGCTCTGTGCATTTCAGGTTCATCCTTAACT |
| | TAAAATCAGAATAAGCCCGGGTTCCTCGGAGCCCACAGGAGTAGGATGTGGCTTGGAAGC |
| | TTCCTCCCTGACTATACCTGTCCCCACTTTGCTGAAGATGGATCAGAGCTCTCCCACCCC |
| | TGGCCCTGCCACTCCCCTCTGACACAGACACAGACACAGACACAGACACAGACACAGACA |
| | CAGACACAGACACAGACACAGACACAGACACAGACACAGACACAGACAGACACAGACACA |
| | GACAGACACAGACACAGACACAGACACAGACACAGACACAGACACAGACACAGACACAGA |
| | CACAGACACAGACACAGACACACAGGCATAGACACAGACACAGACAGACACAGACACAGA |
| | CACAGACACAGACACAGACACAGACACAGACACAGACACAGACACAGACACAGACACAGA |
| | CACAGACGACACAGACACAGACAGACACAGACACAGACAGACACAGACACAGACAGACAC |
| | AGACACAGACACAGACACAGACGACACAGACACAGACAGACACAGACACAGACACAGACA |
| | CAGACGACACAGACACAGACAGACACAGACACAGACACAGACACAGACACAGACACAGAC |
| | ACAGACACAGACACAGACACAGATGACACAGACACAGACGACACAGACACAGTCACAGAC |
| | ACAGACACAGACGACACAGACACAGACAGACACAGACACAGACAGACACAGACACAGACG |
| | ACACAGACACAGACACAGACACAGACGACACAGACACAGACAGACACAGACACAGACACA |
| | GACAGACACAGACACAGACAGACACAGACACAGACACAGACACAGACACAGACACAGACG |
| | ACACAGACACAGACAGACACAGACACAGACACAGACACAGACATAGACACAGACACAGAG |
| | ACACAGACACAGACAACACAGACACAGACACAGACACAGACACAGACACAGACTCAGACA |
| | CAGACACAGACACAGACACAGACACAGACACAGACTCAGACTCAGACTCAGACTCAGACT |
| | CAGACTCAGACTCAGACTCAGACTCAGACACACAGTCACACAGACACACACAGACACACA |
| | CAGACACACACACAAAGGCACACACACACACAAAGGCACACACACACACACACACCCCAC |
| | CGCCTGCCCCAATCTGCACTGCTGTAGCTCTACTTCCAGGAACCTGCAAGATCCCAAATG |
| | GTGCTTCCTGCATGAGGTAGCAGACAGGTGAGAACTTGAAGCCTGAGTGCTGTCTGCTTG |
| | GTCTGAAGCCTGCTGGCCTGGAAGGTCTGTGTTTTGGGCCTAACTGCTCTGGGTGGAGCT |
| | CAGAAAACATCCCTGGGTCTTTCCTGCTATTGGGAAATTTGTTCACGATGGCTAACTTAG |
| | GTGGGTTTTAGGCCATGGAGGTGAGAGGGCCTTGAGACCATAGAGGGGTTAGGAGCCTAT |
| | ACAGCAGAGTAGATGCCAAGCGCCAGGCCCTCCTCTAGGCCTCCCACTCAATACCCTGCT |
| | TCACCCCCACCTCACACCTTCCTTCCTCATGAGAACCATTTCCAAGGCTTGCTTCTTTCG |
| | GGAAGACTATCCAGATTAACCTATCTGCTCTCCAAACTGGTATTATACCTGTAAGCAGTG |
| | TTGTCTCTCAGAATGATAATGATAGTGATCTTATGTTGATGAAAAGACTGACAGTGACAG |
| | TCATGATGACAAAAGGTCTCCTCAGCTCTGGGTATATTAAAAATCACACCTGTGCCTGTG |
| | CCTGTGCTTAGAAGCATTCTTTATGGGTATTTGGATGCAGAGCAGAGGTCAAGAGAAAAG |
| | GAGTTTTGGCTTTATCCAGGACCAATAAACCAGCAGGGCATGGGACCCGAGCATGAGCCA |
| | CCATTTTTAGGAATTTTAGGGTTTTTGGCCCAATTCTTACTAATTCACCTGCATATAAGG |
| | ATATGGGGTATAGGACCCTACATAGGAGAAACCAAGATCAGGGAAGAAATGCAGGTTCCG |
| | TGGTCTCCGACAGTGGAGATACTGGAAGTACTCACccactttacagcaatgatgagggtg |
| | gccgtgcctatgggaccggagtataccccgtaaccccagcggtcatgaaaagtccgcaca |
| | gcgatggtaaggacgccaagcattgtgaaggtcgatctctggggttcatcaaagtcggcc |
| | agtgCTGGGGAGAGGCATAGCTATGGTGAGCAGCGTCCCTCACATGGCTGTGCCTCCATC |
| | CTTGGGAACCTATTGGTATGTCCTCTCAATCTGTAGGGCCAGCCTGGTTTCCATAAGGTC |
| | TGAATTTTGGTTATTTGGAGGGAGTGGGTGATGCTGCTTCCCTGGAGCAGGGTGGCTGAA |
| | ATAAACTGGTAGACTGAGTGACCAGCATTTCCTAGGAATCCTGAGACAAAAGTGTTAAGA |
| | CTAATGATTGGTGCGCAGAGCTGAGTCTCAGGAGGGACCCCGGGACTGCATCCCTGGGAG |
| | ACAAGGGTGAGCTTGCTTGGTTTCTCCCTTTTCTCTTTCCTTCCCTTCCCTTTCTTTCCC |
| | CTTTCTTTGCTCTCTCCCTCCTTCTCTCCCTGTCTCCCTTCCTCCCTCCCTCCCTGTCTC |
| | CTTTCCTTTCTTCTTCCTTCATCCTTTCTGCTTTCTACTTTTCTCTATCTCTTCCTTCTT |
| | TTTCTTTCAAAATTTTGCAGTTGCTGGTAATGGAATGAAGGGCCTATTGATTACCAGGCG |
| | AGCGATCTGCCATTGAGCTGTATATACCCCAGGTCCAAGGTGAGGATTTTGAATGGTCTG |
| | CCTTCCTAATACACAGAGCTGAGCTGACCCATGAGGGCAAATGCTCCTCTGAGCCTGGAG |
| | GACAAGCTGGGAGGCTAGGTCCAGGATGCCTTTGGCCTCTCCTTTGTATGCTTCTGTTTT |
| | TTAAATGTCACAAGTGCTAACTACTGGAGTCACTTAAGGATGGTGGAAATGAGAGTGCAG |
| | GCATCAGAGAAATGTGCATGTCTCTTTAAGCAGATTAAGCTCTGCAAAGCAGCAAGGAGG |
| | GAGGATCTCAGAGAGGGGCTGGGTACTGGCTGGGGTTCAGGACTGGCTCCCACCCATTGG |
| | CCAAGATGGCCACTTACccatcagggagacccacatgctcagggctgttccatagatgct |
| | gaagtactccagaatgtcacggcgcatgaagcacagcacagacaaaccaggcccatcaca |
| | ggcatgggagaaCTGTAGGGAAATCACATGAGGTCAGCAGGCAGTGGGCAGCCCAGGAGT |
| | GGGTGAGAACTGGTCCCAAGGCTCAGGTTCACTAGCTGTGAGCCCCTAATGGTTTTGTAC |
| | CTCAGCCTCCTCCCTCACACTATCAGAGCCCTTGTGGAGATTAAACAGGTGAGTCCATCT |
| | AGCCTGGGAGTGCAAAAGTCTTTGTAAATATCCCTTTCAGACTCAGCACTGGCCCAAGGC |
| | TGGTGAGAAGCATGCTCAGAAGGGCATCCTTAAAGACCACTTACacctttgcccatgact |
| | gactgaaagtgtacacattcctatgccagtctttgcataggagccttttatcctggaccc |
| | ctgtctctccataaaagaggaagcccttagattccccccaagcaagtgctgatTCTGACA |
| | CACTGGTTTCTTTCCCCCATATGCCAGCAGGTGTGTCCCTGACTCGTAGTTGAATAGATT |
| | TGCTTCTAAGCAAAAGGTTCTATATGCAGGATTTCCAAGCAGACAACTTATTTCTTGCAG |
| | AAAACAACTTGCTCTCCCTTTGCTTCACATTTCATCATTTTAAGTAATATTTAATTACAT |
| | GACATAATTATTTTGACAAGTGCAACTGGCACAAACAAGCCCAGCAGCCAGCACAATGAG |
| | CTCTTGGTAAGCCCAACTTAGCAGGAGGGAGCAGGCAAGCTGGAAAACAGCCTTGTCTGG |
| | AGGCAGCAGGGGCACCACCGAGGGAGGCAGGCGGAGAGCTGGGGACCCTGGATGATGGAT |
| | GTATCAGTCAAGCACATAGGGCCTACTTAGAAGCTCAGAGACCTCCTGCTGGTCACAGTT |
| | GCACATGGACTCTGTCAATCAATAGAGAGCATCCAGGGGAAGGGAGGAGGTGGTCCAGCC |
| | TCTGTGTTGGGTCAGCCCCAGCCTTGAGCTTTGGGTTCTGCACCTTTTAAAAGGGAGATT |
| | GGTGAAGAGGAGGTTAACCAACTAGGTATGAGCTCAGGAAAAGACAAGCTTTGGGTTGGG |
| | CCAGACCAAGGTACGCAAGTGGAGAAGGAAAGGAACTCAGCTCTGGGAGGGGACTCTGCT |
| | CTTCTGGCTCCTTACAGAACCACATGACCCCACCCCACCCCACCTGAGCCCATCATCTGT |
| | AGCATCTTGCTTCCTTCTCTTGTATAGGCCCCCATGAATGAGTAAAACTTACTTACTGTG |
| | AGATCCTGGGAAACACTCATGCCTTCCCCCACGAGGAGAAGAGCTTCCTTAGGCTTGATC |
| | TCAACATAGAATACTTGGCTACATGTGAAGGCCAGAGGAGCAGGCTTTCTAACAAGGGAT |
| | CTAACTGTCCTCAGGCCCTGAGGATTAATTTTTTGGGGGGTGGGTGACCTGTGTGACAGT |
| | GAACTTCCCTGGGGAACCTCCTGCCCAAGGAGGCAGGGGCAAGGCTGTGATGTGTACCCT |
| | TTCTCCCCAGAGGCAGGGAGATCTGGTCCAGCTGGTGCCAGGCTAGGACACAGCTGGGTG |
| | TGACAGGAGCCCTAACCCTGCTGTCAGCTCAGAGCTGGCAGAGGGGCCCAGGTTCTCTCA |
| | GGTCTCTCAGGCCCACCTTGTCTAATGGCATGAGAACACCTGTTCTGTGGGGCTTACAAG |
| | GGGACCCTAACGATAACTGCGGAGCATGGCACCCCACACTGCAAAAATGAAATGCTGTTT |
| | AAAGTTTGCTTTCATTAATCAAACTTTCCCCCAACCTGAAACCAAGTTAATATGTGCGTT |
| | ATGGGCATTTAAACAATGTGCTTGCCCTGGGCAGAATTAGCTCACCTCTGGGAAAAACAA |
| | TTCAATCGATCTTATTATGCTTTGCATTTCTGGTGGAGGACTCTAGTGAGTCTTTGTGAC |
| | TCTTTCATGCCCGACTCAGAACAGTATATGTTTGTGTGAGATGTGGTGACCAGGTCTAAG |
| | ACCACGTGTGTTAGAAACAGCAAGGTATGGAGACCATGTTGAAAGCAAAATGTGGGTGTA |
| | GGCTGATAATATCTGATTGTGGATTTGTGTGCTACTGAGTCAAAGGGCCAGAGAGACAGC |
| | TGTCTGCTATAAAAGCCTAAGACTCAGATCCCATTCTTTTTGTCCCTGTTTGTTGTGCTG |
| | TTCAGCAAGTAGAAAGGATGATATTGTCTAAGATTCTTAGATTAGAACCTGATTTTAGAT |
| | TAGATGACTATCAGGTTAGAACAGGAGAGGGCAGAATTCTTTGGAATACATCAGATCCAC |
| | CCGCTGTGTAACTGACACCAAGAGTCATTCTTCTATTCAGCAGCAGCATACCATACAACT |
| | GGTAGTTGTCATGGAGAGTCCTACAGCAGCCACGTGGAAGGCAGAACTCTGTGAGGAACA |
| | GATTGTGGCTTTGAGGCCAGAGGACATTTGTCATAAGAGACAGCTGGCCCTGCCACTCTG |
| | GGTGGGGTGTGGCAGGGTGGGCCTCCAAGGCCAGTGCAGAGGCAGCTGTAGGCCAATTAG |
| | ACCCAGGCAGGCAGGGGTGACCTGATTGGGGCTGTGATTTGCTGGACTGTATCTAACACA |
| | GGCCTTGGGAACAAGACCCTGGCTTATGTCCTTGACCGTGGGGTCTCATCTTGGCTCTGA |
| | CCTTGGCCAGGTCTCAAGAGGAACAAATGACAGTGTGGGACAAAGTACTGTGGGGCAGAC |
| | CAGGATCTGAGTGTTCATGGTGACACTGGTGGCCCAGTTTCTCTGAGACTCAGTTTCCTC |
| | TTCTATCAAATTGAAATCACTATGTTAGGCTCGTGGGTGATAATGAGTCCAACCCCACCA |
| | TGGTTGCTTTCTTGTGACTTATCATTGGCCTAATGTCCTCCCCTACTGAAGTGAACTCAA |
| | GAGCCATAGAGTTTCCAGTTCCTTGGGTTACCTATGGGACCACCACAACCAGGAGGTAGA |
| | CAGGTGCCAAGCCCTCCCCCACTGTTCTCAGCCCACATGCATTGTGGCTTCTCCCACCAC |
| | TAGAAAGTCATGCCAGCTGACTCAGGATATGGAACACGCATGTGAGCACAGATGTGTGAG |
| | TTTGTGGGCTCACTCATTGAGAGCCAGCTGGATACCTTCACATACTCTATGCCCTTGCCT |
| | TACTGAGACCTGCTGCAGGAAGGGCAGGCCTAAGGAGAGGATGCTAGTCTCTAAAAGTTT |
| | GGCTCTGCTCTAAGGAGGAGACTAGCAGGCTGCTTGCCAACCCTGAGCATGTATCCTACC |
| | AGTGTGTGGGCCTCACACCAGACAAACTAGTGAGGCATAGTGTGATGAGAGAGAAACGAA |
| | GGTTACAGAGTGGTAAAAGAGACAGTGTGACTCCTGGTTAGAGGATAGCTGAGAGGGCCA |
| | TCATGAGAGGTACTCAGAAGGACTAAAGGGCAAAGTGAGAGGAGGCCTTTAAGACAGAGA |
| | GTAGATGGGTAGATGAATGGACAGGGAGAGAGATGGTTGGTTAGCAGATAATAGAGAAAT |
| | GATAGACAGATAGACAGACAGACAGATGATGGATAGACACATAGAACAAGACAAATGATA |
| | AATGAATAGATGATAGACAAAGGAGATAGAGAGACAGAAGCAAGTTGAATGGGCAGGAAG |
| | ATAAAGTCAGGAAGACACAGAGCTCTGGTCAAGAACCCAGGGGAGAGCAGACCAGGGAGA |
| | AGAGGAGAGTGAACTCCTCGGGGGAGTGTAACTCTAGAAATCAGAAAAAAACAAAAAAAA |
| | AAAACCCCAAAAAACAAACAAACAAACAAACAAAAAAATTGGATACAGGCAGGAAGAAGG |
| | AAGAGATAGAGACTGGGAGAAACTAGACACAGAACCAGTCAAAGAAGCAGAGGGAGAGAG |
| | ACCCATGGCGGGAATAAAGAGAAGCAGAAACCCAGACACAAGGCTTCAGCAAAGCTGGGC |
| | CAGTGCCAGACATGCCCCGAACGAACGACAGAGGAGTCACCCAGTACTGTTGCCTGGGAA |
| | CAGAGTGGAGAAGGAACTAAGAGGCAGCCAGCCAGCTAGACACATAACAGGAAGAGAAAG |
| | AAGGACTCAGGGAGAGGCTGGCTCCTCTCAGTGGGGGTAGTTCCAAATTCTGGAGCTGCA |
| | GTCACCCAGGCCCTCTACCTTTCCTGAACCTAGTAGATCCATTCCTAGGCCTGCTCACTC |
| | ACCTTGTTCCTCCTCAGCTGAGCAACTCATGGAACAACGTTGGTAGAAAGGAGAGAGAGT |
| | CTGAGGAGCACCAGGCTTGACCTTAACTGACACCGGGCTCTCATGGGCCTGGCCTCAGTC |
| | TCAGGTGTCAATCACCCCCCTCAAATGTCTGGCGCACATGGAGAAACTGAGGTCCACAGA |
| | GGAAGACAGATTCCAGGAACCTTCTCTTCCCAGTCACCACCCCCACTGCTCCCCCAGACC |
| | CAGACTCTTTCTCTTCCAAATCCTGTTTCTGCATCACCTGGCACAGGACAATGGTGGTAA |
| | CCCTCCCGTGAGGACTTCCTCCTAATTTCCTCCTTCCACACTTACcgccacaaagaacat |
| | ggtgaagaggtagaccatggcctccatgtagaaacgcctcttggtagcgatgctcactgt |
| | cgggaggaaggccaggctgctgagggtaggcaggagcagtttggctacaactgtccccat |
| | ggaccaggaggaaggcactgactggggagaaggtggtaaaggcccccctggtctccaggg |
| | caggaagaaaaagagcccacttctttgcttctccagcagccctgaccgcagctgtggcag |
| | cacccacaaggagggcttaagtgctc (SEQ ID NO: 15) |
| Mouse | gagcacttaagccctccttgtgggtgctgccacagctgcggtcagggctgctggagaagc |
| (− strand, | aaagaagtgggctctttttcttcctgccctggagaccaggggggcctttaccaccttctc |
| reverse | cccagtcagtgccttcctcctggtccatggggacagttgtagccaaactgctcctgccta |
| complement)- | ccctcagcagcctggccttcctcccgacagtgagcatcgctaccaagaggcgtttctaca |
| start codon | tggaggccatggtctacctcttcaccatgttctttgtggcgGTAAGTGTGGAAGGAGGAA |
| is bold & | ATTAGGAGGAAGTCCTCACGGGAGGGTTACCACCATTGTCCTGTGCCAGGTGATGCAGAA |
| underlined; | ACAGGATTTGGAAGAGAAAGAGTCTGGGTCTGGGGGAGCAGTGGGGGTGGTGACTGGGAA |
| stop codon | GAGAAGGTTCCTGGAATCTGTCTTCCTCTGTGGACCTCAGTTTCTCCATGTGCGCCAGAC |
| is bold and | ATTTGAGGGGGGTGATTGACACCTGAGACTGAGGCCAGGCCCATGAGAGCCCGGTGTCAG |
| italicized | TTAAGGTCAAGCCTGGTGCTCCTCAGACTCTCTCTCCTTTCTACCAACGTTGTTCCATGA |
| | GTTGCTCAGCTGAGGAGGAACAAGGTGAGTGAGCAGGCCTAGGAATGGATCTACTAGGTT |
| | CAGGAAAGGTAGAGGGCCTGGGTGACTGCAGCTCCAGAATTTGGAACTACCCCCACTGAG |
| | AGGAGCCAGCCTCTCCCTGAGTCCTTCTTTCTCTTCCTGTTATGTGTCTAGCTGGCTGGC |
| | TGCCTCTTAGTTCCTTCTCCACTCTGTTCCCAGGCAACAGTACTGGGTGACTCCTCTGTC |
| | GTTCGTTCGGGGCATGTCTGGCACTGGCCCAGCTTTGCTGAAGCCTTGTGTCTGGGTTTC |
| | TGCTTCTCTTTATTCCCGCCATGGGTCTCTCTCCCTCTGCTTCTTTGACTGGTTCTGTGT |
| | CTAGTTTCTCCCAGTCTCTATCTCTTCCTTCTTCCTGCCTGTATCCAATTTTTTTGTTTG |
| | TTTGTTTGTTTGTTTTTTGGGGTTTTTTTTTTTTGTTTTTTTCTGATTTCTAGAGTTACA |
| | CTCCCCCGAGGAGTTCACTCTCCTCTTCTCCCTGGTCTGCTCTCCCCTGGGTTCTTGACC |
| | AGAGCTCTGTGTCTTCCTGACTTTATCTTCCTGCCCATTCAACTTGCTTCTGTCTCTCTA |
| | TCTCCTTTGTCTATCATCTATTCATTTATCATTTGTCTTGTTCTATGTGTCTATCCATCA |
| | TCTGTCTGTCTGTCTATCTGTCTATCATTTCTCTATTATCTGCTAACCAACCATCTCTCT |
| | CCCTGTCCATTCATCTACCCATCTACTCTCTGTCTTAAAGGCCTCCTCTCACTTTGCCCT |
| | TTAGTCCTTCTGAGTACCTCTCATGATGGCCCTCTCAGCTATCCTCTAACCAGGAGTCAC |
| | ACTGTCTCTTTTACCACTCTGTAACCTTCGTTTCTCTCTCATCACACTATGCCTCACTAG |
| | TTTGTCTGGTGTGAGGCCCACACACTGGTAGGATACATGCTCAGGGTTGGCAAGCAGCCT |
| | GCTAGTCTCCTCCTTAGAGCAGAGCCAAACTTTTAGAGACTAGCATCCTCTCCTTAGGCC |
| | TGCCCTTCCTGCAGCAGGTCTCAGTAAGGCAAGGGCATAGAGTATGTGAAGGTATCCAGC |
| | TGGCTCTCAATGAGTGAGCCCACAAACTCACACATCTGTGCTCACATGCGTGTTCCATAT |
| | CCTGAGTCAGCTGGCATGACTTTCTAGTGGTGGGAGAAGCCACAATGCATGTGGGCTGAG |
| | AACAGTGGGGGAGGGCTTGGCACCTGTCTACCTCCTGGTTGTGGTGGTCCCATAGGTAAC |
| | CCAAGGAACTGGAAACTCTATGGCTCTTGAGTTCACTTCAGTAGGGGAGGACATTAGGCC |
| | AATGATAAGTCACAAGAAAGCAACCATGGTGGGGTTGGACTCATTATCACCCACGAGCCT |
| | AACATAGTGATTTCAATTTGATAGAAGAGGAAACTGAGTCTCAGAGAAACTGGGCCACCA |
| | GTGTCACCATGAACACTCAGATCCTGGTCTGCCCCACAGTACTTTGTCCCACACTGTCAT |
| | TTGTTCCTCTTGAGACCTGGCCAAGGTCAGAGCCAAGATGAGACCCCACGGTCAAGGACA |
| | TAAGCCAGGGTCTTGTTCCCAAGGCCTGTGTTAGATACAGTCCAGCAAATCACAGCCCCA |
| | ATCAGGTCACCCCTGCCTGCCTGGGTCTAATTGGCCTACAGCTGCCTCTGCACTGGCCTT |
| | GGAGGCCCACCCTGCCACACCCCACCCAGAGTGGCAGGGCCAGCTGTCTCTTATGACAAA |
| | TGTCCTCTGGCCTCAAAGCCACAATCTGTTCCTCACAGAGTTCTGCCTTCCACGTGGCTG |
| | CTGTAGGACTCTCCATGACAACTACCAGTTGTATGGTATGCTGCTGCTGAATAGAAGAAT |
| | GACTCTTGGTGTCAGTTACACAGCGGGTGGATCTGATGTATTCCAAAGAATTCTGCCCTC |
| | TCCTGTTCTAACCTGATAGTCATCTAATCTAAAATCAGGTTCTAATCTAAGAATCTTAGA |
| | CAATATCATCCTTTCTACTTGCTGAACAGCACAACAAACAGGGACAAAAAGAATGGGATC |
| | TGAGTCTTAGGCTTTTATAGCAGACAGCTGTCTCTCTGGCCCTTTGACTCAGTAGCACAC |
| | AAATCCACAATCAGATATTATCAGCCTACACCCACATTTTGCTTTCAACATGGTCTCCAT |
| | ACCTTGCTGTTTCTAACACACGTGGTCTTAGACCTGGTCACCACATCTCACACAAACATA |
| | TACTGTTCTGAGTCGGGCATGAAAGAGTCACAAAGACTCACTAGAGTCCTCCACCAGAAA |
| | TGCAAAGCATAATAAGATCGATTGAATTGTTTTTCCCAGAGGTGAGCTAATTCTGCCCAG |
| | GGCAAGCACATTGTTTAAATGCCCATAACGCACATATTAACTTGGTTTCAGGTTGGGGGA |
| | AAGTTTGATTAATGAAAGCAAACTTTAAACAGCATTTCATTTTTGCAGTGTGGGGTGCCA |
| | TGCTCCGCAGTTATCGTTAGGGTCCCCTTGTAAGCCCCACAGAACAGGTGTTCTCATGCC |
| | ATTAGACAAGGTGGGCCTGAGAGACCTGAGAGAACCTGGGCCCCTCTGCCAGCTCTGAGC |
| | TGACAGCAGGGTTAGGGCTCCTGTCACACCCAGCTGTGTCCTAGCCTGGCACCAGCTGGA |
| | CCAGATCTCCCTGCCTCTGGGGAGAAAGGGTACACATCACAGCCTTGCCCCTGCCTCCTT |
| | GGGCAGGAGGTTCCCCAGGGAAGTTCACTGTCACACAGGTCACCCACCCCCCAAAAAATT |
| | AATCCTCAGGGCCTGAGGACAGTTAGATCCCTTGTTAGAAAGCCTGCTCCTCTGGCCTTC |
| | ACATGTAGCCAAGTATTCTATGTTGAGATCAAGCCTAAGGAAGCTCTTCTCCTCGTGGGG |
| | GAAGGCATGAGTGTTTCCCAGGATCTCACAGTAAGTAAGTTTTACTCATTCATGGGGGCC |
| | TATACAAGAGAAGGAAGCAAGATGCTACAGATGATGGGCTCAGGTGGGGTGGGGTGGGGT |
| | CATGTGGTTCTGTAAGGAGCCAGAAGAGCAGAGTCCCCTCCCAGAGCTGAGTTCCTTTCC |
| | TTCTCCACTTGCGTACCTTGGTCTGGCCCAACCCAAAGCTTGTCTTTTCCTGAGCTCATA |
| | CCTAGTTGGTTAACCTCCTCTTCACCAATCTCCCTTTTAAAAGGTGCAGAACCCAAAGCT |
| | CAAGGCTGGGGCTGACCCAACACAGAGGCTGGACCACCTCCTCCCTTCCCCTGGATGCTC |
| | TCTATTGATTGACAGAGTCCATGTGCAACTGTGACCAGCAGGAGGTCTCTGAGCTTCTAA |
| | GTAGGCCCTATGTGCTTGACTGATACATCCATCATCCAGGGTCCCCAGCTCTCCGCCTGC |
| | CTCCCTCGGTGGTGCCCCTGCTGCCTCCAGACAAGGCTGTTTTCCAGCTTGCCTGCTCCC |
| | TCCTGCTAAGTTGGGCTTACCAAGAGCTCATTGTGCTGGCTGCTGGGCTTGTTTGTGCCA |
| | GTTGCACTTGTCAAAATAATTATGTCATGTAATTAAATATTACTTAAAATGATGAAATGT |
| | GAAGCAAAGGGAGAGCAAGTTGTTTTCTGCAAGAAATAAGTTGTCTGCTTGGAAATCCTG |
| | CATATAGAACCTTTTGCTTAGAAGCAAATCTATTCAACTACGAGTCAGGGACACACCTGC |
| | TGGCATATGGGGGAAAGAAACCAGTGTGTCAGAatcagcacttgcttggggggaatctaa |
| | gggcttcctcttttatggagagacaggggtccaggataaaaggctcctatgcaaagactg |
| | gcataggaatgtgtacactttcagtcagtcatgggcaaaggtGTAAGTGGTCTTTAAGGA |
| | TGCCCTTCTGAGCATGCTTCTCACCAGCCTTGGGCCAGTGCTGAGTCTGAAAGGGATATT |
| | TACAAAGACTTTTGCACTCCCAGGCTAGATGGACTCACCTGTTTAATCTCCACAAGGGCT |
| | CTGATAGTGTGAGGGAGGAGGCTGAGGTACAAAACCATTAGGGGCTCACAGCTAGTGAAC |
| | CTGAGCCTTGGGACCAGTTCTCACCCACTCCTGGGCTGCCCACTGCCTGCTGACCTCATG |
| | TGATTTCCCTACAGttctcccatgcctgtgatgggcctggtttgtctgtgctgtgcttca |
| | tgcgccgtgacattctggagtacttcagcatctatggaacagccctgagcatgtgggtct |
| | ccctgatggGTAAGTGGCCATCTTGGCCAATGGGTGGGAGCCAGTCCTGAACCCCAGCCA |
| | GTACCCAGCCCCTCTCTGAGATCCTCCCTCCTTGCTGCTTTGCAGAGCTTAATCTGCTTA |
| | AAGAGACATGCACATTTCTCTGATGCCTGCACTCTCATTTCCACCATCCTTAAGTGACTC |
| | CAGTAGTTAGCACTTGTGACATTTAAAAAACAGAAGCATACAAAGGAGAGGCCAAAGGCA |
| | TCCTGGACCTAGCCTCCCAGCTTGTCCTCCAGGCTCAGAGGAGCATTTGCCCTCATGGGT |
| | CAGCTCAGCTCTGTGTATTAGGAAGGCAGACCATTCAAAATCCTCACCTTGGACCTGGGG |
| | TATATACAGCTCAATGGCAGATCGCTCGCCTGGTAATCAATAGGCCCTTCATTCCATTAC |
| | CAGCAACTGCAAAATTTTGAAAGAAAAAGAAGGAAGAGATAGAGAAAAGTAGAAAGCAGA |
| | AAGGATGAAGGAAGAAGAAAGGAAAGGAGACAGGGAGGGAGGGAGGAAGGGAGACAGGGA |
| | GAGAAGGAGGGAGAGAGCAAAGAAAGGGGAAAGAAAGGGAAGGGAAGGAAAGAGAAAAGG |
| | GAGAAACCAAGCAAGCTCACCCTTGTCTCCCAGGGATGCAGTCCCGGGGTCCCTCCTGAG |
| | ACTCAGCTCTGCGCACCAATCATTAGTCTTAACACTTTTGTCTCAGGATTCCTAGGAAAT |
| | GCTGGTCACTCAGTCTACCAGTTTATTTCAGCCACCCTGCTCCAGGGAAGCAGCATCACC |
| | CACTCCCTCCAAATAACCAAAATTCAGACCTTATGGAAACCAGGCTGGCCCTACAGATTG |
| | AGAGGACATACCAATAGGTTCCCAAGGATGGAGGCACAGCCATGTGAGGGACGCTGCTCA |
| | CCATAGCTATGCCTCTCCCCAGcactggccgactttgatgaaccccagagatcgaccttc |
| | acaatgcttggcgtccttaccatcgctgtgcggacttttcatgaccgctggggttacggg |
| | gtatactccggtcccataggcacggccaccctcatcattgctgtaaagtggGTGAGTACT |
| | TCCAGTATCTCCACTGTCGGAGACCACGGAACCTGCATTTCTTCCCTGATCTTGGTTTCT |
| | CCTATGTAGGGTCCTATACCCCATATCCTTATATGCAGGTGAATTAGTAAGAATTGGGCC |
| | AAAAACCCTAAAATTCCTAAAAATGGTGGCTCATGCTCGGGTCCCATGCCCTGCTGGTTT |
| | ATTGGTCCTGGATAAAGCCAAAACTCCTTTTCTCTTGACCTCTGCTCTGCATCCAAATAC |
| | CCATAAAGAATGCTTCTAAGCACAGGCACAGGCACAGGTGTGATTTTTAATATACCCAGA |
| | GCTGAGGAGACCTTTTGTCATCATGACTGTCACTGTCAGTCTTTTCATCAACATAAGATC |
| | ACTATCATTATCATTCTGAGAGACAACACTGCTTACAGGTATAATACCAGTTTGGAGAGC |
| | AGATAGGTTAATCTGGATAGTCTTCCCGAAAGAAGCAAGCCTTGGAAATGGTTCTCATGA |
| | GGAAGGAAGGTGTGAGGTGGGGGTGAAGCAGGGTATTGAGTGGGAGGCCTAGAGGAGGGC |
| | CTGGCGCTTGGCATCTACTCTGCTGTATAGGCTCCTAACCCCTCTATGGTCTCAAGGCCC |
| | TCTCACCTCCATGGCCTAAAACCCACCTAAGTTAGCCATCGTGAACAAATTTCCCAATAG |
| | CAGGAAAGACCCAGGGATGTTTTCTGAGCTCCACCCAGAGCAGTTAGGCCCAAAACACAG |
| | ACCTTCCAGGCCAGCAGGCTTCAGACCAAGCAGACAGCACTCAGGCTTCAAGTTCTCACC |
| | TGTCTGCTACCTCATGCAGGAAGCACCATTTGGGATCTTGCAGGTTCCTGGAAGTAGAGC |
| | TACAGCAGTGCAGATTGGGGCAGGCGGTGGGGTGTGTGTGTGTGTGTGTGCCTTTGTGTG |
| | TGTGTGTGCCTTTGTGTGTGTGTCTGTGTGTGTCTGTGTGTGTCTGTGTGACTGTGTGTC |
| | TGAGTCTGAGTCTGAGTCTGAGTCTGAGTCTGAGTCTGAGTCTGAGTCTGAGTCTGTGTC |
| | TGTGTCTGTGTCTGTGTCTGTGTCTGTGTCTGAGTCTGTGTCTGTGTCTGTGTCTGTGTC |
| | TGTGTCTGTGTTGTCTGTGTCTGTGTCTCTGTGTCTGTGTCTATGTCTGTGTCTGTGTCT |
| | GTGTCTGTGTCTGTCTGTGTCTGTGTCGTCTGTGTCTGTGTCTGTGTCTGTGTCTGTGTC |
| | TGTGTCTGTCTGTGTCTGTGTCTGTCTGTGTCTGTGTCTGTGTCTGTCTGTGTCTGTGTC |
| | GTCTGTGTCTGTGTCTGTGTCTGTGTCGTCTGTGTCTGTGTCTGTCTGTGTCTGTGTCTG |
| | TCTGTGTCTGTGTCGTCTGTGTCTGTGTCTGTGACTGTGTCTGTGTCGTCTGTGTCTGTG |
| | TCATCTGTGTCTGTGTCTGTGTCTGTGTCTGTGTCTGTGTCTGTGTCTGTGTCTGTGTCT |
| | GTGTCTGTCTGTGTCTGTGTCGTCTGTGTCTGTGTCTGTGTCTGTGTCTGTCTGTGTCTG |
| | TGTCGTCTGTGTCTGTGTCTGTGTCTGTGTCTGTCTGTGTCTGTGTCTGTCTGTGTCTGT |
| | GTCTGTCTGTGTCTGTGTCGTCTGTGTCTGTGTCTGTGTCTGTGTCTGTGTCTGTGTCTG |
| | TGTCTGTGTCTGTGTCTGTGTCTGTGTCTGTGTCTGTGTCTGTCTGTGTCTGTGTCTATG |
| | CCTGTGTGTCTGTGTCTGTGTCTGTGTCTGTGTCTGTGTCTGTGTCTGTGTCTGTGTCTG |
| | TGTCTGTGTCTGTGTCTGTGTCTGTCTGTGTCTGTGTCTGTCTGTGTCTGTGTCTGTGTC |
| | TGTGTCTGTGTCTGTGTCTGTGTCTGTGTCTGTGTCTGTGTCTGTGTCTGTGTCTGTGTC |
| | TGTGTCAGAGGGGAGTGGCAGGGCCAGGGGTGGGAGAGCTCTGATCCATCTTCAGCAAAG |
| | TGGGGACAGGTATAGTCAGGGAGGAAGCTTCCAAGCCACATCCTACTCCTGTGGGCTCCG |
| | AGGAACCCGGGCTTATTCTGATTTTAAGTTAAGGATGAACCTGAAATGCACAGAGCTCAG |
| | CAGCCTAACCTGCAGCCCTAGTAGGTAATGGCAGCTGTGCTTGACTTTCGAGGGGAGAAG |
| | CTTCCATCCCACTCCAGTTTGAGGAGCCGAAGCTGGCCCTCTCTGACCCAAGGCAGAGGT |
| | GTTGGGCTTCTGCATGGCTAGTTAACAAGCTGGAATGCATAAACAAGAAGGCTTGCTCTG |
| | ATAACTGCCTCTTTGTCTTGCCTATCATGGCCCCTTGCTGCTCTTCACTGGTTCTGGATC |
| | TGACTTGATTGTTTGTTTGTTTGTTTGTTTGTTTGTTTGTTTTTAAAGATAGACTCTCAC |
| | TTTGTATCTCTGGATGGCTTCAAACTCATAGAGATCTGCCCGCCTCTGCCTCTTGAATGC |
| | TGGTGTCCAAGCCCAGCACACCCATCCTGATGGGATTTTAATTAGCCAGGATAGCATACA |
| | TGTGCCAGTGCCCCCCTCCACTAAATGCCAGCCTCCCCAGGTCTCTGGTGAGAATGGGAG |
| | TGAGGGGCTGCAGCCACCCATGCCTGGGGCACAGACCCCATCTCCTAGATCTTAGATAGG |
| | CAGGTGGTTTGGACTTACTTCTTAGAACGGAGTACAATTCTTTCCCATGTCTCACCCTTT |
| | CAGGAAAGGAACATGAATACAACATTTGGGGAGAAGAAGCTGAGTGAAATGGGAAATAGG |
| | GGTTCCCGGGGCAAGACAGGGCCCATGGGTGATCTTCAATATTCTTCTCCTCTCTGGGGC |
| | CCCAGTGTTAGAACTGTGTGATTGATGGTCATACGGCCTACACTCAGGCTAATAGGCGGT |
| | GCCCATGTCACTGGCACTGCTCACTGGGACCTCCACTCCTGTTACTAATGGCAAGTTTTA |
| | GAACACTTGGAAACAGCAACATATCATCACTCAGAGTGACAAAGCTTGCCCTGATTTGCA |
| | CGAGGCCATTGACAGCATTTGTACATATAATGTATTTAGTGAGGTGTGCAATTAGGGCCG |
| | CTGTCCTGTGCCCACTGTGCACATCACTCAGTCAGGGCTGCTGTCCTGTGCCCTCTGTGC |
| | ACACCTGACAGTGGTATTTATAATGATCTTCCCCACTGAAACCTGAACAATCTTGAATAC |
| | CTAAAATACCTTTGGGCCCCAAGGATGTGCATTCCAGTGAGGAACCATGGGACTCCCATC |
| | TATTCCATGTAACACTCCATCTTACCAAGCTTGAACACACAGGGCCTATGAAACCAAGGT |
| | TAACAGAGGAAAACCCCTTAGCTGGGAAAACTGATCACGTCTGCCACTGAAGTGCCAAAG |
| | CCCACCAGATGCGGGCAGTAGAGGACCCAGGGGAAATTGGCCCAGAGAGTCCCATCAGTC |
| | AGGCATACAGAGGAATGGGGGTAGGGCTCTTCTACAGTTCATGGAGGGGTCCCTTCCTGT |
| | GCTGCACCCCAAGAACTTTGATGTCCCCACCTTGTGCCTGCAGctgaagaagatgaaaga |
| | gaagaagggcctgtaccccgacaagagcatctacacccagcagataggccccggcctgtg |
| | ctttggggccctggccctgatgcttcgattcttctttgagGTACCTGGCCTATGGGGAAG |
| | GGGAGCAAGAGCCTCTGGAAATAGTTAACTAACAGCCACATATGGAGGCTCCAAGTTGTA |
| | AATTCTGTCCTCTTGAGGATGGTACTCACCATCCCCAGTGTACTAGCCTCACTGCCTGAG |
| | TGGACAAGTCAGGCCCTGACTTTAGGAAGCCTGCACCCAAGATGCACGCATGTCCTGGAA |
| | GGAACCAGGAGCTGGTGCCCAGCACCCAGGAGGCATGGAACAGAGGAAGGCAGCAAGCCT |
| | TTCTGCCACCTTCTCAGGGTGGGCTGGCTGACAGAGGCACCAAATGGCTAGGACTTGGTT |
| | TAGGGAAGGGATGGAGGCCCAGACTGACTCATTCTCCTCCACAGgaatgggattacacct |
| | acgtccacagcttctaccactgtgccctggccatgtcctttgtcctgctgctgcccaagg |
| | tcaacaagaaggctgggaacgcaggggcccccgccaagctgaccttctccaccctctgct |
| | gcacttgtgtctgactatacccccccacacacacacacacaccaggcccctgccttcctg |
| | cctggcagtcctgctgtctctcccaaggtacttcctatactttgttatgcggcctgtaca |
| | tgagaaatggtcttctctacaccccaagagaccagcaggcctgctgcattctgctgagtg |
| | ctgcttagggacccactggttctgtgttcaccagttgcttcactctgttcaggaaaaaaa |
| | agaactttatcccccaaggcctcacaaccataggtgtgcctggcagagaaccctagacca |
| | gtaaatacccagcagcatgcagggttatctatttcccaggtcctgcctgtcagaatcgtc |
| | tgctttccctaggaaactgggattgcccttttaacccctgcccaggctctgaaagctctc |
| | cacttaggaagctggagccagcgaaccttgcatacccctgcctgagtcccatcccttctg |
| | caggttttcgatcaagccattcacagtaaactcttgatcagccactagtcattagcttcg |
| | cctaacatacattctagttcctggaa (SEQ ID NO: 16) |

One or more modifications, in some instances, can include an insertion, a deletion, a substitution, or combinations thereof. In some embodiments, the inventive polypeptide does not encompass one or more naturally occurring polypeptides (e.g., does not encompass one or more of the wt-myomaker polypeptides). In other embodiments, the inventive polypeptide does not encompass any of the wt-myomaker polypeptides. In some embodiments, the inventive polypeptide does not encompass any naturally occurring polypeptide (e.g., does not encompass any of the wt-myomaker polypeptides or any other naturally occurring polypeptide).

In some embodiments, one or more modifications to a wt-myomaker polypeptide can include one or more substitutions, one or more insertions, or one or more deletions (or combinations thereof) to one or more amino acids in a hydrophobic region of a wt-myomaker polypeptide, to one or more amino acids in a hydrophilic region of a wt-myomaker polypeptide, or in a combination thereof. In some embodiments, one or more modifications to a wt-myomaker polypeptide can include one or more substitutions or one or more deletions (or combinations thereof) to one or more amino acids in a hydrophobic region of a wt-myomaker polypeptide, to one or more amino acids in a hydrophilic region of a wt-myomaker polypeptide, or in a combination thereof.

In some embodiments, the myomaker polypeptide can have a polypeptide sequence with an amino acid sequence identity to a wt-myomaker polypeptide (e.g., SEQ ID NO:1 or SEQ ID NO:4) of about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 70%, at least about 80%, at least about 90%, at least about 95%, at least about 99%, or at least about 99.5%. In some embodiments, the myomaker polypeptide sequence has an amino acid sequence identity to SEQ ID NO:1 or SEQ ID NO:4 of about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 70%, at least about 80%, at least about 90%, at least about 95%, at least about 99%, or at least about 99.5%. The amino acid sequence identity (e.g., percent identity) can be determined by any suitable method, such as using BLAST, BLAST-2, ALIGN, ALIGN-2, Clustal Omega, or Megalign software. Unless otherwise indicated, the amino acid sequence identity (e.g., percent identity) is determined using BLAST-2.

Nucleic acid molecules that encode for the myomaker polypeptide are termed “myomaker nucleic acid molecules.” In certain embodiments, the myomaker nucleic acid molecule is included in a vector (e.g., a viral vector, a retroviral vector, a lentiviral vector, an adenoviral vector, an adeno-associated viral vector, a herpesviral vector, a chimeric viral vector, a plasmid, an expression vector, a conjugative vector, or a nonconjugative vector). In certain embodiments, the myomaker nucleic acid molecule is in a cell, such as an insect cell (e.g., an Sf9 cell) or mammalian cell (e.g., a human cell, a rat cell a mouse cell, a muscle cell, a non-muscle cell, a myoblast, a fibroblast, a C2C12 cell, a 10T ½ fibroblast, a NIH/3T3 cell, a CHO cell, a mesenchymal stem cell (MSC), a hematopoietic stem cell, a blood cell, a bone marrow cell, or an adipose stem cell).

In other embodiments, the myomaker nucleic acid molecule comprises one or more nucleic acid sequences that are not used to encode for the myomaker polypeptide (e.g., one or more introns). For example, the myomaker nucleic acid molecule can include one or more nucleic acid molecules as found in nature (e.g., including introns). In certain embodiments, the myomaker nucleic acid molecule differs from the one or more nucleic acid molecules in nature because the myomaker nucleic acid molecule does not include one or more introns. In some embodiments, the myomaker nucleic acid molecule is a cDNA molecule (“myomaker cDNA molecule”). In certain embodiments, the myomaker cDNA molecule is identical to a nucleic acid molecule found in nature. In other embodiments, the myomaker cDNA molecule is not identical to a nucleic acid molecule found in nature (e.g., due to the myomaker cDNA molecule not including one or more introns in the nucleic acid molecule found in nature).

In some embodiments, the myomaker nucleic acid molecule sequence has a sequence identity to a nucleic acid molecule encoding a wt-myomaker polypeptide (e.g., SEQ ID NO:7, SEQ ID NO:10, SEQ ID NO:13, SEQ ID NO:14, SEQ ID NO:15, or SEQ ID NO:16) of about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 90%, at least about 95%, at least about 99%, or at least about 99.5%. In some embodiments, the myomaker nucleic acid molecule sequence has a sequence identity to SEQ ID NO:7, SEQ ID NO:10, SEQ ID NO:13, SEQ ID NO:14, SEQ ID NO:15, or SEQ ID NO:16 of about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.95%, about 99.99%, less than about 100%, at least about 90%, at least about 95%, at least about 99%, or at least about 99.5%. Nonlimiting examples of wt-myomaker polypeptides and wt-myomaker nucleic acid molecules can be found in Table 2. The nucleic acid sequence identity (e.g., percent identity) can be determined by any suitable method, such as using BLAST, BLAST-2, ALIGN, ALIGN-2, Clustal Omega, CRISPor Megalign software. Unless otherwise indicated, the nucleic acid sequence identity (e.g., percent identity) is determined using BLAST-2.

In some embodiments, the myomaker nucleic acid molecule encodes for a myomaker polypeptide that has one or more modifications to wt-myomaker polypeptide in a hydrophobic region, in a hydrophilic region, or in a combination thereof.

The myomaker nucleic acid molecule can be made using any suitable technique, such as but not limited to, those found in WO 2014/210448 A1, those found in WO 2018/152103 A1, chemical synthesis, enzymatic production or biological production. Chemical synthesis of a nucleic acid molecule can include, for example, a nucleic acid molecule made by in vitro chemical synthesis using phosphotriester, phosphite or phosphoramidite chemistry and solid phase techniques, or via deoxynucleoside H-phosphonate intermediates. Enzymatically produced nucleic acid molecules can be accomplished using any suitable method including but not limited to Polymerase Chain Reaction (PCR). Biologically produced nucleic acid molecules can be accomplished using any suitable method including but not limited to a recombinant nucleic acid produced (i.e., replicated) in a living cell, such as a recombinant DNA vector replicated in bacteria.

Modifications or changes made in the structure of the myomaker nucleic acid molecules and/or myomaker polypeptides can be used in the present invention. In certain embodiments, a myomaker polypeptide can be modified (e.g., by one or more insertions, one or more deletions, or one or more substitutions (e.g., conservative substitutions)). In some embodiments, the myomaker polypeptide which was modified does not have an appreciable loss (e.g., a decrease in a function of less than about 1%, less than about 5%, less than about 10%, less than about 25%, less than about 50%, less than about 75%, less than about 90%, less than about 95%, less than about 99%, or less than about 100%) of one or more functions of the unmodified myomaker polypeptide such as, for example, the ability to activate fusion of two cells, the ability to make a cell fusion capable (e.g., a protein confers fusion capable properties to a cell if upon adding the protein, the cell is capable of fusing to another cell if that other cell comprises myomaker and myomerger), the ability to confer fusogenicity to a cell (e.g., a protein confers fusogenic properties to a cell if upon adding the protein, the cell will fuse with another cell if that other cell comprises myomaker), the level of expression during embryonic development, the level of expression during myogenesis in adult organisms (e.g., older than embryonic), the level of induction of myogenesis in adult organisms (e.g., older than embryonic), the affinity for membranes, or the level of association with membrane compartment. In some embodiments, the myomaker polypeptide which was modified retains desired levels (e.g., at least about 20%, at least about 40%, at least about 50%, at least about 75%, at least about 80%, at least about 90%, at least about 95%, or at least about 99%) of one or more functions of the unmodified myomaker polypeptide, such as, for example, the ability to activate fusion of two cells, the ability to make a cell fusion capable (e.g., a protein confers fusion capable properties to a cell if upon adding the protein, the cell is capable of fusing to another cell if that other cell comprises myomaker and myomerger), the ability to confer fusogenicity to a cell (e.g., a protein confers fusogenic properties to a cell if upon adding the protein, the cell will fuse with another cell if that other cell comprises myomaker), the level of expression during embryonic development, the level of expression during myogenesis in adult organisms (e.g., older than embryonic), the level of induction of myogenesis in adult organisms (e.g., older than embryonic), the affinity for membranes, or the level of association with membrane compartment. In some embodiments, the myomaker polypeptide after modification has an increased level of one or more functions as compared to the unmodified myomaker polypeptide. Nucleic acid molecules can be designed to encode for such a modified myomaker polypeptide, and such nucleic acid molecules can be used in the present invention.

A “functional myomaker polypeptide” is defined as a myomaker polypeptide (e.g., a modified polypeptide) that has desired levels (e.g., at least about 20%, at least about 40%, at least about 50%, at least about 75%, at least about 80%, at least about 90%, at least about 95%, or at least about 99%, as compared to another myomaker polypeptide, such as a naturally occurring myomaker polypeptide) of one or more functions such as, for example, the ability to activate fusion of two cells, the ability to make a cell fusion capable (e.g., a protein confers fusion capable properties to a cell if upon adding the protein, the cell is capable of fusing to another cell if that other cell comprises myomaker and myomerger), the ability to confer fusogenicity to a cell (e.g., a protein confers fusogenic properties to a cell if upon adding the protein, the cell will fuse with another cell if that other cell comprises myomaker), the level of expression during embryonic development, the level of expression during myogenesis in adult organisms (e.g., older than embryonic), the level of induction of myogenesis in adult organisms (e.g., older than embryonic), the affinity for membranes, or the level of association with membrane compartment. In some embodiments, the function myomaker polypeptide has an increased level of one or more functions as compared to another myomaker polypeptide (e.g., a naturally occurring myomaker polypeptide). Nucleic acid molecules can be designed to encode for functional myomaker polypeptides, and such nucleic acid molecules can be used in the present invention.

A “functionally equivalent myomaker polypeptide” is defined as a myomaker polypeptide that has been modified (e.g., by one or more insertions, one or more deletions, or one or more substitutions (e.g., conservative substitutions)) from an original myomaker polypeptide and that modified myomaker polypeptide retains desired levels (e.g., at least about 20%, at least about 40%, at least about 50%, at least about 75%, at least about 80%, at least about 90%, at least about 95%, or at least about 99%) of one or more functions of the original myomaker polypeptide, such as, for example, the ability to activate fusion of two cells, the ability to make a cell fusion capable (e.g., a protein confers fusion capable properties to a cell if upon adding the protein, the cell is capable of fusing to another cell if that other cell comprises myomaker and myomerger), the ability to confer fusogenicity to a cell (e.g., a protein confers fusogenic properties to a cell if upon adding the protein, the cell will fuse with another cell if that other cell comprises myomaker), the level of expression during embryonic development, the level of expression during myogenesis in adult organisms (e.g., older than embryonic), the level of induction of myogenesis in adult organisms (e.g., older than embryonic), the affinity for membranes, or the level of association with membrane compartment. In some embodiments, the functionally equivalent myomaker polypeptide can have an increased level of one or more functions compared to the original myomaker polypeptide. Nucleic acid molecules can be designed to encode for functionally equivalent myomaker polypeptides, and such nucleic acid molecules can be used in the present invention.

In certain embodiments, the shorter the length of a myomaker polypeptide, the fewer the modifications (e.g., substitutions) that can be made within the polypeptide while retaining, for example, a desired level of a chosen function. In some instances, longer domains can have a greater number of such changes while retaining, for example, a desired level of a chosen function. In other embodiments, a full-length polypeptide can have more tolerance for a fixed number of changes while retaining, for example, a desired level of a chosen function, as compared to a shorter length of that polypeptide.

The design of substitutions can take many forms, including but not limited to those described herein. In some embodiments, the hydropathic index of amino acids may be considered in designing substitutions. In the hydropathic index, each amino acid is assigned a hydropathic index on the basis of their hydrophobicity or charge characteristics, as follows: isoleucine (+4.5); valine (+4.2); Leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); or arginine (−4.5). In some instances, certain amino acids may be substituted for other amino acids having a similar hydropathic index. In making changes based upon the hydropathic index, the substitution of amino acids with hydropathic indices can be made with amino acids that have an index difference of no more than ±2, no more than ±1, or no more than ±0.5.

In some embodiments, substitutions can also be made based on hydrophilicity values. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids with hydrophilicity values can be made with amino acids that have a value of no more than ±2, no more than ±1, or no more than ±0.5.

A “conservative substitution” in an amino acid sequence or polypeptide indicates that a given amino acid residue is replaced by a residue having similar physiochemical characteristics (e.g., no more than ±1 when based on hydropathic index or no more than ±1 when base on hydrophilicity values). Examples of conservative substitutions include (a) substitution of one aliphatic residue for another with an aliphatic residue, (b) substitution of one of Ile, Val, Leu, or Ala for one another of Ile, Val, Leu, or Ala, (c) substitution of one of Gly, Ile, Val, Leu, or Ala for one another of Gly, Ile, Val, Leu, or Ala, (d) substitution of one polar residue for another polar residue, (e) substitution of one of Lys and Arg with another of Lys and Arg, (f) substitution of one of Glu and Asp with another of Glu and Asp, (g) substitution of one of Gln and Asn with another of Gln and Asn, (h) substitution of one hydroxyl or sulfur containing residue with another hydroxyl or sulfur containing residue, (i) substitution of one of Ser, Cys, Thr, or Met with another of Ser, Cys, Thr, or Met, (j) substitution of one aromatic residue for another with an aromatic residue, (k) substitution of one of Phe, Tyr, or Trp with another of Phe, Tyr, or Trp, (l) substitution of one basic residue for another basic residue, (m) substitution of one of His, Lys, or Arg with another of His, Lys, or Arg, (n) substitution of an acidic/amide residue with another acidic/amide residue, (o) substitution of one of Asp, Glu, Asn, or Gln with another of Asp, Glu, Asn, or Gln, (p) substitution of a residue with another residue of a similar size, and (q) substitution of one of Ala, Gly, or Ser with another of Ala, Gly, or Ser. In some embodiments, each amino acid in a hydrophobic region of a polypeptide can be substituted with conservative substitutions (e.g., any combination of conservative substitutions relating to hydrophobic residues).

While discussion has focused on amino acid changes, it will be appreciated that these changes may occur by alteration of the encoding DNA; taking into consideration also that the genetic code is degenerate and that two or more codons may code for the same amino acid. Tables A and B of amino acids and their codons are presented herein for use in such embodiments, as well as for other uses, such as in the design of probes and primers and the like.

Tables A and B. Amino acid designations and codon table

| Table A - Amino Add Designations | | | Table B - Codons for Amino Acids |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The term “functionally equivalent codon” is used herein to refer to codons that encode the same amino acid, such as the six codons for arginine or serine.

In certain instances, the nucleic acid molecule can be engineered to contain distinct sequences while at the same time retaining the capacity to encode a desired inventive polypeptide. In some embodiments, this can be accomplished owing to the degeneracy of the genetic code (i.e., the presence of multiple codons) which encode for the same amino acids. In other instances, it can be accomplished by including, adding, or excluding introns in the nucleic acid molecule.

In certain embodiments, a restriction enzyme recognition sequence can be introduced into a nucleic acid sequence while maintaining the ability of that nucleic acid molecule to encode a desired polypeptide. In other embodiments, a CRISPR system (e.g., a CRISPR system comprising one or more of guide RNA, crRNA, tracrRNA, sgRNA, DNA repair template, and Cas protein, such as but not limited to CRISPR/Cas9) can be used to introduce a nucleic acid molecule while maintaining the ability of that nucleic acid molecule to encode a desired polypeptide.

It will also be understood that amino acid sequences (e.g., polypeptides) and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological activity where polypeptide expression is concerned. The addition of terminal sequences particularly applies to nucleic acid sequences that may, for example, include various non-coding sequences flanking either of the 5′ or 3′ portions of the coding region or may include various internal sequences, (i.e., introns) which can occur within genes.

Some embodiments use synthesis of polypeptides in cyto, via transcription and translation of appropriate nucleic acid molecules (e.g., nucleic acid sequences as discussed herein). These polypeptides will include the twenty “natural” amino acids, and post-translational modifications thereof. In vitro peptide synthesis permits the use of modified or unusual amino acids. In some embodiments, the myomaker polypeptide encompasses modifications (e.g., one or more substitutions or one or more insertions) that include one or more modified or unusual amino acids. A table of exemplary, but not limiting, modified or unusual amino acids is provided in Table C.

TABLE C
Modified or Unusual Amino Acids

| Abbr. | Amino Acid |
|---|---|
| Aad | 2-Aminoadipic acid |
| BAad | 3-Aminoadipic acid |
| BAla | beta-alanine, beta-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| BAib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| Aile | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

The presently disclosed subject matter further includes a method of producing a myomaker polypeptide (e.g., a mutant myomaker polypeptide or a wt-myomaker polypeptide). Any suitable method can used to make the myomaker polypeptides including but not limited to expression through any suitable molecular biological technique (e.g., using a prokaryotic or eukaryotic expression system), isolation from a source in nature, or chemical synthesis. Eukaryotic expression systems include plant-based systems; insect cell systems via recombinant baculoviruses; whole insect systems via recombinant baculoviruses; genetically engineered yeast systems, including but not limited to Saccharomyces sp. and Picchia spp.; and mammalian cell systems, including but not limited to C2C12 cells, 10T ½ fibroblasts, NIH/3T3 fibroblasts, mesenchymal stem cells (MSCs), hematopoietic stem cells, Chinese hamster ovary cells or other cell lines commonly used for industrial scale expression of recombinant proteins. In some embodiments, useful plant-based expression systems can include transgenic plant systems. In some embodiments, useful plant-based expression systems can include transplastomic plant systems.

In some embodiments, a method of producing the myomaker polypeptide includes providing a host cell comprising a myomaker nucleic acid molecule, as disclosed herein, operatively linked to a promoter operable under conditions whereby the encoded myomaker polypeptide is expressed; and recovering the myomaker polypeptide from the host cell.

Dystrophin Polypeptides and Dystrophin Nucleic Acid Molecules

Some embodiments of the invention include compositions comprising the dystrophin polypeptide, the dystrophin nucleic acid molecule, or both, cells comprising the dystrophin polypeptide, the dystrophin nucleic acid molecule, or both, or using the dystrophin polypeptide, the dystrophin nucleic acid molecule, or both. In some embodiments, the dystrophin polypeptide is a microdystrophin polypeptide or a a minidystrophin polypeptide. The term “dystrophin polypeptide” encompasses “wt-dystrophin polypeptides” (i.e., dystrophin polypeptides found in nature without any purposely human-made modification) and “mutant dystrophin polypeptides” (e.g., with one or more modifications made to a wt-dystrophin polypeptide, such as any of the modifications disclosed above). In other embodiments, the dystrophin polypeptide has at least one amino acid modification relative to a wt-dystrophin polypeptide (e.g., any of those disclosed above, such as conservative substitutions). A wt-dystrophin polypeptide can, in some embodiments, be a dystrophin polypeptide from any animal including but not limited to a mammal, a rat, a cat, a rabbit, a human, a cow, a chicken, a turkey, a monkey, a tree shrew, a dog, a pig, a shrew, an elephant, or an opossum.

Nucleic acid molecules that encode for the dystrophin polypeptide are termed “dystrophin nucleic acid molecules.” In certain embodiments, the dystrophin nucleic acid molecule is included in a vector (e.g., a viral vector, a retroviral vector, a lentiviral vector, an adenoviral vector, an adeno-associated viral vector, a herpesviral vector, a chimeric viral vector, a plasmid, an expression vector, a conjugative vector, or a nonconjugative vector). In certain embodiments, the dystrophin nucleic acid molecule is in a cell, such as an insect cell (e.g., an Sf9 cell) or mammalian cell (e.g., a human cell, a rat cell a mouse cell, a muscle cell, a non-muscle cell, a myoblast, a fibroblast, a C2C12 cell, a 10T ½ fibroblast, a NIH/3T3 cell, a CHO cell, a mesenchymal stem cell (MSC), a hematopoietic stem cell, a blood cell, a bone marrow cell, or an adipose stem cell).

Cells Including Modified Cells

Some embodiments of the invention include cells such as modified cells. In certain embodiments, a modified cell is a cell that comprises one or more modifications of a cell, where at least one of the one or more modifications was implemented by a human (e.g., by human activity, either directly or indirectly). In some embodiments, the cell to be modified can be an unmodified cell or can be a cell that has been previously modified (e.g. modified as disclosed herein). A cell can be modified in any desired manner, including but not limited to (a) adding a nucleic acid molecule such as but not limited to one or more nucleic acid molecules disclosed herein (myomaker, dystrophin, or both), (b) adding one or more polypeptides, including but not limited to polypeptides disclosed herein, (c) expressing (e.g., overexpressing) one or more polypeptides (e.g., myomaker, dystrophin, or both), or (d) a combination thereof (e.g., expressing myomaker and overexpressing dystrophin). In some instances, a modified cell can result from a further modification of another modified cell.

Adding a nucleic acid molecule to modify a cell can be accomplished using any suitable method including but not limited to one or more of transformation (as used herein transfection methods are encompassed by the term transformation), viral transformation (e.g., using a viral vector, a retroviral vector, a lentiviral vector, an adenoviral vector, an adeno-associated viral vector, a herpesviral vector, a chimeric viral vector, a plasmid, a cosmid, an artificial chromosome, a bacteriophage, a virus, an animal virus, a plant virus, an expression vector, a conjugative vector, or a nonconjugative vector), injection, microinjection, electroporation, sonication, calcium ion treatment, calcium phosphate precipitation, PEG-DMSO treatment, DE-Dextran treatment, liposome mediated transformation, or a receptor mediated transformation. Adding a polypeptide to modify a cell can be accomplished using any suitable method including but not limited to one or more of injection, microinjection, electroporation, sonication, calcium ion treatment, calcium phosphate precipitation, PEG-DMSO treatment, DE-Dextran treatment, or liposome mediated. The added nucleic acid molecule can be part of a vector (e.g., a viral vector, a retroviral vector, a lentiviral vector, an adenoviral vector, an adeno-associated viral vector, a herpesviral vector, a chimeric viral vector, a plasmid, a cosmid, an artificial chromosome, a bacteriophage, an animal virus, a plant virus, an expression vector, a conjugative vector, or a nonconjugative vector), a plasmid, a cosmid, an artificial chromosome, a bacteriophage, a virus, an animal virus, or a plant virus. In some embodiments, the added nucleic acid molecule is exogenous; “exogenous” means (a) that the added nucleic acid molecule originates from outside of the cell (e.g., is foreign to the cell) or (b) that the added nucleic acid molecule can be found inside the cell, but the added nucleic acid molecule is placed in the cell where it is not normally found (e.g., a different part of the chromosome or on an added plasmid). In some embodiments, the added polypeptide is exogenous; “exogenous” in this context means that the added polypeptide originates from outside of the cell (e.g., is foreign to the cell).

The cell to be modified can be any suitable cell including but not limited to an insect cell (e.g., an Sf9 cell), a vertebrate cell, or a mammalian cell (e.g., a human cell, a rat cell a mouse cell, a muscle cell, a non-muscle cell, a myoblast, a fibroblast, a C2C12 cell, a 10T ½ fibroblast, a NIH/3T3 cell, a CHO cell, a mesenchymal stem cell (MSC), a hematopoietic stem cell, a blood cell, a bone marrow cell, a stem cell, or an adipose stem cell). In certain embodiments, an unmodified cell can be any suitable cell including but not limited insect cell, a vertebrate cell, or a mammalian cell (e.g., a human cell, a rat cell a mouse cell, a muscle cell, a non-muscle cell, a myoblast, a fibroblast, a NIH/3T3 cell, a CHO cell, a mesenchymal stem cell (MSC), a hematopoietic stem cell, a blood cell, a bone marrow cell, a stem cell, or an adipose stem cell).

In some embodiments, a modified cell can be but is not limited to a modified animal cell, a modified vertebrate cell, a modified mammalian cell, a modified human cell, a modified rat cell, a modified mouse cell, a modified muscle cell, a modified non-muscle cell, a modified myoblast, a modified fibroblast, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell. In other embodiments, the modified cell is a modified non-muscle cell (e.g., a modified fibroblast, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell).

In other embodiments, the modified cell is a non-muscle cell with a myomaker and/or dystrophin nucleic acid molecule added (e.g., where the myomaker and/or dystrophin nucleic acid molecule is exogenous), a stem cell with a myomaker and/or dystrophin nucleic acid molecule added (e.g., where the myomaker and/or dystrophin nucleic acid molecule is exogenous), a fibroblast with a myomaker and/or dystrophin nucleic acid molecule added (e.g., where the myomaker and/or dystrophin nucleic acid molecule is exogenous), a muscle cell with a myomaker and/or dystrophin nucleic acid molecule added (e.g., where the myomaker and/or dystrophin nucleic acid molecule is exogenous), a myoblast cell with a myomaker and/or dystrophin nucleic acid molecule added (e.g., where the myomaker and/or dystrophin nucleic acid molecule is exogenous), or a MSC cell with a myomaker and/or dystrophin nucleic acid molecule added (e.g., where the myomaker and/or dystrophin nucleic acid molecule is exogenous).

The modified cell can be prepared using any suitable method including but not limited to those disclosed herein or those found in L I et al. 2005, which is herein incorporated by reference in its entirety (L I et al. (2005) “Stable transduction of myogenic cells with lentiviral vectors expressing a minidystrophin” Gene Therapy, Vol. 12, pp. 1099-1108.) (e.g., using the lentiviral vector with a human CMV promotor or a murine stem cell virus promoter (MSCV)) to modify or partially modify a cell.

Compositions Including Pharmaceutical Compositions

One or more polypeptides (e.g., wt-myomaker polypeptide, mutant myomaker polypeptide, wt-dystrophin polypeptide, or mutant dystrophin polypeptide) or one or more myomaker or dystrophin nucleic acid molecules (e.g., in the form of a bare nucleic acid molecule, a vector, a virus, a plasmid or any suitable form) can be part of a composition and can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, or no more than about 99.99%, from about 0.0001% to about 99%, from about 0.0001% to about 50%, from about 0.01% to about 95%, from about 1% to about 95%, from about 10% to about 90%, or from about 25% to about 75%. In certain embodiments, cells, such as modified cells (e.g., as disclosed herein) can be part of the composition at any amount indicated herein (e.g., indicated above).

One or more polypeptides (e.g., wt-myomaker polypeptide, mutant myomaker polypeptide, wt-dystrophin polypeptide, or mutant dystrophin polypeptide) or one or more myomaker or dystrophin nucleic acid molecules (e.g., in the form of a bare nucleic acid molecule, a vector, a virus, a plasmid or any suitable form) can be purified or isolated in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%. In some embodiments, isolated or purified means that impurities (e.g., cell components or unwanted solution components if chemically synthesized) were removed by one or more of any suitable technique (e.g., column chromatography, HPLC, centrifugation, fractionation, gel, precipitation, or salting out).

Some embodiments of the present invention include compositions comprising one or more polypeptides (e.g., wt-myomaker polypeptide, mutant myomaker polypeptide, wt-dystrophin polypeptide, or mutant dystrophin polypeptide) or one or more myomaker or dystrophin nucleic acid molecules (e.g., in the form of a bare nucleic acid molecule, a vector, a virus, a plasmid or any suitable form). In certain embodiments, cells, such as modified cells (e.g., as disclosed herein) can be part of the composition at any amount indicated herein (e.g., indicated above). In certain embodiments, the composition is a pharmaceutical composition, such as compositions that are suitable for administration to animals (e.g., mammals, primates, monkeys, humans, canine, porcine, mice, rabbits, or rats). In some embodiments, there may be inherent side effects (e.g., it may harm the patient or may be toxic or harmful to some degree in some patients).

In some embodiments, one or more polypeptides (e.g., wt-myomaker polypeptide, mutant myomaker polypeptide, wt-dystrophin polypeptide, or mutant dystrophin polypeptide) or one or more myomaker or dystrophin nucleic acid molecules (e.g., in the form of a bare nucleic acid molecule, a vector, a virus, a plasmid or any suitable form) can be part of a pharmaceutical composition and can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, cells, such as modified cells (e.g., as disclosed herein) can be part of the pharmaceutical composition at any amount indicated herein (e.g., indicated above).

In some embodiments, the pharmaceutical composition can be presented in a dosage form which is suitable for the topical, subcutaneous, intrathecal, intraperitoneal, oral, parenteral, rectal, cutaneous, nasal, vaginal, or ocular administration route. In other embodiments, the pharmaceutical composition can be presented in a dosage form which is suitable for parenteral administration, a mucosal administration, intravenous administration, subcutaneous administration, topical administration, intradermal administration, oral administration, sublingual administration, intranasal administration, or intramuscular administration. The pharmaceutical composition can be in the form of, for example, tablets, capsules, pills, powders granulates, suspensions, emulsions, solutions, gels (including hydrogels), pastes, ointments, creams, plasters, drenches, delivery devices, suppositories, enemas, injectables, implants, sprays, aerosols or other suitable forms.

In some embodiments, the pharmaceutical composition can include one or more formulary ingredients. A “formulary ingredient” can be any suitable ingredient (e.g., suitable for the drug(s), for the dosage of the drug(s), for the timing of release of the drugs(s), for the disease, for the disease state, for the organ, or for the delivery route) including, but not limited to, water (e.g., boiled water, distilled water, filtered water, pyrogen-free water, or water with chloroform), sugar (e.g., sucrose, glucose, mannitol, sorbitol, xylitol, or syrups made therefrom), ethanol, glycerol, glycols (e.g., propylene glycol), acetone, ethers, DMSO, surfactants (e.g., anionic surfactants, cationic surfactants, zwitterionic surfactants, or nonionic surfactants (e.g., polysorbates)), oils (e.g., animal oils, plant oils (e.g., coconut oil or arachis oil), or mineral oils), oil derivatives (e.g., ethyl oleate, glyceryl monostearate, or hydrogenated glycerides), excipients, preservatives (e.g., cysteine, methionine, antioxidants (e.g., vitamins (e.g., A, E, or C), selenium, retinyl palmitate, sodium citrate, citric acid, chloroform, or parabens, (e.g., methyl paraben or propyl paraben)), or combinations thereof. In some embodiments, the concentration of any individual formulary ingredient in a composition (e.g., pharmaceutical composition) can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, the concentration of at least one formulary ingredient is not that same as that found in the natural system in which the polypeptide (e.g., wt-myomaker polypeptide or wt-dystrophin polypeptide) is found. In some embodiments, the concentration of at least one formulary ingredient is not that same as that found in one or more natural systems (e.g., any natural system found in nature) in which the nucleic acid molecule which encodes a polypeptide (e.g., wt-myomaker polypeptide or wt-dystrophin polypeptide) is found.

In certain embodiments, pharmaceutical compositions can be formulated to release the active ingredient (e.g., wt-myomaker polypeptide, wt-dystrophin polypeptide, or modified cell) substantially immediately upon the administration or any substantially predetermined time or time after administration. Such formulations can include, for example, controlled release formulations such as various controlled release compositions and coatings.

Other formulations (e.g., formulations of a pharmaceutical composition) can, in certain embodiments, include those incorporating the drug (or control release formulation) into food, food stuffs, feed, or drink.

Methods of Using Cells Including Modified Cells

Some embodiments of the invention include methods of using cells, such as modified cells. Some embodiments of the invention include methods for administering one or more modified cells (e.g., as disclosed herein) to an animal.

In some embodiments, the unmodified cell can be any suitable cell including but not limited to an insect cell (e.g., an Sf9 cell), a vertebrate cell, or a mammalian cell (e.g., a human cell, a rat cell a mouse cell, a muscle cell, a non-muscle cell, a myoblast, a fibroblast, a C2C12 cell, a 10T ½ fibroblast, a NIH/3T3 cell, a CHO cell, a dendritic cell, a cancer cell, a mesenchymal stem cell (MSC), a hematopoietic stem cell, a blood cell, a bone marrow cell, a stem cell, or an adipose stem cell).

In some embodiments, the modified cell can be any suitable cell including but not limited to a modified animal cell, a modified vertebrate cell, a modified mammalian cell, a modified human cell, a modified rat cell, a modified mouse cell, a modified muscle cell, a modified non-muscle cell, a modified myoblast, a modified fibroblast, a C2C12 cell, a modified C2C12 cell, a 10T ½ fibroblast, a modified 10T ½ fibroblast, a modified NIH/3T3 cell, a modified CHO cell, a modified dendritic cell, a modified cancer cell, a modified mesenchymal stem cell (MSC), a modified hematopoietic stem cell, a modified blood cell, a modified bone marrow cell, a modified stem cell, or a modified adipose stem cell. In other embodiments, the modified cell can be a modified cell that is a modified non-muscle cell (e.g., a modified fibroblast, a 10T ½ fibroblast, a modified 10T ½ fibroblast, a modified NIH/3T3 cell, a modified CHO cell, a modified dendritic cell, a modified cancer cell, a modified mesenchymal stem cell (MSC), a modified hematopoietic stem cell, a modified blood cell, a modified bone marrow cell, a modified stem cell, or a modified adipose stem cell). In some embodiments, the modified cell is an MSC cell that expresses myomaker, expresses or overexpresses dystrophin (e.g., microdystrophin or minidystrophin), or a combination thereof.

The administering of the one or more modified cells in the method can occur by any suitable manner, such as but not limited to those disclosed herein. For example, the administering can be accomplished by implanting, injecting, or grafting the one or more modified cells in an animal. Any suitable administration route can be used, including but not limited to those disclosed herein.

Animals include but are not limited to mammals, primates, monkeys (e.g., macaque, rhesus macaque, or pig tail macaque), humans, canine, feline, bovine, porcine, avian (e.g., chicken), mice, rabbits, and rats. As used herein, the term “subject” refers to both human and animal subjects.

In certain embodiments, the method to administer can be part of a treatment of a disease. In some embodiments, the disease can be any disease, such as but not limited to, diseases where cells underexpress dystrophin (e.g., microdystrophin or minidystrophin), do not express dystrophin (e.g., microdystrophin or minidystrophin), express a defective version of dystrophin (e.g., microdystrophin or minidystrophin), or a combination thereof. In some embodiments, the disease can be a non-muscle-related disease, such as but not limited to, non-muscle diseases where cells underexpress dystrophin (e.g., microdystrophin or minidystrophin), do not express dystrophin (e.g., microdystrophin or minidystrophin), express a defective version of dystrophin (e.g., microdystrophin or minidystrophin), or a combination thereof. In some embodiments, the disease can be a muscle-related disease, such as but not limited to, muscle-related diseases where cells underexpress dystrophin (e.g., microdystrophin or minidystrophin), do not express dystrophin (e.g., microdystrophin or minidystrophin), express a defective version of dystrophin (e.g., microdystrophin or minidystrophin), or a combination thereof. In certain embodiments, the treated disease can be a myopathy, muscular dystrophy, amyotrophic lateral sclerosis (ALS or also called Lou Gehrig's disease), glycogen storage disease type II (also called Pompe disease), rhabdomyosarcoma (RMS), sarcopenia, or a combination thereof. In some embodiments, the disease can be cancer. As used herein, the term “treating” (and its variations, such as “treatment”) is to be considered in its broadest context. In particular, the term “treating” does not necessarily imply that an animal is treated until total recovery. Accordingly, “treating” includes amelioration of the symptoms, relief from the symptoms or effects associated with a condition, decrease in severity of a condition, or preventing, preventively ameliorating symptoms, or otherwise reducing the risk of developing a particular condition. As used herein, reference to “treating” an animal includes but is not limited to prophylactic treatment and therapeutic treatment. Any of the methods or compositions (e.g., pharmaceutical compositions) described herein can be used to treat an animal.

In yet other embodiments, the delivery of one or more modified cells can occur by any suitable administration route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, depot injection, subcutaneous administration, topical administration, intradermal administration, oral administration, sublingual administration, intranasal administration, or intramuscular administration (e.g., intramuscular injection). In certain embodiments, the delivery comprises an injection or an intramuscular injection. In certain embodiments, the delivery comprises an injection comprising the modified cell (e.g., in a composition or in a pharmaceutical composition). In other embodiments, the delivery comprises an intramuscular injection comprising the modified cell (e.g., in a composition or in a pharmaceutical composition).

In still other embodiments, the treating can further comprise one or more of the administering steps.

The presently-disclosed subject matter is further illustrated by the following specific but non-limiting examples. The following examples may include compilations of data that are representative of data gathered at various times during the course of development and experimentation related to the present invention.

### EXAMPLES

Materials and Methods

Animals

C57BL/6 (WT), mdx^{4cv} (Jackson Laboratory #002378), Myomaker^{LacZ/loxP}; Pax7^{CreERT2}(myomaker^{scKO}), Rosa26^{mTmG} (Jackson Laboratory #007676), Rosa26^{tdTomato} (Jackson Laboratory #007905), myl1^{Cre/+} (Jackson Laboratory #024713) mice were used as a source of cells and/or a recipient in cell transplantation studies. Myomaker^{scKO} mice were generated as described previously (MILLAY et al. (2014) “Myomaker is essential for muscle regeneration” Genes Dev, Vol. 28, pp. 1641-1646). Tamoxifen (Sigma-Aldrich) was dissolved in corn oil with 10% ethanol at the concentration of 25 mg/mL and intraperitoneally administered at a dose of 0.075 mg/kg/day for 5 days. All animal procedures were approved by Cincinnati Children's Hospital Medical Center's Institutional Animal Care and Use Committee, and conducted in accordance with AAALAC guidelines.

Cell Preparation

WT MSCs were generated as described previously (GONZALEZ-NIETO et al. (2012) “Connexin-43 in the osteogenic BM niche regulates its cellular composition and the bidirectional traffic of hematopoietic stem cells and progenitors” Blood, Vol. 119, pp. 5144-5154). Briefly, bone marrow cells were plated on fibronectin-coated wells (Corning) in Iscove modified Dulbecco medium (IMDM) supplemented with 20% of MSC stimulatory supplements (StemCell Technologies), 100 μM 2-mercaptoethanol, 100 IU/mL penicillin, 0.1 mg/mL streptomycin, 2 mM L-glutamine, 10 ng/mL human platelet-derived growth factor (PDGF)—BB, and 10 ng/mL recombinant mouse epidermal growth factor (rM-EGF). Adherent clusters were grown for a minimum of five passages and macrophage depletion was assessed by flow cytometry. WT MSCs were maintained in high-glucose DMEM (HyClone) supplemented with 10% bovine growth serum (BGS, HyClone) and penicillin/streptomycin.

mTomato CBSCs were isolated from Rosa26^{mTmG} mice as described by others (DURAN et al., (2013) “Bone-derived stem cells repair the heart after myocardial infarction through transdifferentiation and paracrine signaling mechanisms” Circ Res, Vol. 113, pp. 539-552). This method was modified for isolation of CBSCs from myl1^{Cre/+} and WT mice. Femurs and tibias were collected and crunched with a mortar and pestle after removing epiphyses. Crunched bones were washed 5 times with phosphate buffered saline (PBS) to remove marrow cells and minced into ˜2 mm fragments with a scalpel. After incubation in low-glucose DMEM (Invitrogen) containing 0.3% collagenase type I (Invitrogen) for 1.5 h at 37° C., both bone fragments and cells were plated in low-glucose DMEM supplemented with 20% MSC stimulatory supplements, 30% Ham F10 (Invitrogen), 100 μM 2-mercaptoethanol, 10 ng/mL mouse PDGF-BB (ProSpec), 10 ng/mL mouse EGF (Novus Biologicals), 2.5 ng/mL human bFGF (Invitrogen). After seven days, the bone fragments were discarded and the cells were trypsinized and expanded. CBSCs were maintained in low-glucose DMEM supplemented with 15% fetal bovine serum (FBS), 40% Ham F10, 10 ng/mL mouse EGF, 2.5 ng/mL human bFGF, and penicillin/streptomycin.

To obtain TTFs, adult tails from WT mice were skinned and cut into small pieces with a razor blade. The tail explants were plated on 100 mm culture dishes with high-glucose DMEM containing 10% BGS and 1% penicillin/streptomycin, and the media was changed every other day. Fibroblasts were allowed to migrate out of the tail explants for 7 to 10 days and then trypsinized and plated for viral transduction.

Primary myoblasts were isolated from WT, myomaker^{scKO} or mdx^{4cv} mice as described previously (MILLAY et al (2016) “Structure-function analysis of myomaker domains required for myoblast fusion” Proc Natl Acad Sci USA, Vol. 113, pp. 2116-2121). Cells were plated on collagen-coated plates, maintained in growth media (15% FBS, 40% Ham F10, and 2.5 ng/mL human bFGF in low-glucose DMEM with penicillin/streptomycin), and differentiated in differentiation media (2% horse serum in high-glucose DMEM with penicillin/streptomycin). WT and myomaker primary myoblasts were infected with GFP or dsRED retrovirus as described below for the infection of non-muscle cells.

In Vitro Heterologous Fusion

Non-muscle cells were transduced with myomaker and/or GFP retrovirus as described previously (MILLAY et al (2016) “Structure-function analysis of myomaker domains required for myoblast fusion” Proc Natl Acad Sci USA, Vol. 113, pp. 2116-2121). Briefly, plasmid DNA was transfected using FuGENE6 (Roche) into Platinum E Cells (Cell Biolabs), and viral media was collected forty-eight hours after transfection. After addition of polybrene (Sigma), the viral supernatant was incubated on the target cells for eighteen hours. The transduction efficiency of all non-muscle cells, as assessed by immunostaining with a custom generated myomaker antibody, was between 95-100% for all infections (unpublished observations). Primary myoblasts were plated on a collagen-coated plate at the density of 37,500 cells/cm², and non-muscle cells were added the next day at the density of 6,250 cells/cm² and cultured in differentiation media. After five days, the cells were fixed in 4% paraformaldehyde (PFA)/PBS and permeabilized with 0.2% TritonX/PBS followed by blocking with 3% bovine serum albumin (BSA)/PBS, and then incubated with anti-myosin heavy chain (1:1000, clone MF20, R&D Systems) and/or anti-dystrophin antibodies (1:1000, #ab15277, Abcam) overnight at 4° C. followed by incubation with Alexa-Flour-secondary antibodies (1:1000).

Cell Transplantation

Cells were trypsinized and resuspended in PBS at the concentration of 2×10⁷ cells/mL, and kept on ice. Twenty-five μL of the cell suspension (500,000 cells) was loaded into a Gastight Hamilton syringe equipped with a 30 gauge needle and the needle was longitudinally inserted from the distal to proximal end of a tibialis anterior (TA) muscle. Injection was performed by five consecutive motions per muscle, which consisted of a 5 μL injection approximately every 1 mm as the needle was removed. Where indicated, TAs were injured by injection of 10 μM cardiotoxin (50 μL) twenty-four hours before transplantation. For trichostatin A (TSA) (ApexBio) treatment, an osmotic pump (#0000298, Durect) was filled with 50% DMSO/15% ethanol containing 6 mg/mL TSA and implanted subcutaneously. Cells were also treated with 0.1 μM TSA for 24 h before transplantation. For intracardiac cell delivery, the heart was exposed via left thoracotomy and 5×10⁴ GFP⁺ myomaker⁺ MSCs (suspended in 21 μL of sterile saline) were injected with a 33 g Hamilton syringe into three defined areas along the anterior wall of the left ventricle.

Muscle Histology

Hindlimbs were dissected with TAs attached to the bone and immersed in 4% PFA/PBS for 1-2 h at 4° C. A subset of muscles was imaged in whole-mount with a Zeiss Stereomicroscope to visualize tdTomato before removing the bone. The TA muscles were removed from the bone, cut into two pieces at the mid-belly and immersed in 2% PFA/PBS overnight at 4° C., and then placed in 30% sucrose/PBS at 4° C. After 1-2 days, the muscles were embedded in O.C.T., frozen, and 10 μm sections were collected. The sections were treated with permeabilizing/blocking solution (1% BSA, 1% heat-inactivated goat serum, 0.025% Tween20, and 0.2% TritonX-100 in PBS) for 1 h at room temperature and then incubated with anti-dystrophin (1:200) and/or anti-laminin-2 (1:500, #L-0633, Sigma-Aldrich) antibodies overnight at 4° C. The sections were incubated with Alexa Fluor-secondary antibodies (1:1000) for one hour at room temperature and mounted with VectaShield containing DAPI (Vector Laboratories). For analysis of fusion in cardiac tissue, hearts were arrested in diastole via intracardiac injection of ice cold 1M KCl and perfused with 4% PFA/PBS. Following four hours of fixation in PFA/PBS, hearts were washed twice with PBS and cryoprotected in 30% sucrose/PBS overnight and 5 μm cryosections were collected. All immunostaining in vitro and in vivo were visualized with a Nikon Eclipse Ti inverted microscope with A112 confocal running NIS Elements and images were analyzed with Fiji (SCHINDELIN et al. (2012) “Fiji: an open-source platform for biological-image analysis” Nat Methods, Vol. 9, pp. 676-682).

RNA Analysis

Cells were lysed and total RNA was extracted using RNAqueous®-Micro Kit (#AM1931, Invitrogen) and treated with DNase I. cDNA was synthesized using MultiScribe™ reverse transcriptase with random hexamer primers (Applied Biosystems). Myomaker expression was assessed using standard quantitative PCR approaches with PowerUp™ SYBR® Green Master Mix (Applied Biosystems). Analysis was performed on a 7900HT fast real-time PCR machine (Applied Biosystems) with the following primers: forward, ATCGCTACCAAGAGGCGTT (SEQ ID NO:17); reverse, CACAGCACAGACAAACCAGG (SEQ ID NO:18). Results were normalized using GAPDH with the following primers: forward, TGCGACTTCAACAGCAACTC (SEQ ID NO:19); reverse, GCCTCTCTTGCTCAGTGTCC (SEQ ID NO:20). Cre expression was assessed using standard PCR and gel electrophoresis with the following primers: forward, AGGTTCGTTCACTCATGGA (SEQ ID NO:21); reverse, TCGACCAGTTTAGTTACCC (SEQ ID NO:22). GAPDH expression was assessed as a reference gene with the same primers as noted above.

Quantification and Statistical Analysis

For quantitative assessment of in vivo fusion, the number of GFP⁺ or mTomato⁺ myofibers in a representative section of each muscle was manually counted and presented as mean±SEM. The data were analyzed with an unpaired Student's t-test with GraphPad Prism 6 software. A value of p<0.05 was considered statistically significant.

Results

Expression of Myomaker in MSCs Induces Fusion with Muscle

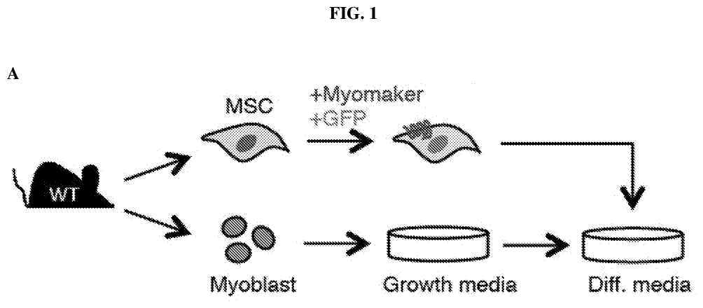

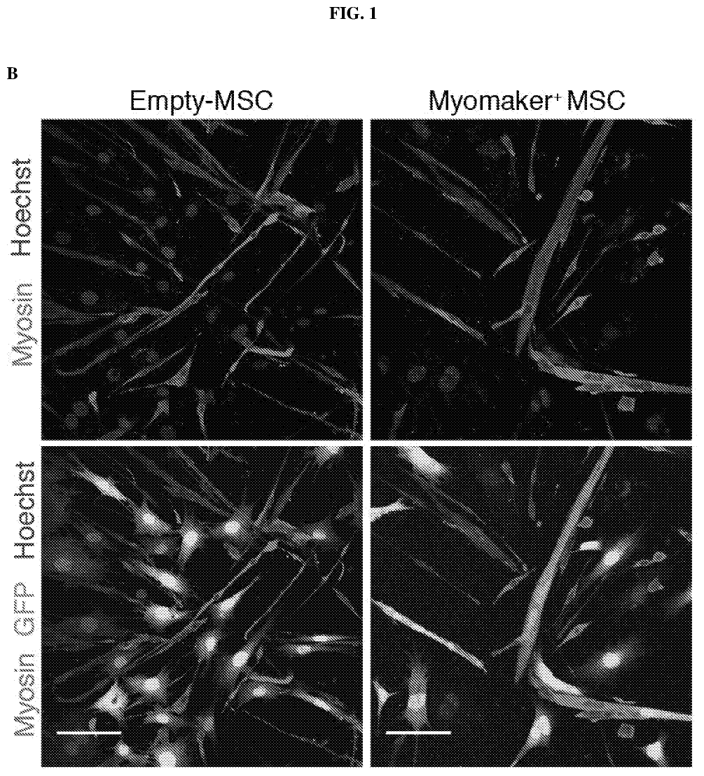

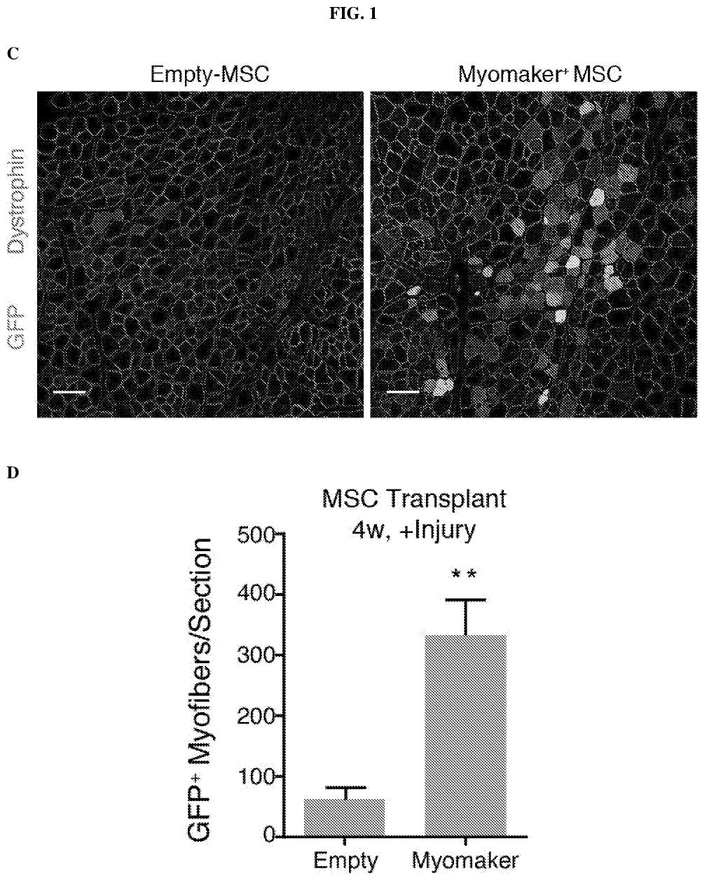

Myomaker-expressing 10T ½ fibroblasts fuse to primary myoblasts. To determine if MSCs are amenable to myomaker-mediated fusion we co-infected mouse MSCs with myomaker and GFP retroviruses. As a control, we also co-infected MSCs with empty and GFP retroviruses. GFP⁺ myomaker⁺ MSCs were then mixed with primary myoblasts from wild type (WT) mice and differentiated for five days (FIG. 1A). Myoblasts were tracked through immunostaining with an antibody to myosin, a marker of muscle differentiation. We observed GFP myosin⁺ structures when myoblasts were mixed with myomaker⁺ MSCs indicating fusion between these cell populations (FIG. 1B). We did not observe GFP⁺ myosin⁻ multi-nucleated cells indicating myomaker⁺ MSCs did not fuse to each other. GFP⁺ myosin⁺ cells were not readily detected in cultures containing empty-MSCs and myoblasts (FIG. 1B). We next tested whether myomaker⁺ MSCs were able to fuse to muscle in vivo. The tibialis anterior (TA) was injured with cardiotoxin (CTX) and the next day 500,000 GFP⁺ myomaker⁺ MSCs were transplanted into the TA. The presence of GFP within dystrophin⁺ myofibers indicates fusion of MSCs with muscle and was evaluated 4 weeks after transplant. Minimal GFP⁺ myofibers were detected after transplantation of Empty-MSCs, however an increase in heterologous fusion was observed with myomaker⁺ MSCs (FIG. 1C). Quantification of the number of GFP⁺ myofibers per section revealed a 5-fold increase in fusion of myomaker⁺ MSCs compared to empty-MSCs (FIG. 1D). These results demonstrate that myomaker-expressing MSCs are fusion competent and that muscle is susceptible to heterologous fusion in an in vivo transplantation setting.

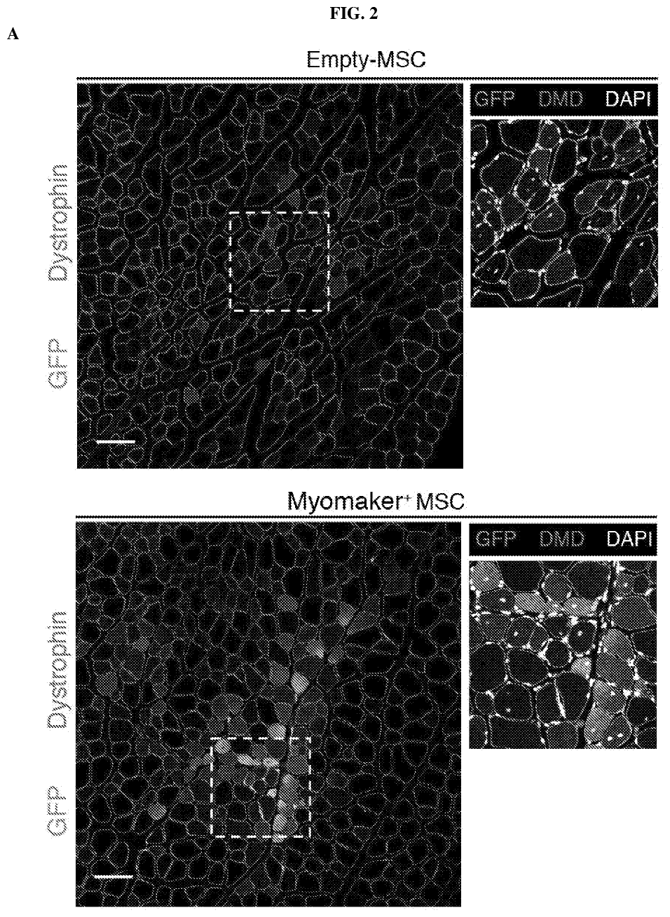

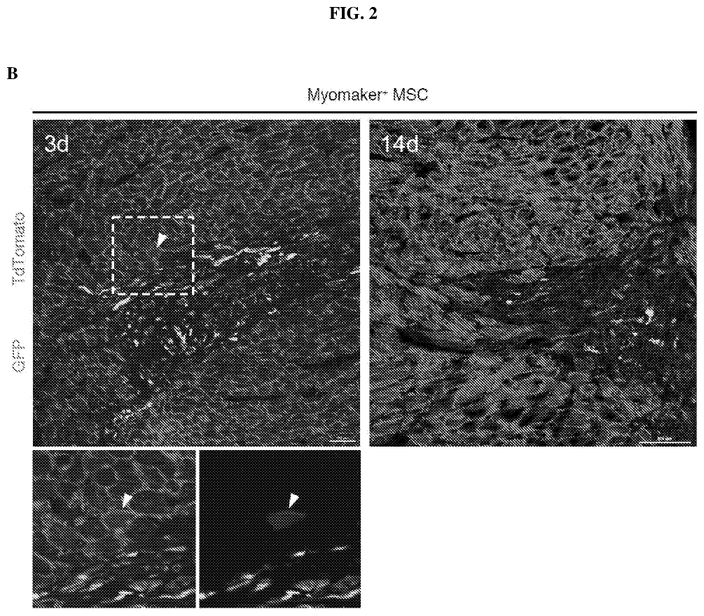

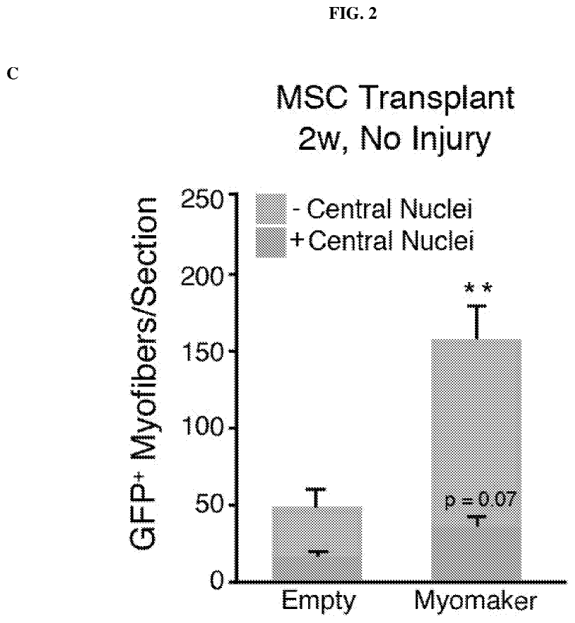

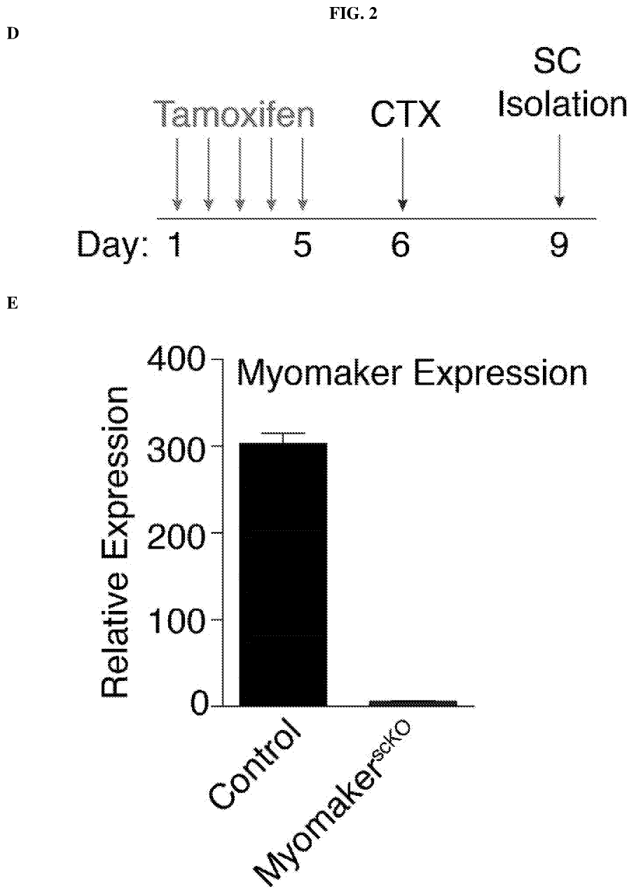

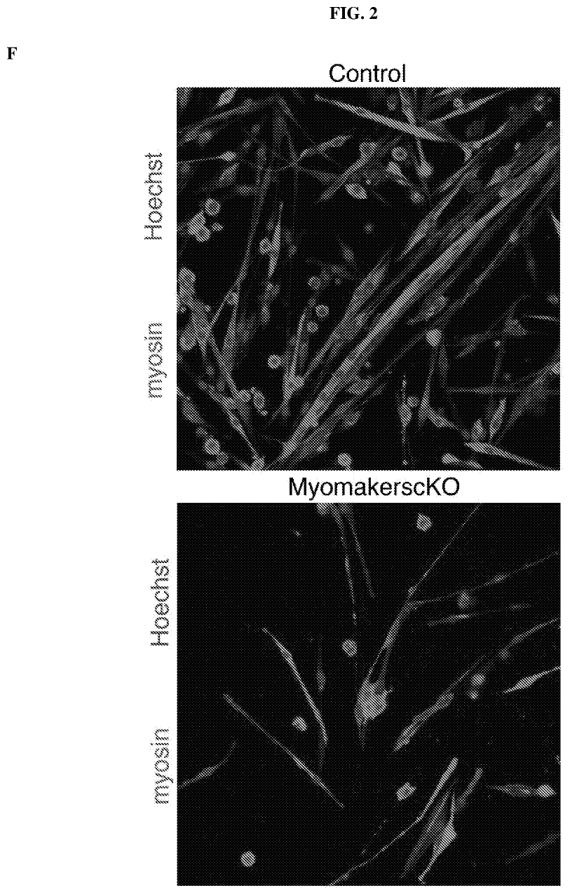

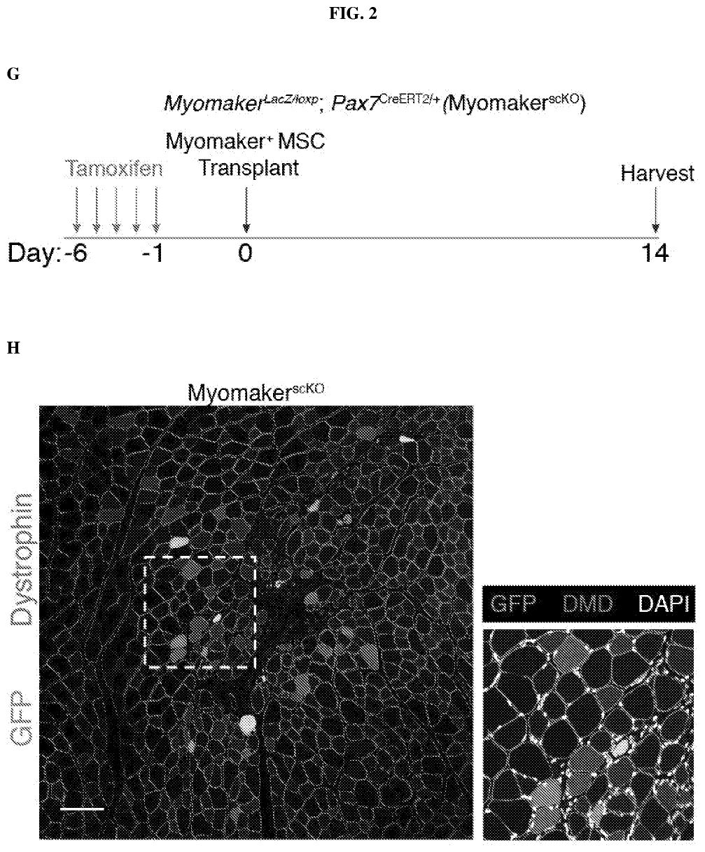

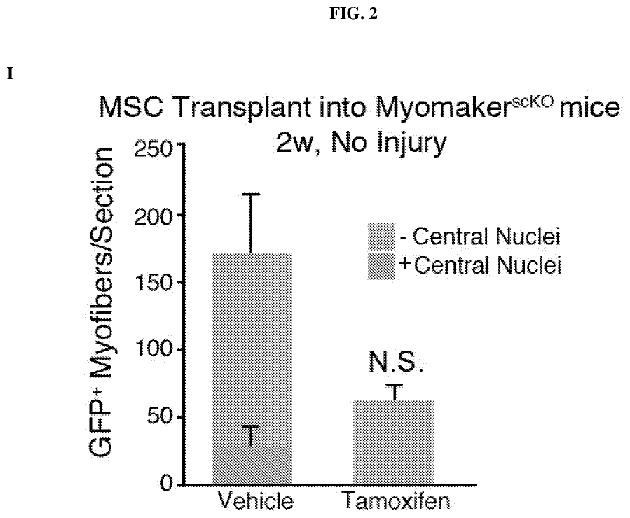

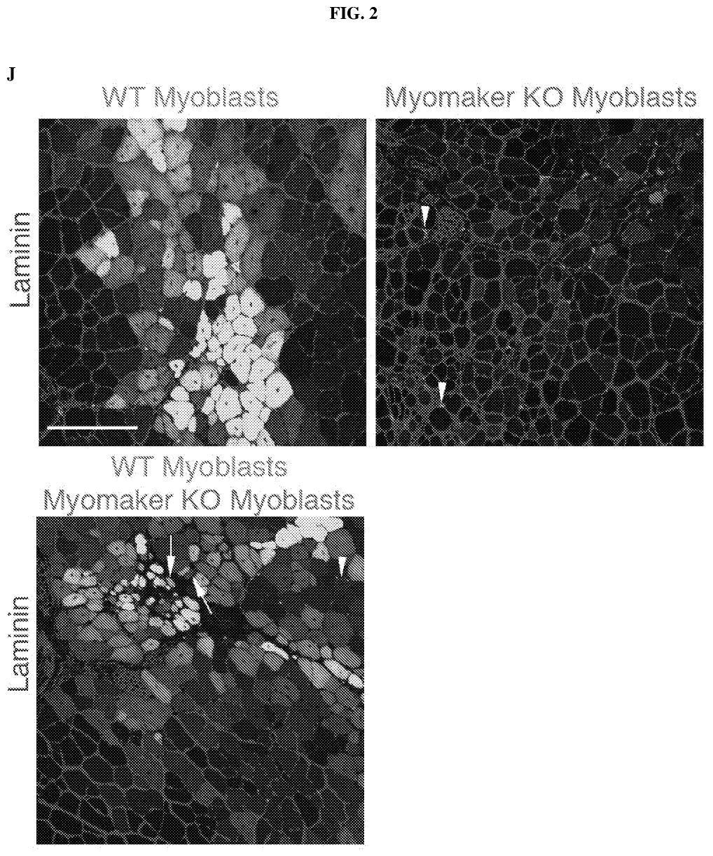

We next assessed if myomaker⁺ MSCs were able to fuse to muscle in the absence of injury, which then would suggest heterologous fusion could be utilized in non-dystrophic disease settings where satellite cells are not activated. Injection of myomaker⁺ MSCs into uninjured TAs resulted in fusion with muscle as depicted by GFP⁺ dystrophin⁺ myofibers (FIG. 2A). The ability of myomaker to fuse non-muscle cells to muscle was specific to skeletal muscle since transplantation of myomaker⁺ MSCs into cardiac tissue did not result in extensive fusion (FIG. 2B). Central nuclei can be a hallmark of regenerating muscle fibers and therefore can be a surrogate for muscle progenitor activity. While the recipient muscles in this experiment were not injured with CTX, we speculate that the central nuclei observed is due to minor injury from the needle during cell transplantation. No obvious differences in total (GFP⁺ or GFP⁻) central nucleated myofibers were detected after transplantation of empty MSCs or myomaker⁺ MSCs, indicating myomaker⁺ MSCs do not enhance damage resulting from the needle injury (FIG. 2A, insets). However, GFP⁺ fibers with and without central nuclei were increased by 2- and 4-fold, respectively, in myomaker⁺ MSCs compared to empty-MSCs, suggesting that myomaker confers fusion ability with mature myofibers and activated myoblasts (FIG. 2C).

Because transplantation of cells can induce muscle injury due to insertion of the needle, we inactivated the fusion ability of endogenous satellite cells through genetic deletion of myomaker to determine if myomaker⁺ MSCs were able to fuse directly to myofibers. Conditional deletion of myomaker in satellite cells was achieved by utilizing mice containing a myomaker targeted allele (myomaker^{LacZ/loxP}) and a Pax7^{CreERT2/+} allele that expresses Cre specifically in satellite cells. To make certain this approach was sufficient to generate myoblasts unable to fuse, we treated Myomaker^{Lac/loxP}; Pax7^{CreERT2/+} mice with either vehicle (control) or tamoxifen (myomaker^{scKO}), and isolated satellite cells from muscle three days after CTX injury. Myomaker expression was reduced in myomaker^{scKO} myoblasts and these cells did not appear to fuse, highlighting the utility of this system to block fusion of satellite cells (FIGS. 2D, 2E, and 2F). Myomaker^{LacZ/loxP}; Pax7^{CreERT2/+} mice were then treated with either vehicle or five doses of tamoxifen to delete myomaker in satellite cells and then transplanted with myomaker⁺ MSCs (FIG. 2G). Myomaker⁺ MSCs displayed fusion competency even after endogenous satellite cells were rendered fusion-incompetent suggesting they were able to fuse with myofibers (FIG. 2H). Quantification of fusion events with and without central nuclei indicates that myomaker⁺ MSCs indeed fuse to myofibers in the absence of satellite cell fusion activity (FIG. 2I). A non-statistically significant reduction in GFP⁺ myofibers without central nuclei was observed in tamoxifen-treated samples compared to vehicle. This reduction may suggest that the number of centrally nucleated fibers is an underestimation and that some fibers without central nuclei may contain central nuclei above or below this plane of section.

We have demonstrated that cells not expressing myomaker (empty-MSCs) are able to fuse with muscle, which suggests that fusion in vivo does not absolutely require myomaker on both cells. To further evaluate this concept, we generated WT-GFP myoblasts and myomaker KO-dsRED myoblasts and transplanted them into WT mice after CTX-induced injury. Compared to myomaker KO-dsRED myoblasts, WT-GFP myoblasts exhibited greater engraftment potential (FIG. 2J). Moreover, we observed an increased number of small mononuclear dsRED⁺ cells indicating these cells are unable to fuse (FIG. 2J). We also transplanted WT-GFP myoblasts and myomaker KO-dsRED myoblasts together and observed predominantly GFP⁺ muscle fibers and not dsRED⁺ fibers, suggesting WT-GFP myoblasts are more fusion competent (FIG. 2J). Overall, these data indicate that both myoblast-myoblast fusion and non-muscle-myoblast fusion can occur in vivo during muscle regeneration if at least one of the two cells expresses myomaker.

Dystrophin Reprogramming of MSC Nuclei after Heterologous Fusion

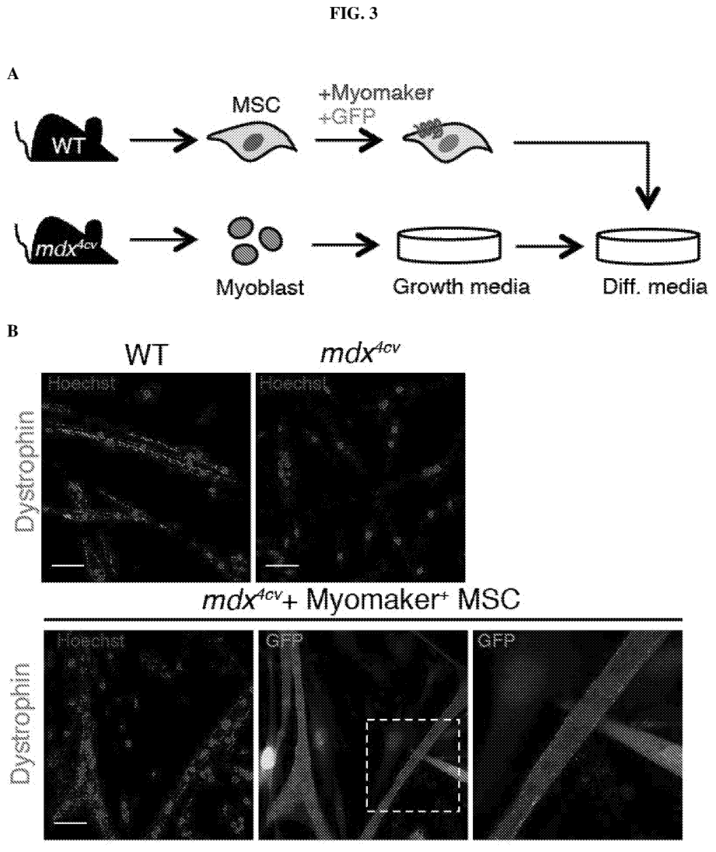

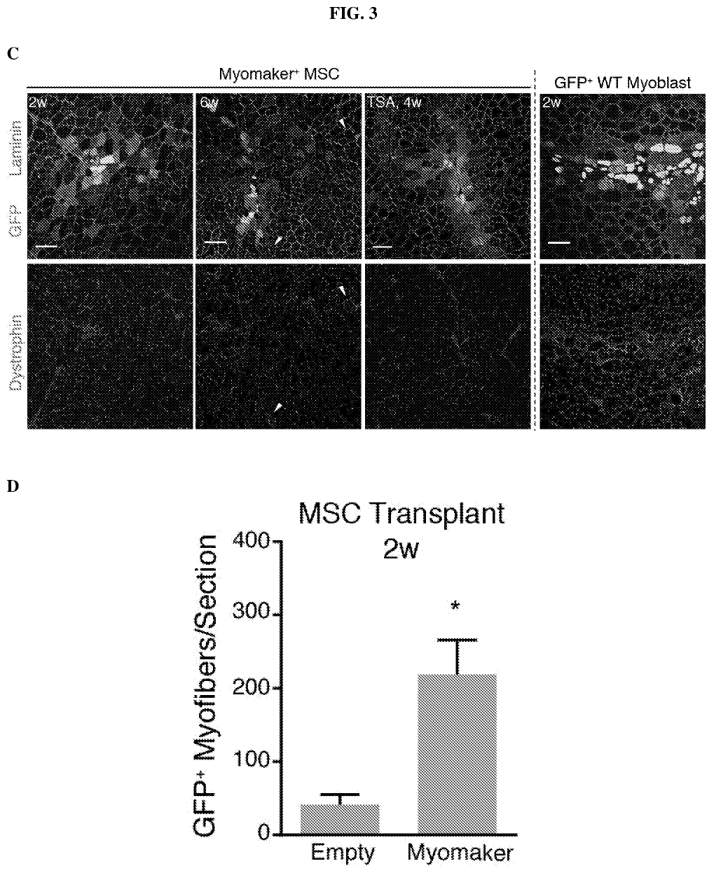

Since myomaker enhances fusion of MSCs, we evaluated if MSC nuclei were susceptible to reprogramming and able to restore dystrophin expression in mdx^{4cv} myotubes. We isolated myoblasts from mdx^{4cv} mice and co-cultured them with GFP⁺ myomaker⁺ MSCs (FIG. 3A). After five days of differentiation, we evaluated fusion and dystrophin expression in these cultures. As expected, dystrophin was detected in myotubes derived from WT myoblasts but not mdx^{4cv} myotubes (FIG. 3B). We observed fusion of myomaker⁺ MSCs with mdx^{4cv} myoblasts and dystrophin expression along the membrane in some chimeric myotubes, however most GFP⁺ myofibers were still dystrophin⁻ (FIG. 3B). Since mdx^{4cv} mice do not express dystrophin, the dystrophin observed in these myotubes likely originates from WT MSC nuclei indicating reprogramming. To determine if nuclear reprogramming also occurs in vivo we transplanted myomaker⁺ MSCs into the TAs of mdx^{4cv} mice without injury and assayed for fusion and reprogramming at two weeks and six weeks post-transplant. Myomaker⁺ MSCs fused to dystrophic myofibers, however we did not observe detectable levels of dystrophin either two weeks or six weeks after transplant (FIG. 3C). GFP⁺ myoblasts from WT mice were also transplanted into mdx^{4cv} mice for use as a positive control for dystrophin restoration (FIG. 3C). Quantification of GFP⁺ myofibers per section revealed that myomaker⁺ MSCs fused to approximately 200 mdx^{4cv} myofibers per section (FIG. 3D). We also treated mice with the HDAC inhibitor TSA as a means to globally enhance transcriptional activity, however this approach also did not result in dystrophin restoration (FIG. 3C). These results indicate that myomaker⁺ MSCs appear to be refractory to detectable levels of dystrophin reprogramming.

Fusion and Nuclear Reprogramming of Myomaker-CBSCs and Myomaker-TTFs

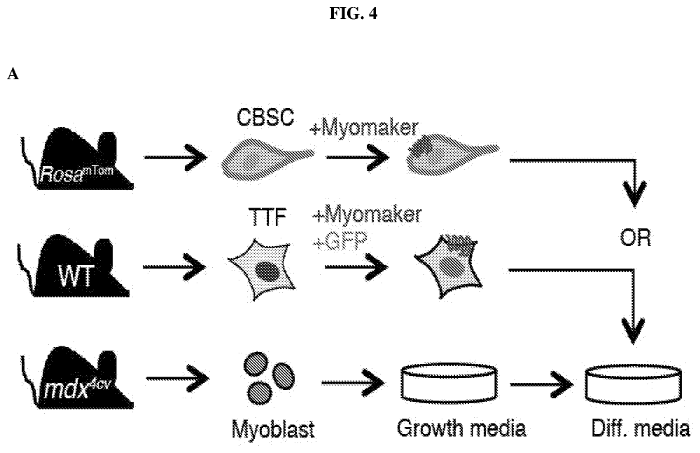

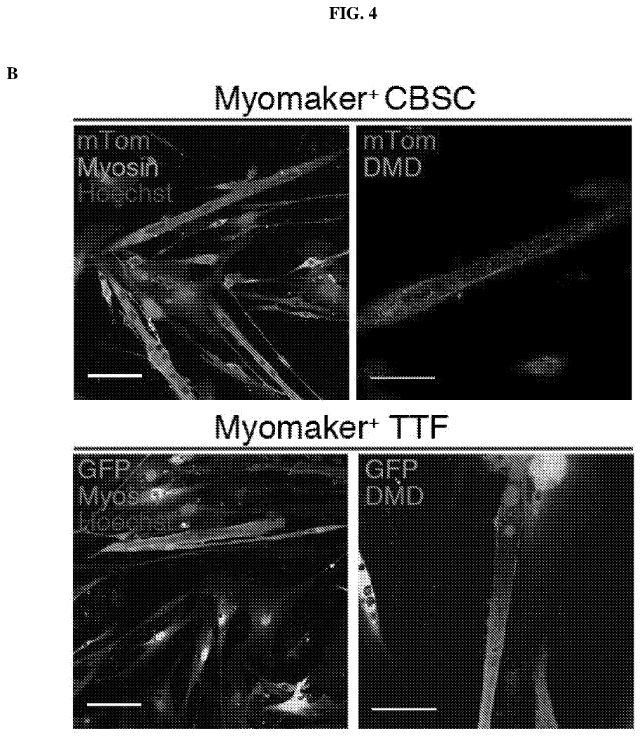

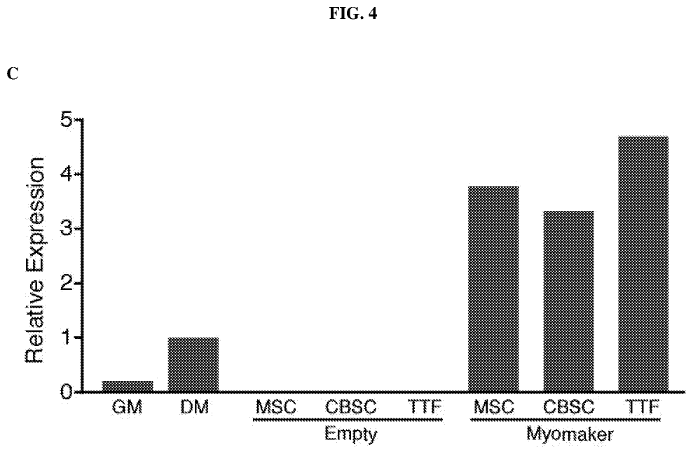

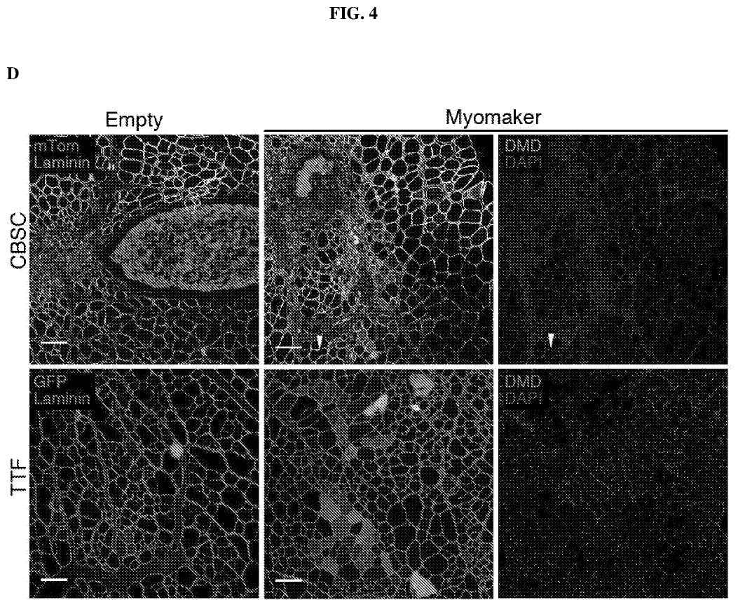

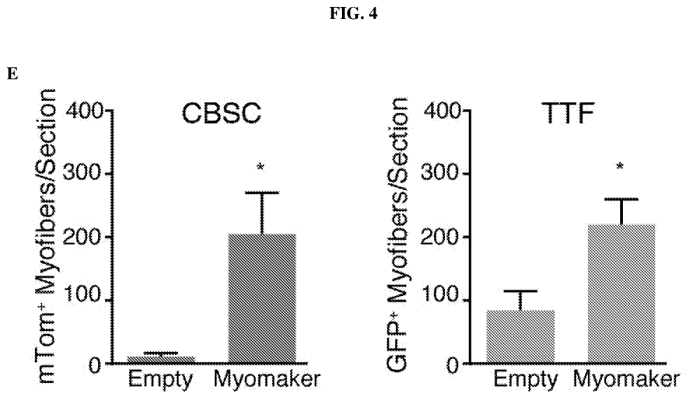

We also sought to determine if myomaker-mediated heterologous fusion and lack of functional reprogramming was restricted to MSCs or a similar phenomenon occurs in other heterologous cells. We tested TTFs because they have exhibited reprogramming in classical heterokaryon experiments and also CBSCs because they are similar to MSCs, although they may be in a more primitive state. We mixed either GFP⁺ myomaker⁺ TTFs or membrane Tomato (mTom)⁺ myomaker⁺ CBSCs with mdx^{4cv} myoblasts (FIG. 4A), and successfully detected fusion and dystrophin reprogramming in vitro (FIG. 4B). Myomaker levels in MSCs, CBSCs, and TTFs were comparable (FIG. 4C). Both cell types were then transplanted into TAs of mdx^{4cv} mice without injury and fusion was observed (FIG. 4D). Myomaker⁺ TTFs and myomaker⁺ CBSCs fused at similar levels to mdx^{4cv} muscle compared to myomaker⁺ MSCs (FIG. 4E). Similar to what was observed with myomaker⁺ MSCs, we did not detect dystrophin restoration in mdx^{4cv} muscle after fusion with either myomaker⁺ TTFs or myomaker⁺ CBSCs (FIG. 4D). Dystrophin was also not detected seven weeks after transplantation of CBSCs (data not shown). Taken together, myomaker enhances fusion of multiple non-muscle cell types with muscle but dystrophin expression does not appear to be restored.

Non-Dystrophin Nuclear Reprogramming In Vivo after Heterologous Fusion

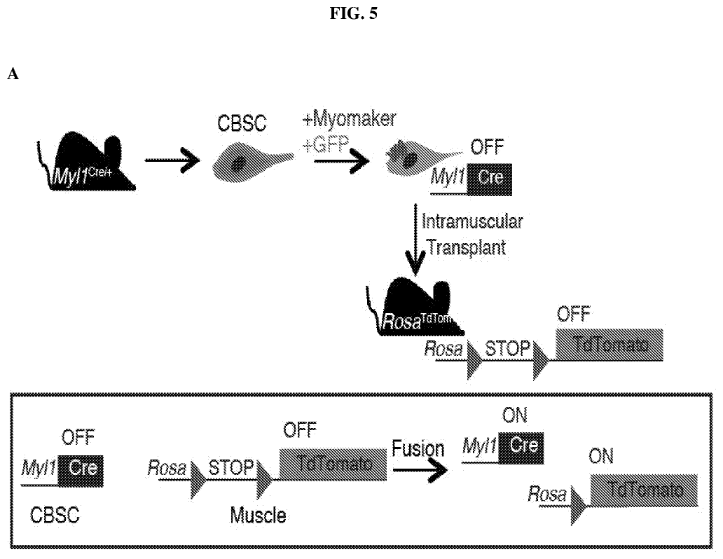

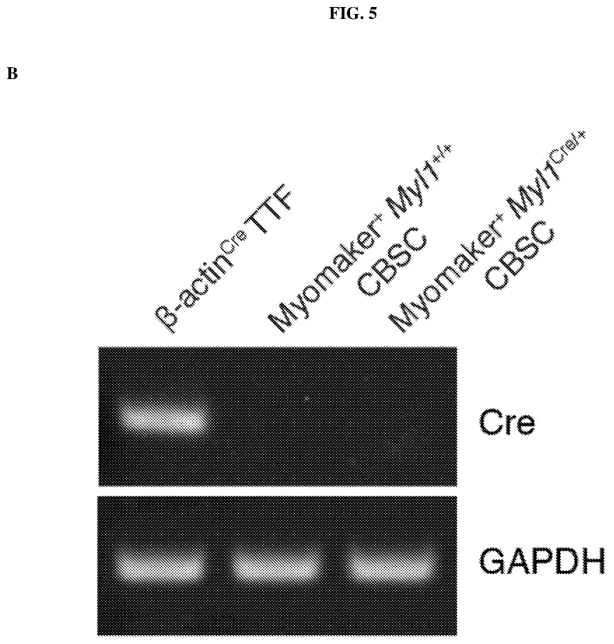

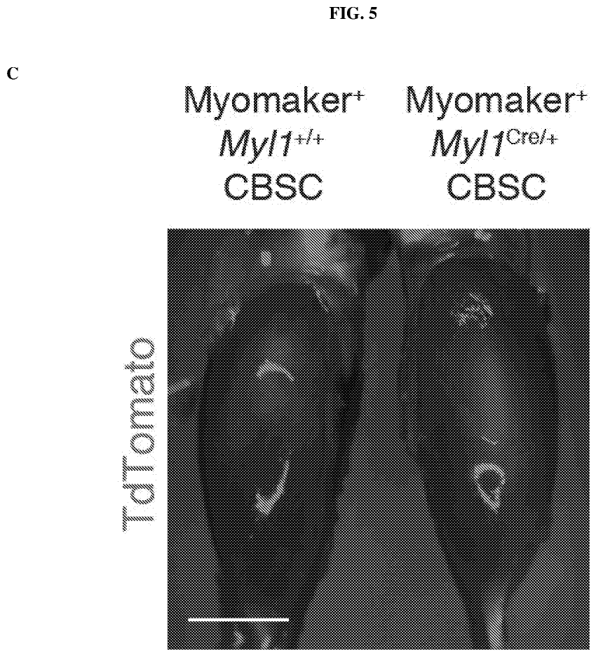

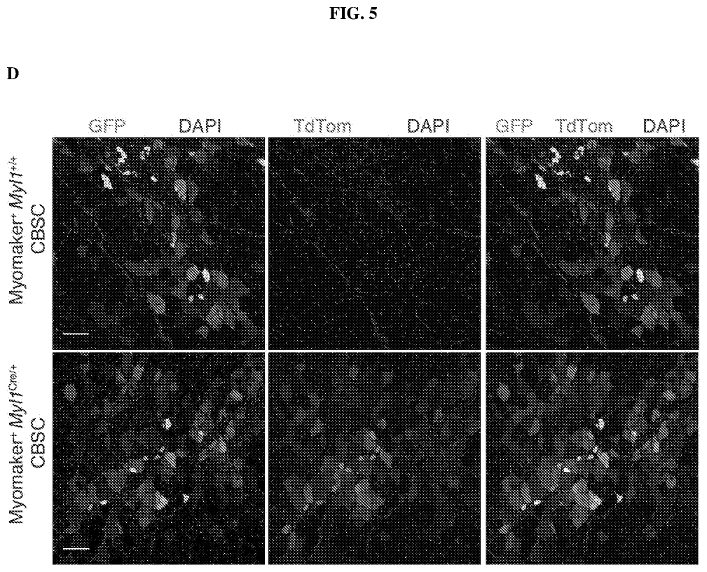

Given our data that heterologous fusion does not induce functional dystrophin reprogramming, we asked whether reprogramming occurs using a dystrophin-independent system. We isolated CBSCs from Myl1^{Cre/+} mice, which express Cre under control of the skeletal muscle-specific myosin, light polypeptide 1 (Myl1 or MLC1f) locus. Myl1^{Cre/+} CBSCs were infected with myomaker and GFP retroviruses and then transplanted into CTX-injured TAs of Rosa26^{tdTomato} mice, which harbor a Cre-dependent tdTomato cassette (FIG. 5A). Thus, for reprogramming in this system the factors necessary for Myl1 expression would have to diffuse into the CBSC nuclei since these nuclei do not normally express Myl1. This would activate Cre in CBSC nuclei and then Cre would need to migrate into the endogenous Rosa26^{tdTomato} myonuclei to induce tdTomato expression (FIG. 5A). We first made certain that GFP⁺ myomaker⁺ Myl1^{Cre/+} CBSCs did not express Cre prior to transplantation. Indeed, RT-PCR for Cre was not detected in Myl1^{Cre/+} CBSCs but was clearly detected in cells that express a ubiquitous β-actin-Cre construct, which served as a positive control for Cre PCR (FIG. 5B). Whole mount fluorescence imaging of TA muscles of Rosa26^{tdTomato} mice revealed that transplantation of myomaker⁺ Myl1^{Cre/+} CBSCs, but not myomaker⁺ Myl1^{+/+} CBSCs, induced tdTomato expression (FIG. 5C). Cryosectioning also demonstrated that while myomaker⁺ Myl1^{+/+} CBSCs fused to muscle (GFP⁺ myofibers), tdTomato expression was not observed (FIG. 5D). After fusion of myomaker⁺ Myl1^{Cre/+} CBSCs co-localization of GFP and tdTomato was detected indicating efficient reprogramming (FIG. 5D). Overall, these findings suggest that muscle-dependent reprogramming occurs in vivo but is likely dependent on the locus of a particular gene.

Discussion

In some of the data, we examine fusion of various non-muscle cells with muscle and subsequent nuclear reprogramming. One of several goals of this work was to evaluate the efficiency of heterologous fusion to restore dystrophin expression in mdx^{4cv} mice as proof-of-concept for using this strategy as a gene delivery vehicle. We used the muscle-specific fusion factor, myomaker, to enhance fusion of MSCs, CBSCs, and TTFs with skeletal muscle. While each of the cell types were able to fuse to muscle after myomaker expression, dystrophin restoration was detected in a subset of cultured myotubes but not in myofibers of mdx^{4cv} mice. However, adult myofibers are able to undergo reprogramming as myomaker CBSCs that express Cre from the muscle-specific Myl1 locus were able to activate Cre-dependent tdTomato expression in myofiber nuclei after fusion.

We demonstrate that MSCs and CBSCs can be suitable vehicles for myomaker-based gene delivery. MSCs exhibit some clinically relevant characteristics for their use in cell therapy since they are readily available from bone marrow or adipose tissue, and can be expanded and propagated in the absence of genomic instabilities. Moreover, MSCs could be used in allogeneic settings since they express minimal MHC class I and II, and they exhibit immunomodulatory properties that would be an added benefit to their use in myomaker-based heterologous fusion. CBSCs show beneficial effects on cardiac injury by secretion of trophic factors after engraftment in mice. In the current study, 5% of myofibers in an uninjured mdx^{4cv} muscle fused with myomaker-expressing non-muscle cells after a single transplantation.

Reprogramming of a differentiated cell can be accomplished through expression of transcription factors or by cell fusion. Indeed, expression of MyoD in fibroblasts is sufficient for conversion to muscle and ectopic expression of defined factors into somatic cells induces transformation to pluripotency. In cell culture, heterokaryon formation between muscle cells and fibroblasts or hepatocytes results in activation of muscle genes from the non-muscle nuclei. Mouse retinal neurons undergo reprogramming after fusion with hematopoietic progenitors during injury, which ultimately results in partial regeneration of the retina.

In conclusion, myomaker allows multiple cell types to fuse to WT and dystrophic muscle in vivo. Myomaker-mediated fusion of non-muscle cells may assist in the delivery of therapeutic material to dystrophic myofibers through the use of reprogramming independent strategies.

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner. Indeed, various modifications of the invention in addition to those described herein will become apparent to those skilled in the art from the foregoing description and the accompanying figures. Such modifications are intended to fall within the scope of the appended claims.

What is claimed is:

1. A method for administering a modified cell to an animal comprising
administering a modified cell to an animal;
wherein
the modified cell expresses a myomaker polypeptide and expresses a dystrophin polypeptide.

2. The method of claim 1, wherein the modified cell overexpresses the dystrophin polypeptide.

3. (canceled)

4. The method of claim 1, wherein the dystrophin polypeptide is a microdystrophin or a minidystrophin.

5. The method of claim 1, wherein the modified cell is a modified animal cell, a modified vertebrate cell, a modified mammalian cell, a modified human cell, a modified rat cell, a modified mouse cell, a modified muscle cell, a modified non-muscle cell, a modified myoblast, a modified fibroblast, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell.

6. The method of claim 1, the modified cell is a modified myoblast, a modified fibroblast, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell.

7. The method of claim 1, wherein the modified cell is an MSC cell which expresses a myomaker polypeptide and overexpresses a dystrophin polypeptide.

8. The method of claim 1, wherein the administering is parenteral administration, mucosal administration, intravenous administration, depot injection, subcutaneous administration, topical administration, intradermal administration, oral administration, sublingual administration, intranasal administration, or intramuscular administration.

9. The method of claim 1, wherein the administering is an injection or an intramuscular injection.

10. The method of claim 1, wherein the animal is selected from mammals, primates, monkeys, macaque, rhesus macaque, or pig tail macaque, humans, canine, feline, bovine, porcine, avian, chicken, mice, rabbits, and rats.

11. The method of claim 1, wherein the animal is a mouse, rat, or human.

12. The method of claim 1, wherein the animal is in need of treatment of a disease.

13. The method of claim 1, wherein the disease is a disease where the animal's cells underexpress dystrophin, do not express dystrophin, or express a defective form of dystrophin.

14. The method of claim 1, wherein the disease is myopathy, muscular dystrophy, amyotrophic lateral sclerosis (ALS or also called Lou Gehrig's disease), glycogen storage disease type II (also called Pompe disease), rhabdomyosarcoma (RMS), or sarcopenia.

15. The method of claim 1, wherein the disease is muscular dystrophy.

16. A modified cell which expresses a myomaker polypeptide and expresses a dystrophin polypeptide.

17. The modified cell of claim 16, wherein the modified cell the dystrophin polypeptide.

18. (canceled)

19. The modified cell of claim 16, wherein the dystrophin polypeptide is a microdystrophin or a minidystrophin.

20. The modified cell of claim 16, wherein the modified cell is a modified animal cell, a modified vertebrate cell, a modified mammalian cell, a modified human cell, a modified rat cell, a modified mouse cell, a modified muscle cell, a modified non-muscle cell, a modified myoblast, a modified fibroblast, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell.

21. The modified cell of claim 16, the modified cell is a modified myoblast, a modified fibroblast, a C2C12 cell, a modified C2C12 cell, a 10T ½ fibroblast, a modified 10T ½ fibroblast, a modified NIH/3T3 cell, a modified CHO cell, a modified mesenchymal stem cell (MSC), a modified hematopoietic stem cell, a modified blood cell, a modified bone marrow cell, a modified stem cell, or a modified adipose stem cell.

22. The modified cell of claim 16, wherein the modified cell is an MSC cell which expresses a myomaker polypeptide and overexpresses a dystrophin polypeptide.
